(19) United States
(12) Patent Application Publication
(10) Pub. No.: US 2026/0227413 A1
(43) Pub. Date: Aug. 6, 2026

(54) USES OF IL-40 AND METHODS FOR DETECTING IL-40 ACTIVITY

(71) Applicant: BioLegend, Inc., San Diego, CA (US)

(72) Inventors: Marcela Hernandez Ruiz, San Diego, CA (US); Gerardo Arrevillaga Boni, San Diego, CA (US); David Michael Soper, San Diego, CA (US)

(21) Appl. No.: 19/529,845

(22) Filed: Feb. 4, 2026

Related U.S. Application Data
(62) Division of application No. 17/393,340, filed on Aug. 3, 2021, now Pat. No. 12,590,973.
Application No. 17/393,340 is a continuation of application No. PCT/US2020/016778, filed on Feb. 5, 2020.
(60) Provisional application No. 62/803,037, filed on Feb. 8, 2019.

(51) Int. Cl. G01N 33/68

(52) U.S. Cl. CPC G01N 33/6869; G01N 2333/5428

(57) ABSTRACT

The technology relates in part to methods for detecting the activity of IL-40 and modified versions thereof. The technology also relates in part to uses of IL-40 for promoting cell differentiation.

### RELATED PATENT APPLICATION(S)

This application claims benefit and is a Divisional of application Ser. No. 17/393,340 filed Aug. 3, 2021, which is a continuation application of PCT/US2020/016778, filed Feb. 5, 2020, which claims benefit of U.S. Provisional Patent Application No. 62/803,037 filed on Feb. 8, 2019, which applications are incorporated herein by reference in their entirety.

### REFERENCE TO AN ELECTRONIC SEQUENCE LISTING

The contents of the electronic sequence listing (102738-1541083-000720US-SL.xml; Size: 81,231 bytes; and Date of Creation: Feb. 17, 2026) is herein incorporated by reference in its entirety.

### FIELD

The technology relates in part to methods for detecting the activity of IL-40 and modified versions thereof. The technology also relates in part to uses of IL-40 for promoting cell differentiation.

### BACKGROUND

Cytokines are small secreted proteins involved in immune responses, host defense, inflammation, and immune system development. Cytokines generally exert their effects by binding specific receptors on the membrane of target cells. Examples of cytokines include interleukins, chemokines, interferons, and members of the tumor necrosis factor superfamily. Certain cytokines are involved in autoimmune diseases, cancer, endocrine disorders, and other ailments; and may be useful for immunotherapy.

IL-40 is generally considered a B cell-associated cytokine, and may be useful for certain research applications (e.g., studying the pathogenesis of certain diseases; studying immune cell activation and differentiation), diagnostics, and/or certain types of immunotherapy. Provided herein are methods for detecting the activity of IL-40 (e.g., recombinant IL-40, and modified versions thereof). IL-40 may be involved in promoting differentiation of certain immune cells. Provided herein are methods for inducing immune cell differentiation using IL-40.

### SUMMARY

Provided herein, in some aspects, are methods for assessing activity of an IL-40 polypeptide comprising a) contacting a cell with a first composition comprising an IL-40 polypeptide; b) measuring production by the cell of one or more cytokines, chemokines, and/or growth factors chosen from CCL2, CCL3, CCL4, CCL5, CCL11, CXCL8, CXCL10, IL-1RA, erythropoietin, PDGF-AA, and VEGF, thereby measuring cytokine, chemokine, and/or growth factor production; and c) detecting the activity of the IL-40 polypeptide in the first composition according to the cytokine, chemokine, and/or growth factor production measured in (b).

Also provided herein, in some aspects, are methods for assessing activity of an IL-40 polypeptide comprising a) contacting a cell with a first composition comprising an IL-40 polypeptide and a second composition comprising a co-stimulant; b) measuring production by the cell of one or more cytokines, chemokines, and/or growth factors chosen from CCL2, CCL3, CCL4, CCL5, CCL11, CCL17, CCL20, CXCL1, CXCL5, CXCL8, CXCL9, CXCL10, CXCL11, IL-1RA, IL-6, erythropoietin, PDGF-AA, and VEGF, thereby measuring cytokine, chemokine, and/or growth factor production; and c) detecting the activity of the IL-40 polypeptide in the first composition according to the cytokine, chemokine, and/or growth factor production measured in (b).

Also provided herein, in some aspects, are methods for assessing activity of an IL-40 polypeptide comprising a) contacting a population of monocytes with a first composition comprising an IL-40 polypeptide; b) detecting monocyte to macrophage differentiation and/or monocyte activation in the population; and c) assessing the activity of the IL-40 polypeptide in the first composition according to the monocyte to macrophage differentiation and/or monocyte activation detected in (b).

Also provided herein, in some aspects, are methods for inducing differentiation of a monocyte to a macrophage, comprising contacting a monocyte with a first composition comprising an IL-40 polypeptide.

Also provided herein, in some aspects, are kits, comprising a) a first composition comprising one or more polypeptides chosen from IFN-γ, GM-CSF, IFN-α, IL-1β, IL-4, IL-10, IL-13, M-CSF, TGF-β, and TNF-α; b) one or more components for measuring cytokine, chemokine, and/or growth factor production, where the cytokines, chemokines and/or growth factors are chosen from one or more of CCL2, CCL3, CCL4, CCL5, CCL11, CCL17, CCL20, CXCL1, CXCL5, CXCL8, CXCL9, CXCL10, CXCL11, IL-1RA, IL-6, erythropoietin, PDGF-AA, and VEGF; and c) instructions for use.

Also provided herein, in some aspects, are methods for detecting the presence of IL-40 receptor on a cell comprising a) contacting the cell with a first composition comprising a stimulant and a second composition comprising recombinant human IL-40 polypeptide; b) incubating the cell with a labeled anti-IL-40 antibody; and c) measuring the amount of labeled anti-IL-40 antibody that binds the cell, thereby detecting the presence of the IL-40 receptor.

Certain embodiments are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows an effect of rhIL-40 on the production of CXCL8 and its activity exacerbation by IFN-γ. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 2 shows an effect of rhIL-40 plus IFN-γ on the production of CXCL9. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 3 shows an effect of rhIL-40 on the production of CCL2 and its activity exacerbation by IFN-γ. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 4 shows an effect of rhIL-40 on the production of CCL3 and its activity exacerbation by IFN-γ. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 5 shows an effect of rhIL-40 on the production of CCL4 and its activity exacerbation by IFN-γ. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 6 shows an effect of rhIL-40 on the production of CCL5 and its activity exacerbation by IFN-γ. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 7 shows an effect of rhIL-40 plus IFN-γ on the production of CXCL10. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 8 shows an effect of rhIL-40 plus IFN-γ on the production of IL1RA. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 9 shows an effect of rhIL-40 plus IFN-γ on the production of IL-6. Results are shown as the media±SEM, and stimuli are represented as follows: Fc fraction (dashed bars), Fc fraction plus IFN-γ (black bars), rhIL-40 (white bars), and rhIL-40 plus IFN-γ (dotted bars).

FIG. 10 shows an effect of rhIL40 on the production of erythropoietin and its activity exacerbation by TGF-β, M-CSF, IL-10, GM-CSF, IFN-γ, or IL-1β. Results are shown as the media±SEM, and stimuli are represented as follows: control or cytokine (white bars), Fc fraction or Fc fraction+cytokine (black bars), rhIL40 or rhIL40+cytokine (dotted bars).

FIG. 11 shows an effect of rhIL40 on the production of PDGF-AA and its activity exacerbation by TGF-β, IL-10, or IL-1β. Results are shown as the media±SEM, and stimuli are represented as follows: control or cytokine (white bars), Fc fraction or Fc fraction+cytokine (black bars), rhIL40 or rhIL40+cytokine (dotted bars).

FIG. 12 shows an effect of rhIL40 on the production of VEGF and its activity exacerbation by IL-4, IL-13, M-CSF, GM-CSF, TNF-α, or IL-1β. Results are shown as the media 1 SEM, and stimuli are represented as follows: control or cytokine (white bars), Fc fraction or Fc fraction+cytokine (black bars), rhIL40 or rhIL40+cytokine (dotted bars).

FIG. 13, Panels A-M, shows an effect of rhIL-40 on the production of chemokines in PBMCs. Statistically significant differences in the production of CXCL8 (Panel A), CXCL10 (Panel B), CCL11 (Panel C), CCL17 (Panel D), CCL2 (Panel E), CCL3 (Panel G), CXCL5 (Panel I), CCL20 (Panel J), CXCL1 (Panel K), CXCL11 (Panel L), and CCL4 (Panel M) were observed in PBMCs. Results are shown as the media±SEM.

FIG. 14, panels A-F, shows THP-1 morphological changes induced by rhIL-40, in the presence or absence of IFN-γ.

FIG. 15, panels A-C, shows an effect of rhIL-40 plus IFN-γ on the expression of HLA-A, B, C (Panel A), CD40 (Panel B), and CD11b (Panel C) on the surface of THP-1 monocytes. Results are shown as histograms, and stimuli are represented as follows: unstimulated cells (dotted gray line), Fc fraction (dashed gray line), rhIL-40 (solid gray line), IFN-γ (dotted black line), Fc fraction plus IFN-γ (dashed black line), and rhIL-40 plus IFN-γ (solid black line).

FIG. 16 shows an effect of rhIL40 on the expression of IL40 receptor on THP-1 cells. Results are shown as histograms, and represented as follows: unstained cells (dotted gray line), rhFc fraction+isotype−APC (dashed gray line), rhFc fraction+anti Fc−APC (solid gray line), rhIL40+isotype−APC (dashed black line), rhIL40+anti Fc−APC (solid black line).

### DETAILED DESCRIPTION

IL-40 may useful for certain research applications, diagnostics, and/or immunotherapy. Accordingly, bioassays for assessing the activity of IL-40, recombinant IL-40, IL-40 variants, IL-40 fragments, and other modified versions of IL-40 would be useful for developing practical applications for IL-40. Provided herein are methods for assessing the activity of IL-40. Also provided herein are methods for inducing cell differentiation using IL-40.

### IL-40

Provided herein are methods for assessing the activity of an interleukin-40 (IL-40) polypeptide. Also provided herein are methods for inducing cell differentiation using interleukin-40 (IL-40). Interleukin-40, which may be referred to as IL-40, IL40, C17orf99 (chromosome 17 open reading frame 99), GLPG464, or UNQ464, is a cytokine involved in the regulation of humoral immunity and is generally associated with B cells. IL-40 is expressed in bone marrow, fetal liver, and certain human B cell lymphomas, and IL-40 expression may be induced in peripheral B cells upon activation. IL-40 is present in mammalian genomes, and the IL-40 gene (C17orf99) encodes a small (~27-kDa) secreted protein unrelated to other cytokine families, indicating a function in mammalian immune responses. Naive B cells can express IL-40 upon activation, and production increases following culture with various cytokines, such as IL-4 and TGF-β1.

IL-40 generally is present in mammals, including human, mouse, rat, and chimpanzee. An example human IL-40 nucleic acid sequence is provided herein as SEQ ID NO: 2 (GENBANK Accession No. NM_001163075.1), and an example human IL-40 amino acid sequence is provided herein as SEQ ID NO: 1 (GENBANK Accession No. NP_001156547.1). An example mouse IL-40 nucleic acid sequence is provided herein as SEQ ID NO: 4 (GENBANK Accession No. NM_029964.1), and an example mouse IL-40 amino acid sequence is provided herein as SEQ ID NO: 3 (GENBANK Accession No. NP_084240.1).

An IL-40 polypeptide may refer to a precursor IL-40 polypeptide (includes the signal peptide) or a mature IL-40 polypeptide (excludes the signal peptide). In some embodiments, an IL-40 polypeptide is a precursor IL-40 polypeptide (e.g., a precursor human IL-40 polypeptide comprising amino acids 1-265 of SEQ ID NO: 1; a precursor mouse IL-40 polypeptide comprising amino acids 1-252 of SEQ ID NO: 3). In some embodiments, an IL-40 polypeptide is a mature IL-40 polypeptide (e.g., a mature human IL-40 polypeptide comprising amino acids 21-265 of SEQ ID NO: 1; a mature mouse IL-40 polypeptide comprising amino acids 19-252 of SEQ ID NO: 3).

In some embodiments, an IL-40 polypeptide is a recombinant IL-40 polypeptide. A recombinant IL-40 polypeptide typically is an IL-40 polypeptide encoded by DNA (i.e., IL-40 nucleic acid sequence) that has been cloned in a vector or system that supports expression of the DNA and translation of messenger RNA. In some embodiments, an IL-40 polypeptide is a recombinant human IL-40 polypeptide (rhIL-40). In some embodiments, an IL-40 polypeptide is a recombinant mouse IL-40 polypeptide (rmIL-40).

An IL-40 polypeptide herein may refer to an unmodified IL-40 polypeptide. An unmodified polypeptide generally refers to a native or wild-type full-length (precursor or mature) polypeptide having no amino acid substitutions, no insertions, no deletions, no chemical modifications, no amino acid side-chain modifications, no tags, no detectable labels, no fusions, and the like.

An IL-40 polypeptide herein may refer to a modified IL-40 polypeptide. A modified polypeptide generally refers to a polypeptide comprising one or more amino acid substitutions, one or more insertions, one or more deletions, one or more chemical modifications, one or more amino acid side-chain modifications, one or more tags, one or more detectable labels, one or more fusions, and the like and combinations thereof. Modifications may include, for example, addition of one or more fluorophores, glycosylation, prenylation, PEGylation, attachment to a solid surface, biotinylation, antibody conjugation, conjugation to a therapeutic agent, chemical modifications at cysteine (e.g., aminoethylation, iodoacetamides, maleimides, Dha formation, disulfide formation, reaction of Dha with thiols, and desulfurization of disulfides), incorporation of one or more unnatural amino acids, and the like and combinations thereof.

In some embodiments, an IL-40 polypeptide refers to an IL-40 variant or mutant. Such variants include, for example, deletions from, and/or insertions into and/or substitutions of, residues within the amino acid sequences of an IL-40 polypeptide. An IL-40 variant may include any combination of deletion, insertion, and substitution. In some embodiments, an IL-40 polypeptide comprises one or more amino acid substitutions. These variants have at least one amino acid residue removed from the IL-40 polypeptide and a different residue inserted in its place. For example, an IL-40 variant may comprise 1, 2, 3, 4, 5, 6, 7, 8, 9, 10 or more amino acid substitutions. An IL-40 variant may include conservative substitutions and/or non-conservative substitutions, and the variants may be screened using one or more bioassays described herein for assessing IL-40 activity. Examples of substitutions are listed below:

### Example Amino Acid Residue Substitutions

Ala (A) val; leu; ile; val
Arg (R) lys; gln; asn; lys
Asn (N) gln; his; asp, lys; gln; arg
Asp (D) glu; asn
Cys (C) ser; ala
Gln (Q) asn; glu
Glu (E) asp; gln
Gly (G) ala
His (H) asn; gln; lys; arg
Ile (I) leu; val; met; ala; leu; phe; norleucine
Leu (L) norleucine; ile; val; ile; met; ala; phe
Lys (K) arg; gln; asn
Met (M) leu; phe; ile
Phe (F) leu; val; ile; ala; tyr
Pro (P) ala
Ser (S) thr
Thr (T) ser
Trp (W) tyr; phe
Tyr (Y) trp; phe; thr; ser
Val (V) ile; leu; met; phe; ala; norleucine

Substantial modifications in the biological properties of an IL-40 polypeptide may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, and/or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions typically entail exchanging a member of one of these classes for another class.

In some embodiments, an IL-40 polypeptide comprises one or more insertions. In some embodiments, an IL-40 polypeptide comprises one or more insertions, where each insertion comprises one or more amino acids. For example, each insertion may comprise 1, 2, 3, 4, 5, 6, 7, 8, 9, 10 or more inserted amino acids. In some embodiments, an IL-40 polypeptide comprises one or more deletions. In some embodiments, an IL-40 polypeptide comprises one or more deletions, where each deletion removes one or more amino acids from the full length amino acid sequence. For example, each deletion may remove 1, 2, 3, 4, 5, 6, 7, 8, 9, 10 or more amino acids.

In some embodiments, an IL-40 polypeptide comprises a fused polypeptide. A fused polypeptide may be referred to as a fusion protein or chimeric protein. Fused polypeptides typically are created through the joining of two or more genes that code for separate proteins. Translation of this fusion construct may result in a single or multiple polypeptides with functional properties derived from each of the original proteins. Recombinant fusion proteins may be created artificially by recombinant DNA technology for use in biological research or therapeutics. Examples of fused polypeptides include IL-40 fused with a fluorescent protein tag (e.g., green fluorescent protein (GFP)), therapeutic protein (e.g., antibody), or any protein tag described herein. In some embodiments a fused polypeptide comprises a linker (e.g., flexible linker, rigid linker, cleavable linker).

In some embodiments, an IL-40 polypeptide comprises one or more tags (e.g., one or more amino acid or peptide tags; one or more affinity tags). Tags may facilitate detection, isolation and/or purification of an IL-40 polypeptide. A tag sometimes specifically binds a molecule or moiety of a solid phase or a detectable label, for example, thereby having utility for isolating, purifying and/or detecting an IL-40 polypeptide. In some embodiments, a tag comprises one or more of the following elements: Fc (derived from immunoglobulin Fc domain), FLAG (e.g., DYKDDDDKG (SEQ ID NO: 48)), V5 (e.g., GKPIPNPLLGLDST (SEQ ID NO: 49)), c-MYC (e.g., EQKLISEEDL (SEQ ID NO: 50)), HSV (e.g., QPELAPEDPED (SEQ ID NO: 51)), influenza hemagglutinin, HA (e.g., YPYDVPDYA (SEQ ID NO: 52)), VSV-G (e.g., YTDIEMNRLGK (SEQ ID NO: 53)), bacterial glutathione-S-transferase, maltose binding protein, a streptavidin- or avidin-binding tag (e.g., pcDNA™ 6 BioEase™ Gateway® Biotinylation System (Invitrogen)), thioredoxin, β-galactosidase, VSV-glycoprotein, a fluorescent protein (e.g., green fluorescent protein or one of its many color variants (e.g., yellow, red, blue)), a polylysine or polyarginine sequence, a polyhistidine sequence (e.g., His6 (SEQ ID NO: 54)) or other sequence that chelates a metal (e.g., cobalt, zinc, copper), and/or a cysteine-rich sequence that binds to an arsenic-containing molecule. In certain embodiments, a cysteine-rich tag comprises the amino acid sequence CC-Xn-CC (SEQ ID NO: 55), where X is any amino acid and n is 1 to 3, and the cysteine-rich sequence sometimes is CCPGCC (SEQ ID NO: 56). In certain embodiments, the tag comprises a cysteine-rich element and a polyhistidine element (e.g., CCPGCC (SEQ ID NO: 56) and His6 (SEQ ID NO: 54)).

A tag may bind to a binding partner. For example, some tags bind to an antibody (e.g., FLAG) and sometimes specifically bind to a small molecule. For example, a polyhistidine tag specifically chelates a bivalent metal, such as copper, zinc and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ (EDT2[4′,5′-bis(1,3,2-dithioarsolan-2-yl)fluorescein-(1,2-ethanedithiol)2]) and ReAsH reagents. Such antibodies and small molecules sometimes are linked to a solid phase for isolation of the target protein or target peptide.

In some embodiments, an IL-40 polypeptide comprises one or more detectable markers or labels. In some embodiments, an IL-40 polypeptide is conjugated to a detectable marker or label. For example, for research and diagnostic applications, a modified IL-40 polypeptide may be labeled with a detectable moiety. Numerous labels are available which generally include radioisotopes (e.g., ³⁵S, ¹⁴C, ¹²⁵I, ³H, and ¹³¹I), fluorescent labels (e.g., rare earth chelates (europium chelates) or fluorescein and its derivatives, rhodamine and its derivatives, dansyl, Lissamine, phycoerythrin, Texas Red and Brilliant Violet™), and enzyme-substrate labels (e.g., described in U.S. Pat. No. 4,275,149, which is incorporated by reference herein, luciferases (e.g., firefly luciferase and bacterial luciferase; U.S. Pat. No. 4,737,456, which is incorporated by reference herein), luciferin, 2,3-dihydrophthalazinediones, malate dehydrogenase, urease, peroxidase such as horseradish peroxidase (HRP), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like).

In certain instances, a label is indirectly conjugated to an IL-40 polypeptide. For example, an IL-40 polypeptide may be conjugated with biotin and any suitable label mentioned above may be conjugated with avidin, or vice versa. Biotin binds selectively to avidin and thus, the label can be conjugated with an IL-40 polypeptide in this indirect manner. Alternatively, to achieve indirect conjugation of a label with an IL-40 polypeptide, the IL-40 polypeptide is conjugated with a small hapten (e.g., digoxin) and one of the types of labels mentioned above is conjugated with an anti-hapten antibody (e.g., anti-digoxin antibody).

In some embodiments, an IL-40 polypeptide refers to a fragment of an IL-40 polypeptide. Generally, an IL-40 fragment contains fewer amino acids than a full-length mature IL-40. For example, an IL-40 fragment may include a portion of the mature human IL-40 polypeptide (i.e., a portion of amino acids 21-265 of SEQ ID NO: 1), or a portion of the mature mouse IL-40 polypeptide (i.e. a portion of amino acids 19-252 of SEQ ID NO: 3). Full-length mature human IL-40 is 245 amino acids in length. Accordingly, fragments of human IL-40 may be 244 amino acids in length or shorter. Full-length mature mouse IL-40 is 234 amino acids in length. Accordingly, fragments of mouse IL-40 may be 233 amino acids in length or shorter.

In some embodiments, an IL-40 polypeptide refers to a functional fragment of an IL-40 polypeptide. Methods for assessing the activity of IL-40 polypeptides and functional fragments of IL-40 are provided herein. In some embodiments, a functional fragment of IL-40 is a fragment that exhibits at least 50% of the activity of a full-length mature IL-40. For example, a functional fragment of IL-40 is a fragment that exhibits at least 50%, at least 60%, at least 70%, at least 80%, at least 90%, at least 95%, or more of the activity of a full-length mature IL-40.

In some embodiments, an IL-40 polypeptide is immobilized on a solid support or substrate. In some embodiments, an IL-40 polypeptide is non-diffusively immobilized on a solid support (e.g., the IL-40 polypeptide does not detach from the solid support). A solid support or substrate can be any physically separable solid to which an IL-40 polypeptide can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™ polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support or substrate can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). An IL-40 polypeptide may be linked to a solid support by covalent bonds or by non-covalent interactions and may be linked to a solid support directly or indirectly (e.g., via an intermediary agent such as a spacer molecule or biotin).

### Stimulants and Co-Stimulants

Certain methods provided herein include use of a stimulant or co-stimulant. Use of stimulants and co-stimulants may be included, for example, in methods for assessing the activity of an IL-40 polypeptide, and/or in methods for inducing cell differentiation (e.g., inducing differentiation of a monocyte to a macrophage). A stimulant may be used in certain instances (e.g., to stimulate a cell prior to exposure to IL-40). A co-stimulant may be used in certain instances (e.g., to co-stimulate a cell during IL-40 exposure). In some embodiments, a cell or population of cells is contacted with a stimulant/co-stimulant. In some embodiments, a cell or population of cells is simultaneously contacted with a stimulant/co-stimulant and an IL-40 polypeptide. In some embodiments, a cell or population of cells is contacted with a stimulant/co-stimulant prior to being contacted with an IL-40 polypeptide.

In certain instances, a stimulant/co-stimulant can strengthen or enhance the effect of IL-40 on a cell or a population of cells. For example, a stimulant/co-stimulant can enhance production of certain cytokines, chemokines, and/or growth factors in response to IL-40 stimulation. A stimulant/co-stimulant also can enhance certain types of cell differentiation in response to IL-40 stimulation. In certain instances, a stimulant/co-stimulant can provide a synergistic enhancement when combined with IL-40. For example, a stimulant/co-stimulant can synergistically enhance production of certain cytokines, chemokines, and/or growth factors in response to IL-40 stimulation. A stimulant/co-stimulant also can synergistically enhance certain types of cell differentiation in response to IL-40 stimulation. An enhancement afforded by a stimulant or co-stimulant may be additive, multiplicative, or exponential.

Any suitable stimulant/co-stimulant may be used in conjunction with the methods provided herein. In some embodiments, a stimulant/co-stimulant comprises a soluble/secreted protein. In some embodiments, a stimulant/co-stimulant comprises a cytokine. In some embodiments, a stimulant/co-stimulant comprises a chemokine. Non-limiting examples of stimulants/co-stimulants include interferon-γ (IFN-γ), interferon-α (IFN-α), lipopolysaccharides (LPS), granulocyte-macrophage colony-stimulating factor (GM-CSF), interleukin 1 beta (IL-1β), interleukin 4 (IL-4), interleukin 13 (IL-13), macrophage colony-stimulating factor (M-CSF), infection (e.g., fungal infection, helminth infection), immune complexes, interleukin-1 receptor (IL-1R), interleukin 10 (IL-10), transforming growth factor beta (TGF-β), glucocorticoids, interleukin 6 (IL-6), leukemia inhibitory factor (LIF), tumor necrosis factor alpha (TNF-α), adenosine, complement components, and interleukin 32 (IL-32).

### IFN-γ

Certain methods provided herein include use of interferon-γ (IFN-γ) as a stimulant or co-stimulant. Interferon-γ (also referred to as IFN-γ, IFNγ, IFN-g, IFNg, IFN-gamma, interferon gamma, immune interferon, type II interferon) is a dimerized soluble cytokine and a type II class of interferon. IFN-γ generally is involved in innate and adaptive immunity against certain viral, bacterial, and protozoal infections. IFN-γ can function as an activator of macrophages and inducer of Class II major histocompatibility complex (MHC) molecule expression. IFN-γ can inhibit viral replication directly, and can provide immunostimulatory and immunomodulatory effects. Aberrant IFN-γ expression often is associated with a number of autoinflammatory and autoimmune diseases. IFN-γ typically is produced by natural killer (NK) and natural killer T (NKT) cells as part of the innate immune response; by CD4 Th1 and CD8 cytotoxic T lymphocyte (CTL) effector T cells once antigen-specific immunity develops; and by non-cytotoxic innate lymphoid cells (ILC).

Any suitable IFN-γ, or functional fragment, or modified version thereof, may be used in conjunction with the methods described herein (e.g., as a co-stimulant with IL-40; as a stimulant prior to IL-40 exposure). In some embodiments, IFN-γ is a human IFN-γ. An example human IFN-γ nucleic acid sequence is provided herein as SEQ ID NO: 6 (GENBANK Accession No. NM_000619.3), and an example human IFN-γ amino acid sequence is provided herein as SEQ ID NO: 5 (GENBANK Accession No. NP_000610.2). In some embodiments, IFN-γ is a mouse IFN-γ. An example mouse IFN-γ nucleic acid sequence is provided herein as SEQ ID NO: 8 (GENBANK Accession No. NM_008337.4), and an example mouse IFN-γ amino acid sequence is provided herein as SEQ ID NO: 7 (GENBANK Accession No. NP_032363.1).

IFN-γ may refer to a precursor IFN-γ polypeptide (includes the signal peptide) or a mature IFN-γ polypeptide (excludes the signal peptide). In some embodiments, IFN-γ is a precursor IFN-γ polypeptide (e.g., a precursor human IFN-γ polypeptide comprising amino acids 1-166 of SEQ ID NO: 5; a precursor human IFN-γ polypeptide comprising amino acids 1-161 of SEQ ID NO: 5; a precursor mouse IFN-γ polypeptide comprising amino acids 1-155 of SEQ ID NO: 7). In some embodiments, an IFN-γ polypeptide is a mature IFN-γ polypeptide (e.g., a mature human IFN-γ polypeptide comprising amino acids 24-166 of SEQ ID NO: 5; a mature human IFN-γ polypeptide comprising amino acids 24-161 of SEQ ID NO: 5; a mature mouse IFN-γ polypeptide comprising amino acids 23-155 of SEQ ID NO: 7).

In some embodiments, IFN-γ is a recombinant IFN-γ polypeptide. A recombinant IFN-γ polypeptide typically is an IFN-γ polypeptide encoded by DNA (i.e., IFN-γ nucleic acid sequence) that has been cloned in a vector or system that supports expression of the DNA and translation of messenger RNA. In some embodiments, IFN-γ is a recombinant human IFN-γ polypeptide (rhIFN-γ). In some embodiments, IFN-γ is a recombinant mouse IFN-γ polypeptide (rmIFN-γ).). In some embodiments, IFN-γ is a commercially available recombinant human IFN-γ (e.g., BioLegend cat #570202). In some embodiments, IFN-γ is a commercially available recombinant mouse IFN-γ (e.g., BioLegend cat #575302).

IFN-γ may refer to an unmodified IFN-γ polypeptide, a modified IFN-γ polypeptide, an IFN-γ variant, an IFN-γ mutant; or a fragment thereof or a functional fragment thereof. Unmodified polypeptides, modified polypeptides, mutants, variants, and fragments are described herein. In some embodiments, IFN-γ refers to a polypeptide comprising an amino acid sequence that is at least about 75% identical to SEQ ID NO: 5 or SEQ ID NO: 7. For example, IFN-γ may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 5 or SEQ ID NO: 7.

In some embodiments, IFN-γ refers to a fragment of an IFN-γ polypeptide. Generally, an IFN-γ fragment contains fewer amino acids than a full-length mature IFN-γ. For example, an IFN-γ fragment may include a portion of the mature human IFN-γ polypeptide (e.g., a portion of amino acids 24-166 of SEQ ID NO: 5; a portion of amino acids 24-161 of SEQ ID NO: 5), or a portion of the mature mouse IFN-γ polypeptide (i.e. a portion of amino acids 23-155 of SEQ ID NO: 7). One example full-length mature human IFN-γ is 143 amino acids in length. Accordingly, fragments of human IFN-γ may be 142 amino acids in length or shorter. Another example full-length mature human IFN-γ is 138 amino acids in length. Accordingly, fragments of human IFN-γ may be 137 amino acids in length or shorter. Full-length mature mouse IFN-γ is 133 amino acids in length. Accordingly, fragments of mouse IFN-γ may be 132 amino acids in length or shorter.

In some embodiments, IFN-γ refers to a functional fragment of an IFN-γ polypeptide. Any suitable method for assessing the activity of IFN-γ and functional fragments of IFN-γ may be used to determine whether an IFN-γ fragment is a functional fragment. For example, one assay for assessing IFN-γ activity is the induction of an antiviral state in target cells, sometimes referred to as a cytopathic protection effect (CPE) assay. An example CPE assay uses A549 human lung carcinoma cells challenged with encephalomyocarditis virus (EMCV) and the effects are compared to a standard for human IFN-γ (e.g., Gxg01-902-535, BEI Resources). In some embodiments, a functional fragment of IFN-γ is a fragment that exhibits at least 50% of the activity of an IFN-γ standard or a full-length mature IFN-γ. For example, a functional fragment of IFN-γ is a fragment that exhibits at least 50%, at least 60%, at least 70%, at least 80%, at least 90%, at least 95%, or more of the activity of an IFN-γ standard or a full-length mature IFN-γ.

### IL-4

Certain methods provided herein include use of interleukin 4 (IL-4) as a stimulant or co-stimulant. Interleukin 4 (also referred to as IL-4, B cell growth factor 1 (BCGF-1), B-cell stimulatory factor 1 (BSF-1), interleukin-4, lymphocyte stimulatory factor 1, MGC79402) is the primary cytokine implicated in the development of Th2-mediated responses, which is associated with allergy and asthma. The Type I receptor comprises IL-4Rα and the common gamma-chain (γc), which is also shared by the cytokines IL-2, -7, -9, -15 and -21 and is present in hematopoietic cells. IL-4 can use the type II complex, comprising IL-4Rα and IL-13Rα1, which is present in non-hematopoietic cells. This second receptor complex is a functional receptor for IL-13, which shares approximately 25% homology with IL-4. The type I receptor complex can be formed only by IL-4 and is active in Th2 development. In contrast, the type II receptor complex formed by either IL-4 or IL-13 is more active during airway hypersensitivity and mucus secretion and is not found in T cells.

Any suitable IL-4, or functional fragment, or modified version thereof, may be used in conjunction with the methods described herein (e.g., as a co-stimulant with IL-40; as a stimulant prior to IL-40 exposure). In some embodiments, IL-4 is a human IL-4. An example human IL-4 nucleic acid sequence is provided herein as SEQ ID NO: 13 (full mRNA sequence provided as GENBANK Accession No. NM_000589), and an example human IL-4 amino acid sequence is provided herein as SEQ ID NO: 12 (GENBANK Accession No. NP_000580.1). In some embodiments, IL-4 is a mouse IL-4. An example mouse IL-4 nucleic acid sequence is provided herein as SEQ ID NO: 15 (full mRNA sequence provided as GENBANK Accession No. NM_021283), and an example mouse IL-4 amino acid sequence is provided herein as SEQ ID NO: 14 (GENBANK Accession No. NP_067258.1).

IL-4 may refer to a precursor IL-4 polypeptide (includes the signal peptide) or a mature IL-4 polypeptide (excludes the signal peptide). In some embodiments, IL-4 is a precursor IL-4 polypeptide (e.g., a precursor human IL-4 polypeptide comprising amino acids 1-153 of SEQ ID NO: 12; a precursor mouse IL-4 polypeptide comprising amino acids 1-140 of SEQ ID NO: 14). In some embodiments, an IL-4 polypeptide is a mature IL-4 polypeptide (e.g., a mature human IL-4 polypeptide comprising amino acids 25-153 of SEQ ID NO: 12; a mature mouse IL-4 polypeptide comprising amino acids 21-140 of SEQ ID NO: 14; a mature mouse IL-4 polypeptide comprising amino acids 23-140 of SEQ ID NO: 14).

In some embodiments, IL-4 is a recombinant IL-4 polypeptide. A recombinant IL-4 polypeptide typically is an IL-4 polypeptide encoded by DNA (i.e., IL-4 nucleic acid sequence) that has been cloned in a vector or system that supports expression of the DNA and translation of messenger RNA. In some embodiments, IL-4 is a recombinant human IL-4 polypeptide (rhIL-4). In some embodiments, IL-4 is a recombinant mouse IL-4 polypeptide (rmIL-4). In some embodiments, IL-4 is a commercially available recombinant human IL-4 (e.g., BioLegend cat #574002). In some embodiments, IL-4 is a commercially available recombinant mouse IL-4 (e.g., BioLegend cat #574302).

IL-4 may refer to an unmodified IL-4 polypeptide, a modified IL-4 polypeptide, an IL-4 variant, an IL-4 mutant; or a fragment thereof or a functional fragment thereof. Unmodified polypeptides, modified polypeptides, mutants, variants, and fragments are described herein. In some embodiments, IL-4 refers to a polypeptide comprising an amino acid sequence that is at least about 75% identical to SEQ ID NO: 12 or SEQ ID NO: 14. For example, IL-4 may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 12 or SEQ ID NO: 14.

In some embodiments, IL-4 refers to a fragment of an IL-4 polypeptide. Generally, an IL-4 fragment contains fewer amino acids than a full-length mature IL-4. For example, an IL-4 fragment may include a portion of the mature human IL-4 polypeptide (i.e., a portion of amino acids 25-153 of SEQ ID NO: 12), or a portion of the mature mouse IL-4 polypeptide (i.e. a portion of amino acids 21-140 of SEQ ID NO: 14). An example full-length mature human IL-4 is 129 amino acids in length. Accordingly, fragments of human IL-4 may be 128 amino acids in length or shorter. An example full-length mature mouse IL-4 is 120 amino acids in length. Accordingly, fragments of mouse IL-4 may be 119 amino acids in length or shorter. Another example full-length mature mouse IL-4 is 118 amino acids in length. Accordingly, fragments of mouse IL-4 may be 117 amino acids in length or shorter.

In some embodiments, IL-4 refers to a functional fragment of an IL-4 polypeptide. Any suitable method for assessing the activity of IL-4 and functional fragments of IL-4 may be used to determine whether an IL-4 fragment is a functional fragment. For example, one assay for assessing IL-4 activity is a cell proliferation assay. An example cell proliferation assay measures ED₅₀ of IL-4 according to dose-dependent stimulation of TF-1 cell proliferation. Another example cell proliferation assay measures ED₅₀ of IL-4 according to dose-dependent stimulation of CTLL-2 cell proliferation. In some embodiments, a functional fragment of IL-4 is a fragment that exhibits at least 50% of the activity of an IL-4 standard (e.g., WHO International Standard for Human IL-4 (NIBSC code: 88/656)) or a full-length mature IL-4. For example, a functional fragment of IL-4 is a fragment that exhibits at least 50%, at least 60%, at least 70%, at least 80%, at least 90%, at least 95%, or more of the activity of an IL-4 standard or a full-length mature IL-4.

### IL-10

Certain methods provided herein include use of interleukin 10 (IL-10) as a stimulant or co-stimulant. Interleukin 10 (also referred to as IL-10, B-TCGF, CSIF, TGIF) was first described as a cytokine that is produced by T helper 2 (Th2) cell clones. It inhibits interferon (IFN)-γ synthesis in Th1 cells, and therefore it was initially called cytokine synthesis inhibiting factor (CSIF). Macrophages are the main source of IL-10 and its secretion can be stimulated by endotoxin (via Toll-like receptor 4, NF-κB dependent), tumor necrosis factor TNF-α (via TNF receptor p55, NF-κB-dependent), catecholamines, and IL-1. IL-10 controls inflammatory processes by suppressing the expression of proinflammatory cytokines, chemokines, adhesion molecules, as well as antigen-presenting and costimulatory molecules in monocytes/macrophages, neutrophils, and T cells. IL-10 inhibits the production of proinflammatory mediators by monocytes and macrophages such as endotoxin- and IFN-γ-induced release of IL-1α, IL-6, IL-8, G-CSF, GM-CSF, and TNF-α. In addition, it enhances the production of anti-inflammatory mediators such as IL-1RA and soluble TNFα receptors. IL-10 inhibits the capacity of monocytes and macrophages to present antigen to T cells. This is realized by down-regulation of constitutive and IFNγ-induced cell surface levels of MHC class II, of costimulatory molecules such as CD86 and of some adhesion molecules such as CD58.

Any suitable IL-10, or functional fragment, or modified version thereof, may be used in conjunction with the methods described herein (e.g., as a co-stimulant with IL-40; as a stimulant prior to IL-40 exposure). In some embodiments, IL-10 is a human IL-10. An example human IL-10 nucleic acid sequence is provided herein as SEQ ID NO: 17 (full mRNA sequence provided as GENBANK Accession No. NM_000572), and an example human IL-10 amino acid sequence is provided herein as SEQ ID NO: 16 (GENBANK Accession No. NP_000563.1). In some embodiments, IL-10 is a mouse IL-10. An example mouse IL-10 nucleic acid sequence is provided herein as SEQ ID NO: 19 (full mRNA sequence provided as GENBANK Accession No. NM_010548), and an example mouse IL-10 amino acid sequence is provided herein as SEQ ID NO: 18 (GENBANK Accession No. NP_034678.1).

IL-10 may refer to a precursor IL-10 polypeptide (includes the signal peptide) or a mature IL-10 polypeptide (excludes the signal peptide). In some embodiments, IL-10 is a precursor IL-10 polypeptide (e.g., a precursor human IL-10 polypeptide comprising amino acids 1-178 of SEQ ID NO: 16; a precursor mouse IL-10 polypeptide comprising amino acids 1-178 of SEQ ID NO: 18). In some embodiments, an IL-10 polypeptide is a mature IL-10 polypeptide (e.g., a mature human IL-10 polypeptide comprising amino acids 19-178 of SEQ ID NO: 16; a mature mouse IL-10 polypeptide comprising amino acids 19-178 of SEQ ID NO: 18).

In some embodiments, IL-10 is a recombinant IL-10 polypeptide. A recombinant IL-10 polypeptide typically is an IL-10 polypeptide encoded by DNA (i.e., IL-10 nucleic acid sequence) that has been cloned in a vector or system that supports expression of the DNA and translation of messenger RNA. In some embodiments, IL-10 is a recombinant human IL-10 polypeptide (rhIL-10). In some embodiments, IL-10 is a recombinant mouse IL-10 polypeptide (rmIL-10). In some embodiments, IL-10 is a commercially available recombinant human IL-10 (e.g., BioLegend cat #715602; BioLegend cat #571002). In some embodiments, IL-10 is a commercially available recombinant mouse IL-10 (e.g., BioLegend cat #575802).

IL-10 may refer to an unmodified IL-10 polypeptide, a modified IL-10 polypeptide, an IL-10 variant, an IL-10 mutant; or a fragment thereof or a functional fragment thereof. Unmodified polypeptides, modified polypeptides, mutants, variants, and fragments are described herein. In some embodiments, IL-10 refers to a polypeptide comprising an amino acid sequence that is at least about 75% identical to SEQ ID NO: 16 or SEQ ID NO: 18. For example, IL-10 may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 16 or SEQ ID NO: 18.

In some embodiments, IL-10 refers to a fragment of an IL-10 polypeptide. Generally, an IL-10 fragment contains fewer amino acids than a full-length mature IL-10. For example, an IL-10 fragment may include a portion of the mature human IL-10 polypeptide (i.e., a portion of amino acids 19-178 of SEQ ID NO: 16), or a portion of the mature mouse IL-10 polypeptide (i.e. a portion of amino acids 19-178 of SEQ ID NO: 18). An example full-length mature human IL-10 is 160 amino acids in length. Accordingly, fragments of human IL-10 may be 159 amino acids in length or shorter. An example full-length mature mouse IL-10 is 160 amino acids in length. Accordingly, fragments of mouse IL-10 may be 159 amino acids in length or shorter.

In some embodiments, IL-10 refers to a functional fragment of an IL-10 polypeptide. Any suitable method for assessing the activity of IL-10 and functional fragments of IL-10 may be used to determine whether an IL-10 fragment is a functional fragment. For example, one assay for assessing IL-10 activity is an IFN-γ inhibition assay. An example IFN-γ inhibition assay measures the extent to which IL-10 inhibits the induction of INF-γ in PHA activated human PBMC. Another assay for assessing IL-10 activity involves dose dependent stimulation MC/9 cell proliferation. In some embodiments, a functional fragment of IL-10 is a fragment that exhibits at least 50% of the activity of an IL-10 standard or a full-length mature IL-10. For example, a functional fragment of IL-10 is a fragment that exhibits at least 50%, at least 60%, at least 70%, at least 80%, at least 90%, at least 95%, or more of the activity of an IL-10 standard or a full-length mature IL-10.

### TGF-β

Certain methods provided herein include use of transforming growth factor beta (TGF-β) as a stimulant or co-stimulant. Transforming growth factor beta (also referred to as TGF-β, TGF-β1, TGFB, DPD1, transforming growth factor, Transforming Growth Factor Beta 1, TGF-Beta-1) is synthesized in cells as a 390-amino acid polypeptide. Furin cleaves the protein at residue 278, yielding an N-terminal cleavage product which corresponds to the latency-associated peptide (LAP), and the 25-kD C-terminal portion of the precursor constitutes the mature TGF-β1. TGF-β activators can release TGF-β from LAP. These activators include proteases that degrade LAP, thrombospondin-1, reactive oxygen species, and integrins avb6 and avb8. Mouse TGF-β converts naïve T cells into regulatory T (Treg) cells that prevent autoimmunity. Although human TGF-β1 is widely used for inducing FOXP3+ in vitro, it might not be an essential factor for human Treg differentiation. Th17 murine can be induced from naïve CD4+ T cells by the combination of TGF-β1 and IL-6 or IL-21. Nevertheless, the regulation of human Th17 differentiation is distinct. TGF-β1 seems to have dual effects on human Th17 differentiation in a dose-dependent manner. While TGF-β1 is required for the expression of RORγt, in human naive CD4+ T cells from cord blood, TGF-β1 can inhibit the function of RORγt at high doses. By using serum-free medium, it has been clarified that the optimum conditions for human Th17 differentiation are TGF-β1, IL-1β, and IL-2 in combination with IL-6, IL-21 or IL-23.

Any suitable TGF-β, or functional fragment, or modified version thereof, may be used in conjunction with the methods described herein (e.g., as a co-stimulant with IL-40; as a stimulant prior to IL-40 exposure). In some embodiments, TGF-β is a human TGF-β. An example human TGF-β nucleic acid sequence is provided herein as SEQ ID NO: 21 (full mRNA sequence provided as GENBANK Accession No. NM_000660.7), and an example human TGF-β amino acid sequence is provided herein as SEQ ID NO: 20 (GENBANK Accession No. NP_000651.3). Another example human TGF-β nucleic acid sequence is provided as GENBANK Accession No. BC000125.1, and another example human TGF-β amino acid sequence is provided GENBANK Accession No. P01137. In some embodiments, TGF-β is a mouse TGF-β. An example mouse TGF-β nucleic acid sequence is provided herein as SEQ ID NO: 23 (full mRNA sequence provided as GENBANK Accession No. NM_011577.2), and an example mouse TGF-β amino acid sequence is provided herein as SEQ ID NO: 22 (GENBANK Accession No. NP_035707).

TGF-β may refer to a precursor TGF-β polypeptide (includes the signal peptide) or a mature TGF-β polypeptide (excludes the signal peptide; or excludes the signal peptide and the latency-associated peptide). In some embodiments, TGF-β is a precursor TGF-β polypeptide (e.g., a precursor human TGF-β polypeptide comprising amino acids 1-390 of SEQ ID NO: 20; a precursor mouse TGF-β polypeptide comprising amino acids 1-390 of SEQ ID NO: 22). In some embodiments, a TGF-β polypeptide is a mature TGF-β polypeptide (e.g., a mature human TGF-β polypeptide comprising amino acids 30-390 of SEQ ID NO: 20; a mature human TGF-β polypeptide comprising amino acids 279-390 of SEQ ID NO: 20; a mature mouse TGF-β polypeptide comprising amino acids 29-390 of SEQ ID NO: 22; a mature mouse TGF-β polypeptide comprising amino acids 279-390 of SEQ ID NO: 22).

In some embodiments, TGF-β is a recombinant TGF-β polypeptide. A recombinant TGF-β polypeptide typically is a TGF-β polypeptide encoded by DNA (i.e., TGF-β nucleic acid sequence) that has been cloned in a vector or system that supports expression of the DNA and translation of messenger RNA. In some embodiments, TGF-β is a recombinant human TGF-β polypeptide (rhTGF-β). In some embodiments, TGF-β is a recombinant mouse TGF-β polypeptide (rmTGF-β). In some embodiments, TGF-β is a commercially available recombinant human TGF-β (e.g., BioLegend cat #580704; BioLegend cat #781802). In some embodiments, TGF-β is a commercially available recombinant mouse TGF-β (e.g., BioLegend cat #763102).

TGF-β may refer to an unmodified TGF-β polypeptide, a modified TGF-β polypeptide, a TGF-β variant, a TGF-β mutant; or a fragment thereof or a functional fragment thereof. Unmodified polypeptides, modified polypeptides, mutants, variants, and fragments are described herein. In some embodiments, TGF-β refers to a polypeptide comprising an amino acid sequence that is at least about 75% identical to SEQ ID NO: 20 or SEQ ID NO: 22. For example, TGF-β may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 20 or SEQ ID NO: 22.

In some embodiments, TGF-β refers to a fragment of a TGF-β polypeptide. Generally, a TGF-β fragment contains fewer amino acids than a full-length mature TGF-β. For example, a TGF-β fragment may include a portion of the mature human TGF-β polypeptide (e.g., a portion of amino acids 30-390 of SEQ ID NO: 20; a portion of amino acids 279-390 of SEQ ID NO: 20), or a portion of the mature mouse TGF-β polypeptide (e.g., a portion of amino acids 29-390 of SEQ ID NO: 22; a portion of amino acids 279-390 of SEQ ID NO: 22). An example full-length mature human TGF-β is 112 amino acids in length. Accordingly, fragments of human TGF-β may be 111 amino acids in length or shorter. An example full-length mature mouse TGF-β is 112 amino acids in length. Accordingly, fragments of mouse TGF-β may be 111 amino acids in length or shorter.

In some embodiments, TGF-β refers to a functional fragment of a TGF-β polypeptide. Any suitable method for assessing the activity of TGF-β and functional fragments of TGF-β may be used to determine whether a TGF-β fragment is a functional fragment. For example, one assay for assessing TGF-β activity is a cell proliferation inhibition assay. An example cell proliferation inhibition assay measures the extent to which TGF-β inhibits the proliferation of mouse HT-2 cells induced by recombinant mouse IL-4. In some embodiments, a functional fragment of TGF-β is a fragment that exhibits at least 50% of the activity of a TGF-β standard or a full-length mature TGF-β. For example, a functional fragment of TGF-β is a fragment that exhibits at least 50%, at least 60%, at least 70%, at least 80%, at least 90%, at least 95%, or more of the activity of a TGF-β standard or a full-length mature TGF-β.

### GM-CSF

Certain methods provided herein include use of granulocyte-macrophage colony-stimulating factor (GM-CSF) as a stimulant or co-stimulant. Granulocyte-macrophage colony-stimulating factor (also referred to as GM-CSF, colony stimulating factor 2, CSF2, CSF-α, Pluripoietin-α, Eosinophil colony stimulating factor (Eo-CSF), burst promoting activity (BPA)) plays a role in signaling emergency hemopoiesis (predominantly myelopoiesis) in response to infection, including the production of granulocytes and macrophages in the bone marrow and their maintenance, survival, and functional activation at sites of injury or insult. The receptor for GM-CSF is a heterodimer that comprises a major binding subunit (GMRα) and a major signaling subunit (βc). The receptor subunits are coexpressed on the surface of leukocytes, with pc being expressed at lower levels than GMRα. Certain nonhemopoietic cell types also have been reported to express the GM-CSF receptor and to respond to GM-CSF stimulation in vitro.

Any suitable GM-CSF, or functional fragment, or modified version thereof, may be used in conjunction with the methods described herein (e.g., as a co-stimulant with IL-40; as a stimulant prior to IL-40 exposure). In some embodiments, GM-CSF is a human GM-CSF. An example human GM-CSF nucleic acid sequence is provided herein as SEQ ID NO: 25 (full mRNA sequence provided as GENBANK Accession No. NM_000758), and an example human GM-CSF amino acid sequence is provided herein as SEQ ID NO: 24 (GENBANK Accession No. NP_000749.2). In some embodiments, GM-CSF is a mouse GM-CSF. An example mouse GM-CSF nucleic acid sequence is provided herein as SEQ ID NO: 27 (full mRNA sequence provided as GENBANK Accession No. NM_009969), and an example mouse GM-CSF amino acid sequence is provided herein as SEQ ID NO: 26 (GENBANK Accession No. NP_034099.2).

GM-CSF may refer to a precursor GM-CSF polypeptide (includes the signal peptide) or a mature GM-CSF polypeptide (excludes the signal peptide). In some embodiments, GM-CSF is a precursor GM-CSF polypeptide (e.g., a precursor human GM-CSF polypeptide comprising amino acids 1-144 of SEQ ID NO: 24; a precursor mouse GM-CSF polypeptide comprising amino acids 1-141 of SEQ ID NO: 26). In some embodiments, a GM-CSF polypeptide is a mature GM-CSF polypeptide (e.g., a mature human GM-CSF polypeptide comprising amino acids 18-144 of SEQ ID NO: 24; a mature mouse GM-CSF polypeptide comprising amino acids 18-141 of SEQ ID NO: 26).

In some embodiments, GM-CSF is a recombinant GM-CSF polypeptide. A recombinant GM-CSF polypeptide typically is a GM-CSF polypeptide encoded by DNA (i.e., GM-CSF nucleic acid sequence) that has been cloned in a vector or system that supports expression of the DNA and translation of messenger RNA. In some embodiments, GM-CSF is a recombinant human GM-CSF polypeptide (rhGM-CSF). In some embodiments, GM-CSF is a recombinant mouse GM-CSF polypeptide (rmGM-CSF). In some embodiments, GM-CSF is a commercially available recombinant human GM-CSF (e.g., BioLegend cat #572902). In some embodiments, GM-CSF is a commercially available recombinant mouse GM-CSF (e.g., BioLegend cat #576302).

GM-CSF may refer to an unmodified GM-CSF polypeptide, a modified GM-CSF polypeptide, a GM-CSF variant, a GM-CSF mutant; or a fragment thereof or a functional fragment thereof. Unmodified polypeptides, modified polypeptides, mutants, variants, and fragments are described herein. In some embodiments, GM-CSF refers to a polypeptide comprising an amino acid sequence that is at least about 75% identical to SEQ ID NO: 24 or SEQ ID NO: 26. For example, GM-CSF may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 24 or SEQ ID NO: 26.

In some embodiments, GM-CSF refers to a fragment of a GM-CSF polypeptide. Generally, a GM-CSF fragment contains fewer amino acids than a full-length mature GM-CSF. For example, a GM-CSF fragment may include a portion of the mature human GM-CSF polypeptide (i.e., a portion of amino acids 18-144 of SEQ ID NO: 24), or a portion of the mature mouse GM-CSF polypeptide (i.e. a portion of amino acids 18-141 of SEQ ID NO: 26). An example full-length mature human GM-CSF is 127 amino acids in length. Accordingly, fragments of human GM-CSF may be 126 amino acids in length or shorter. An example full-length mature mouse GM-CSF is 124 amino acids in length. Accordingly, fragments of mouse GM-CSF may be 123 amino acids in length or shorter.

In some embodiments, GM-CSF refers to a functional fragment of a GM-CSF polypeptide. Any suitable method for assessing the activity of GM-CSF and functional fragments of GM-CSF may be used to determine whether a GM-CSF fragment is a functional fragment. For example, one assay for assessing GM-CSF activity is a cell proliferation assay. An example cell proliferation assay determines EC₅₀ of GM-CSF by dose-dependent stimulation of TF-1 cell proliferation. In some embodiments, a functional fragment of GM-CSF is a fragment that exhibits at least 50% of the activity of a GM-CSF standard (e.g., 1st WHO International Standard for Human GM-CSF (NIBSC code: 88/646)) or a full-length mature GM-CSF. For example, a functional fragment of GM-CSF is a fragment that exhibits at least 50%, at least 60%, at least 70%, at least 80%, at least 90%, at least 95%, or more of the activity of a GM-CSF standard or a full-length mature GM-CSF.

### M-CSF

Certain methods provided herein include use of macrophage colony-stimulating factor (M-CSF) as a stimulant or co-stimulant. Macrophage colony-stimulating factor (also referred to as M-CSF, CSF1, CSF-1, MCSF) was first characterized as a glycoprotein that induces monocyte and macrophage colony formation from precursors in murine bone marrow cultures. M-CSF is constitutively present at biologically active concentrations in human serum. It binds CD14+ monocytes and promotes the survival/proliferation of human peripheral blood monocytes. In addition, M-CSF enhances inducible monocyte functions including phagocytic activity, microbial killing, cytotoxicity for tumor cells as well as synthesis of inflammatory cytokines such as IL-1, TNFα, and IFN-γ in monocytes. M-CSF induces RANKL production in mature human osteoclasts; consequently, M-CSF is a potent stimulator of mature osteoclast resorbing activity. Also, M-CSF induces VEGF in human monocytes in human tumors; high levels of M-CSF, mononuclear phagocytes, and VEGF are associated with poor prognosis in patients with cancer. High levels of M-CSF may be associated with different pathologies such as pulmonary fibrosis and atherosclerosis. M-CSF binds to its receptor M-CSFR, and this receptor is shared by a second ligand, IL-34. Human M-CSF and IL-34 exhibit cross-species specificity—both bind to human and mouse M-CSF receptors.

Any suitable M-CSF, or functional fragment, or modified version thereof, may be used in conjunction with the methods described herein (e.g., as a co-stimulant with IL-40; as a stimulant prior to IL-40 exposure). In some embodiments, M-CSF is a human M-CSF. An example human M-CSF nucleic acid sequence is provided herein as SEQ ID NO: 29 (full mRNA sequence provided as GENBANK Accession Nos. NM_172212.2 and NM_172212.3), and an example human M-CSF amino acid sequence is provided herein as SEQ ID NO: 28 (GENBANK Accession No. NP_757351.2). In some embodiments, M-CSF is a mouse M-CSF. An example mouse M-CSF nucleic acid sequence is provided herein as SEQ ID NO: 31 (full mRNA sequence provided as GENBANK Accession No. NM_001113530.1), and an example mouse M-CSF amino acid sequence is provided herein as SEQ ID NO: 30 (GENBANK Accession No. NP_001107002.1).

M-CSF may refer to a precursor M-CSF polypeptide (includes the signal peptide) or a mature M-CSF polypeptide (excludes the signal peptide). In some embodiments, M-CSF is a precursor M-CSF polypeptide (e.g., a precursor human M-CSF polypeptide comprising amino acids 1-190 of SEQ ID NO: 28; a precursor human M-CSF polypeptide comprising amino acids 1-450 of SEQ ID NO: 28; a precursor human M-CSF polypeptide comprising amino acids 1-554 of SEQ ID NO: 28; a precursor mouse M-CSF polypeptide comprising amino acids 1-262 of SEQ ID NO: 30; a precursor mouse M-CSF polypeptide comprising amino acids 1-552 of SEQ ID NO: 30). In some embodiments, a M-CSF polypeptide is a mature M-CSF polypeptide (e.g., a mature human M-CSF polypeptide comprising amino acids 33-190 of SEQ ID NO: 28; a mature human M-CSF polypeptide comprising amino acids 33-450 of SEQ ID NO: 28; a mature human M-CSF polypeptide comprising amino acids 33-554 of SEQ ID NO: 28; a mature mouse M-CSF polypeptide comprising amino acids 33-262 of SEQ ID NO: 30; a mature mouse M-CSF polypeptide comprising amino acids 33-552 of SEQ ID NO: 30).

In some embodiments, M-CSF is a recombinant M-CSF polypeptide. A recombinant M-CSF polypeptide typically is an M-CSF polypeptide encoded by DNA (i.e., M-CSF nucleic acid sequence) that has been cloned in a vector or system that supports expression of the DNA and translation of messenger RNA. In some embodiments, M-CSF is a recombinant human M-CSF polypeptide (rhM-CSF). In some embodiments, M-CSF is a recombinant mouse M-CSF polypeptide (rmM-CSF). In some embodiments, M-CSF is a commercially available recombinant human M-CSF (e.g., BioLegend cat #574802). In some embodiments, M-CSF is a commercially available recombinant mouse M-CSF (e.g., BioLegend cat #576402).

M-CSF may refer to an unmodified M-CSF polypeptide, a modified M-CSF polypeptide, an M-CSF variant, an M-CSF mutant; or a fragment thereof or a functional fragment thereof. Unmodified polypeptides, modified polypeptides, mutants, variants, and fragments are described herein. In some embodiments, M-CSF refers to a polypeptide comprising an amino acid sequence that is at least about 75% identical to SEQ ID NO: 28 or SEQ ID NO: 30. For example, M-CSF may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 28 or SEQ ID NO: 30.

In some embodiments, M-CSF refers to a fragment of an M-CSF polypeptide. Generally, an M-CSF fragment contains fewer amino acids than a full-length mature M-CSF. For example, an M-CSF fragment may include a portion of the mature human M-CSF polypeptide (e.g., a portion of amino acids 33-190 of SEQ ID NO: 28; a portion of amino acids 33-450 of SEQ ID NO: 28; a portion of amino acids 33-554 of SEQ ID NO: 28), or a portion of the mature mouse M-CSF polypeptide (e.g., a portion of amino acids 33-262 of SEQ ID NO: 30; a portion of amino acids 33-552 of SEQ ID NO: 30). An example full-length mature human M-CSF is 158 amino acids in length. Accordingly, fragments of human M-CSF may be 157 amino acids in length or shorter. An example full-length mature mouse M-CSF is 230 amino acids in length. Accordingly, fragments of mouse M-CSF may be 229 amino acids in length or shorter.

In some embodiments, M-CSF refers to a functional fragment of an M-CSF polypeptide. Any suitable method for assessing the activity of M-CSF and functional fragments of M-CSF may be used to determine whether an M-CSF fragment is a functional fragment. For example, one assay for assessing M-CSF activity is a cell proliferation assay. An example cell proliferation assay determines EC₅₀ of M-CSF by dose-dependent stimulation of M-NFS60 cell proliferation. In some embodiments, a functional fragment of M-CSF is a fragment that exhibits at least 50% of the activity of an M-CSF standard or a full-length mature M-CSF. For example, a functional fragment of M-CSF is a fragment that exhibits at least 50%, at least 60%, at least 70%, at least 80%, at least 90%, at least 95%, or more of the activity of an M-CSF standard or a full-length mature M-CSF.

### IFN-α

Certain methods provided herein include use of interferon alpha (IFN-α) as a stimulant or co-stimulant. Interferon alpha also may be referred to as IFN-α, IFN-α2, IFN-alpha 2B, IFN-alphaA, IFNA2, or IFNA2B. Interferons are divided into type I, II, and III. Type I IFNs (IFN-α and IFN-β) are most abundant in number, distribution, and expression. Also, they are highly conserved among mammals in both structure and function. IFN-α2 has been used in the treatment of cancer such as bladder cancer, hepatocellular carcinoma, and leukemia. IFN-α2 augments the suppressed immune functions in patients with head and neck squamous cell carcinoma (HNSCC). IFN-α2 initiated T and NK cell mediated cytotoxicity of tumor cells through IFNγ dependent and independent mechanisms. IFN-α2 enhances suppressed T cell cytotoxicity by stimulation of the perforin-granzyme B system (IFNγ dependent). Also, IFN-α2 induces the expression of perforin-granzyme B in NK cells (NK mediated cytotoxicity, IFNγ independent). IFN-α2 may be an immunostimulator and may impact the clinical outcome in tongue squamous cell carcinoma patients. IFN-α had been used in the treatment of chronic hepatitis C (CHC); nevertheless, IFN-α is relatively unstable and requires frequent parenteral administration. Pegylation of IFN-α, polyethylene glycol (PEG)-IFN-α, reduces in vitro activity but increase the stability and plasma half-life of IFN-α; therefore, PEG-IFN-α has replaced IFN-α in CHC treatment.

Any suitable IFN-α, or functional fragment, or modified version thereof, may be used in conjunction with the methods described herein (e.g., as a co-stimulant with IL-40; as a stimulant prior to IL-40 exposure). In some embodiments, IFN-α is a human IFN-α. An example human IFN-α nucleic acid sequence is provided herein as SEQ ID NO: 33 (full mRNA sequence provided as GENBANK Accession No. NM_000605), and an example human IFN-α amino acid sequence is provided herein as SEQ ID NO: 32 (GENBANK Accession No. NP_000596.2). In some embodiments, IFN-α is a mouse IFN-α. An example mouse IFN-α nucleic acid sequence is provided herein as SEQ ID NO: 35 (full mRNA sequence provided as GENBANK Accession No. NM_206870), and an example mouse IFN-α amino acid sequence is provided herein as SEQ ID NO: 34 (GENBANK Accession No. NP_996753.1).

IFN-α may refer to a precursor IFN-α polypeptide (includes the signal peptide) or a mature IFN-α polypeptide (excludes the signal peptide). In some embodiments, IFN-α is a precursor IFN-α polypeptide (e.g., a precursor human IFN-α polypeptide comprising amino acids 1-188 of SEQ ID NO: 32; a precursor mouse IFN-α polypeptide comprising amino acids 1-190 of SEQ ID NO: 34). In some embodiments, an IFN-α polypeptide is a mature IFN-α polypeptide (e.g., a mature human IFN-α polypeptide comprising amino acids 24-188 of SEQ ID NO: 32; a mature mouse IFN-α polypeptide comprising amino acids 24-190 of SEQ ID NO: 34).

In some embodiments, IFN-α is a recombinant IFN-α polypeptide. A recombinant IFN-α polypeptide typically is an IFN-α polypeptide encoded by DNA (i.e., IFN-α nucleic acid sequence) that has been cloned in a vector or system that supports expression of the DNA and translation of messenger RNA. In some embodiments, IFN-α is a recombinant human IFN-α polypeptide (rhIFN-α). In some embodiments, IFN-α is a recombinant mouse IFN-α polypeptide (rmIFN-α). In some embodiments, IFN-α is a commercially available recombinant human IFN-α (e.g., BioLegend cat #592702). In some embodiments, IFN-α is a commercially available recombinant mouse IFN-α (e.g., BioLegend cat #752802).

IFN-α may refer to an unmodified IFN-α polypeptide, a modified IFN-α polypeptide, an IFN-α variant, an IFN-α mutant; or a fragment thereof or a functional fragment thereof. Unmodified polypeptides, modified polypeptides, mutants, variants, and fragments are described herein. In some embodiments, IFN-α refers to a polypeptide comprising an amino acid sequence that is at least about 75% identical to SEQ ID NO: 32 or SEQ ID NO: 34. For example, IFN-α may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 32 or SEQ ID NO: 34.

In some embodiments, IFN-α refers to a fragment of an IFN-α polypeptide. Generally, an IFN-α fragment contains fewer amino acids than a full-length mature IFN-α. For example, an IFN-α fragment may include a portion of the mature human IFN-α polypeptide (e.g., a portion of amino acids 24-188 of SEQ ID NO: 32), or a portion of the mature mouse IFN-α polypeptide (e.g., a portion of amino acids 24-190 of SEQ ID NO: 34). An example full-length mature human IFN-α is 165 amino acids in length. Accordingly, fragments of human IFN-α may be 164 amino acids in length or shorter. An example full-length mature mouse IFN-α is 167 amino acids in length. Accordingly, fragments of mouse IFN-α may be 166 amino acids in length or shorter.

In some embodiments, IFN-α refers to a functional fragment of an IFN-α polypeptide. Any suitable method for assessing the activity of IFN-α and functional fragments of IFN-α may be used to determine whether an IFN-α fragment is a functional fragment. For example, one assay for assessing IFN-α activity is a cytopathic effect inhibition assay.

For example, specific activity of IFN-α may be determined in a cytopathic effect inhibition assay using the EMC virus on L929 or A549 cells. In some embodiments, a functional fragment of IFN-α is a fragment that exhibits at least 50% of the activity of an IFN-α standard or a full-length mature IFN-α. For example, a functional fragment of IFN-α is a fragment that exhibits at least 50%, at least 60%, at least 70%, at least 80%, at least 90%, at least 95%, or more of the activity of an IFN-α standard or a full-length mature IFN-α.

### IL-1β

Certain methods provided herein include use of interleukin 1 beta (IL-1β) as a stimulant or co-stimulant. Interleukin 1 beta also may be referred to as IL-1β, catabolin, preinterleukin 1 beta, or pro-interleukin-1-beta. IL-1β in humans and mice does not encode a typical signal peptide and, as a result, newly synthesized pro-IL-1β accumulates within the cytoplasm of activated monocytes and macrophages. Conversion of the inactive pro-IL-1 to its mature form requires the proteolytic action of IL-1β-converting enzyme (ICE), also termed caspase-1. Secretion of mature IL-1β from LPS-activated monocytes/macrophages is not a constitutive process. These cells encounter a secondary stimulus that specifically activates the posttranslational processing events. Moreover, owing to its pro-inflammatory nature, IL-1β is regarded as a tumor-promoting cytokine. Enhanced tumor metastasis and angiogenesis has been observed under the influence of IL-1β. IL-1β is able to facilitate tumor progression in murine models of lung cancer. In addition, upregulation of metastasis and tumor angiogenesis by IL-1β has been associated with increased activity of matrix metalloproteinases and expression of the pro-angiogenic molecule hepatocyte growth factor.

Any suitable IL-1β, or functional fragment, or modified version thereof, may be used in conjunction with the methods described herein (e.g., as a co-stimulant with IL-40; as a stimulant prior to IL-40 exposure). In some embodiments, IL-1β is a human IL-1β. An example human IL-1β nucleic acid sequence is provided herein as SEQ ID NO: 37 (full mRNA sequence provided as GENBANK Accession No. NM_000576), and an example human IL-1β amino acid sequence is provided herein as SEQ ID NO: 36 (GENBANK Accession No. NP_000567.1). In some embodiments, IL-1β is a mouse IL-1β. An example mouse IL-1β nucleic acid sequence is provided herein as SEQ ID NO: 39 (full mRNA sequence provided as GENBANK Accession No. NM_008361), and an example mouse IL-1β amino acid sequence is provided herein as SEQ ID NO: 38 (GENBANK Accession No. NP_032387.1).

IL-1β may refer to a precursor IL-1β polypeptide (includes the propeptide) or a mature IL-1β polypeptide (excludes the propeptide). In some embodiments, IL-1β is a precursor IL-1β polypeptide (e.g., a precursor human IL-1β polypeptide comprising amino acids 1-269 of SEQ ID NO: 36; a precursor mouse IL-1β polypeptide comprising amino acids 1-269 of SEQ ID NO: 38). In some embodiments, an IL-1β polypeptide is a mature IL-1β polypeptide (e.g., a mature human IL-1β polypeptide comprising amino acids 117-269 of SEQ ID NO: 36; a mature mouse IL-1β polypeptide comprising amino acids 118-269 of SEQ ID NO: 38).

In some embodiments, IL-1β is a recombinant IL-1β polypeptide. A recombinant IL-1β polypeptide typically is an IL-1β polypeptide encoded by DNA (i.e., IL-1β nucleic acid sequence) that has been cloned in a vector or system that supports expression of the DNA and translation of messenger RNA. In some embodiments, IL-1β is a recombinant human IL-1β polypeptide (rhIL-1β). In some embodiments, IL-1β is a recombinant mouse IL-1β polypeptide (rmIL-1β). In some embodiments, IL-1β is a commercially available recombinant human IL-1β (e.g., BioLegend cat #579402). In some embodiments, IL-1β is a commercially available recombinant mouse IL-1β (e.g., BioLegend cat #575102).

IL-1β may refer to an unmodified IL-1β polypeptide, a modified IL-1β polypeptide, an IL-1β variant, an IL-1β mutant; or a fragment thereof or a functional fragment thereof.

Unmodified polypeptides, modified polypeptides, mutants, variants, and fragments are described herein. In some embodiments, IL-1β refers to a polypeptide comprising an amino acid sequence that is at least about 75% identical to SEQ ID NO: 36 or SEQ ID NO: 38. For example, IL-1β may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 36 or SEQ ID NO: 38.

In some embodiments, IL-1β refers to a fragment of an IL-1β polypeptide. Generally, an IL-1β fragment contains fewer amino acids than a full-length mature IL-1β. For example, an IL-1β fragment may include a portion of the mature human IL-1β polypeptide (e.g., a portion of amino acids 117-269 of SEQ ID NO: 36), or a portion of the mature mouse IL-1β polypeptide (e.g., a portion of amino acids 118-269 of SEQ ID NO: 38). An example full-length mature human IL-1β is 153 amino acids in length. Accordingly, fragments of human IL-1β may be 152 amino acids in length or shorter. An example full-length mature mouse IL-1β is 152 amino acids in length. Accordingly, fragments of mouse IL-1β may be 151 amino acids in length or shorter.

In some embodiments, IL-1β refers to a functional fragment of an IL-1β polypeptide. Any suitable method for assessing the activity of IL-1β and functional fragments of IL-13 may be used to determine whether an IL-1β fragment is a functional fragment. For example, one assay for assessing IL-1β activity is a cell proliferation assay. For example, the ED₅₀ of IL-1β may be determined by dose dependent stimulation of D10.G4.1 cell proliferation. In some embodiments, a functional fragment of IL-1β is a fragment that exhibits at least 50% of the activity of an IL-1β standard or a full-length mature IL-1β. For example, a functional fragment of IL-1β is a fragment that exhibits at least 50%, at least 60%, at least 70%, at least 80%, at least 90%, at least 95%, or more of the activity of an IL-1β standard or a full-length mature IL-13.

### IL-13

Certain methods provided herein include use of interleukin 13 (IL-13) as a stimulant or co-stimulant. Interleukin 13 also may be referred to as IL-13, ALRH, BHR1, P600, IL-13, MGC116786, MGC116788, MGC116789, NC30, or IL13. Human IL-13 was initially cloned from cDNA libraries of activated T cells. IL-13 is an immunoregulatory cytokine secreted predominantly by activated T(H)2 cells, and it is a mediator in the pathogenesis of allergic inflammation. IL-13 shares many functional properties with IL-4, and they share a common receptor subunit, the alpha subunit of the IL-4 receptor (IL-4Ralpha). IL-13 mediates its effects by interacting with a complex receptor system comprised of IL-4Ralpha and two IL-13 binding proteins, IL-13Ralpha1 and IL-13Ralpha2. Ligation of the IL-13 receptor complex results in signaling via the insulin receptor substrate (IRS)-1 and 2 and STAT-6 pathways. IL-13, like IL-4, is a cytokine produced by T(H)2 type helper T cells in response to signaling through the T cell antigen receptor and by mast cells and basophils upon cross-linkage of the high-affinity receptor for immunoglobulin E (IgE). IL-13 has been implicated in airway hypersensitivity and mucus hypersecretion, inflammatory bowel disease, and parasitic nematode expulsion.

Any suitable IL-13, or functional fragment, or modified version thereof, may be used in conjunction with the methods described herein (e.g., as a co-stimulant with IL-40; as a stimulant prior to IL-40 exposure). In some embodiments, IL-13 is a human IL-13. An example human IL-13 nucleic acid sequence is provided herein as SEQ ID NO: 41 (full mRNA sequence provided as GENBANK Accession No. X69079), and an example human IL-13 amino acid sequence is provided herein as SEQ ID NO: 40 (GENBANK Accession No. CAA48823.1 or P35225.2). Another example human IL-13 amino acid sequence is provided as GENBANK Accession No. NP_002179.2. In some embodiments, IL-13 is a mouse IL-13. An example mouse IL-13 nucleic acid sequence is provided herein as SEQ ID NO: 43 (full mRNA sequence provided as GENBANK Accession No. NM_008355), and an example mouse IL-13 amino acid sequence is provided herein as SEQ ID NO: 42 (GENBANK Accession No. NP_032381.1).

IL-13 may refer to a precursor IL-13 polypeptide (includes the signal peptide) or a mature IL-13 polypeptide (excludes the signal peptide). In some embodiments, IL-13 is a precursor IL-13 polypeptide (e.g., a precursor human IL-13 polypeptide comprising amino acids 1-132 of SEQ ID NO: 40; a precursor human IL-13 polypeptide comprising amino acids 1-146 of SEQ ID NO: 40; a precursor mouse IL-13 polypeptide comprising amino acids 1-131 of SEQ ID NO: 42). In some embodiments, an IL-13 polypeptide is a mature IL-13 polypeptide (e.g., a mature human IL-13 polypeptide comprising amino acids 21-132 of SEQ ID NO: 40; a mature mouse IL-13 polypeptide comprising amino acids 26-131 of SEQ ID NO: 42).

In some embodiments, IL-13 is a recombinant IL-13 polypeptide. A recombinant IL-13 polypeptide typically is an IL-13 polypeptide encoded by DNA (i.e., IL-13 nucleic acid sequence) that has been cloned in a vector or system that supports expression of the DNA and translation of messenger RNA. In some embodiments, IL-13 is a recombinant human IL-13 polypeptide (rhIL-13). In some embodiments, IL-13 is a recombinant mouse IL-13 polypeptide (rmIL-13). In some embodiments, IL-13 is a commercially available recombinant human IL-13 (e.g., BioLegend cat #571102). In some embodiments, IL-13 is a commercially available recombinant mouse IL-13 (e.g., BioLegend cat #575902).

IL-13 may refer to an unmodified IL-13 polypeptide, a modified IL-13 polypeptide, an IL-13 variant, an IL-13 mutant; or a fragment thereof or a functional fragment thereof. Unmodified polypeptides, modified polypeptides, mutants, variants, and fragments are described herein. In some embodiments, IL-13 refers to a polypeptide comprising an amino acid sequence that is at least about 75% identical to SEQ ID NO: 40 or SEQ ID NO: 42. For example, IL-13 may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 40 or SEQ ID NO: 42.

In some embodiments, IL-13 refers to a fragment of an IL-13 polypeptide. Generally, an IL-13 fragment contains fewer amino acids than a full-length mature IL-13. For example, an IL-13 fragment may include a portion of the mature human IL-13 polypeptide (e.g., a portion of amino acids 21-132 of SEQ ID NO: 40), or a portion of the mature mouse IL-13 polypeptide (e.g., a portion of amino acids 26-131 of SEQ ID NO: 42). An example full-length mature human IL-13 is 112 amino acids in length. Accordingly, fragments of human IL-13 may be 111 amino acids in length or shorter. An example full-length mature mouse IL-13 is 106 amino acids in length. Accordingly, fragments of mouse IL-13 may be 105 amino acids in length or shorter.

In some embodiments, IL-13 refers to a functional fragment of an IL-13 polypeptide. Any suitable method for assessing the activity of IL-13 and functional fragments of IL-13 may be used to determine whether an IL-13 fragment is a functional fragment. For example, one assay for assessing IL-13 activity is a cell proliferation assay. For example, the ED₅₀ of IL-13 may be determined by dose dependent stimulation of TF-1 cell proliferation. In some embodiments, a functional fragment of IL-13 is a fragment that exhibits at least 50% of the activity of an IL-13 standard or a full-length mature IL-13. For example, a functional fragment of IL-13 is a fragment that exhibits at least 50%, at least 60%, at least 70%, at least 80%, at least 90%, at least 95%, or more of the activity of an IL-13 standard or a full-length mature IL-13.

### TNF-α

Certain methods provided herein include use of tumor necrosis factor alpha (TNF-α) as a stimulant or co-stimulant. Tumor necrosis factor alpha (TNF-α) also may be referred to as cachectin, necrosin, macrophage cytotoxic factor (MCF), differentiation inducing factor (DIF), or TNFSF2. TNF-α is released from macrophages, monocytes, neutrophils, T-cells (principally CD4+), NK-cells and many transformed cell lines. Soluble homotrimeric TNF-α is released from cells by proteolysis of the integral membrane precursor form of TNF-α. TNF-α binding to some TNF-α receptors induces apoptosis and depending on cell type, receptor expression, and signal transduction status can induce other responses. TNF-α is involved in the inflammatory response.

Any suitable TNF-α, or functional fragment, or modified version thereof, may be used in conjunction with the methods described herein (e.g., as a co-stimulant with IL-40; as a stimulant prior to IL-40 exposure). In some embodiments, TNF-α is a human TNF-α. An example human TNF-α nucleic acid sequence is provided herein as SEQ ID NO: 45 (full mRNA sequence provided as GENBANK Accession No. NM_000594), and an example human TNF-α amino acid sequence is provided herein as SEQ ID NO: 44 (GENBANK Accession No. NP_000585.2). In some embodiments, TNF-α is a mouse TNF-α. An example mouse TNF-α nucleic acid sequence is provided herein as SEQ ID NO: 47 (full mRNA sequence provided as GENBANK Accession No. NM_013693), and an example mouse TNF-α amino acid sequence is provided herein as SEQ ID NO: 46 (GENBANK Accession No. NP_038721.1).

TNF-α may refer to a precursor TNF-α polypeptide (integral membrane precursor form) or a mature TNF-α polypeptide (soluble homotrimeric form). In some embodiments, TNF-α is a precursor TNF-α polypeptide (e.g., a precursor human TNF-α polypeptide comprising amino acids 1-233 of SEQ ID NO: 44; a precursor mouse TNF-α polypeptide comprising amino acids 1-235 of SEQ ID NO: 46). In some embodiments, a TNF-α polypeptide is a mature TNF-α polypeptide (e.g., a mature human TNF-α polypeptide comprising amino acids 77-233 of SEQ ID NO: 44; a mature mouse TNF-α polypeptide comprising amino acids 80-235 of SEQ ID NO: 46).

In some embodiments, TNF-α is a recombinant TNF-α polypeptide. A recombinant TNF-α polypeptide typically is a TNF-α polypeptide encoded by DNA (i.e., TNF-α nucleic acid sequence) that has been cloned in a vector or system that supports expression of the DNA and translation of messenger RNA. In some embodiments, TNF-α is a recombinant human TNF-α polypeptide (rhTNF-α). In some embodiments, TNF-α is a recombinant mouse TNF-α polypeptide (rmTNF-α). In some embodiments, TNF-α is a commercially available recombinant human TNF-α (e.g., BioLegend cat #570102). In some embodiments, TNF-α is a commercially available recombinant mouse TNF-α (e.g., BioLegend cat #575202).

TNF-α may refer to an unmodified TNF-α polypeptide, a modified TNF-α polypeptide, a TNF-α variant, a TNF-α mutant; or a fragment thereof or a functional fragment thereof. Unmodified polypeptides, modified polypeptides, mutants, variants, and fragments are described herein. In some embodiments, TNF-α refers to a polypeptide comprising an amino acid sequence that is at least about 75% identical to SEQ ID NO: 44 or SEQ ID NO: 46. For example, TNF-α may refer to a polypeptide comprising an amino acid sequence that is at least about 75%, 80%, 85%, 90%, 91%, 92%, 93%, 94%, 95%, 96%, 97%, 98%, or 99% identical to SEQ ID NO: 44 or SEQ ID NO: 46.

In some embodiments, TNF-α refers to a fragment of a TNF-α polypeptide. Generally, a TNF-α fragment contains fewer amino acids than a full-length mature TNF-α. For example, a TNF-α fragment may include a portion of the mature human TNF-α polypeptide (e.g., a portion of amino acids 77-233 of SEQ ID NO: 44), or a portion of the mature mouse TNF-α polypeptide (e.g., a portion of amino acids 80-235 of SEQ ID NO: 46). An example full-length mature human TNF-α is 157 amino acids in length. Accordingly, fragments of human TNF-α may be 156 amino acids in length or shorter. An example full-length mature mouse TNF-α is 156 amino acids in length. Accordingly, fragments of mouse TNF-α may be 155 amino acids in length or shorter.

In some embodiments, TNF-α refers to a functional fragment of a TNF-α polypeptide. Any suitable method for assessing the activity of TNF-α and functional fragments of TNF-α may be used to determine whether a TNF-α fragment is a functional fragment. For example, one assay for assessing TNF-α activity is a cytotoxicity assay. For example, the ED₅₀ of TNF-α may be determined by dose-dependent cytotoxicity of L929 cells stimulated with actinomycin D. In some embodiments, a functional fragment of TNF-α is a fragment that exhibits at least 50% of the activity of a TNF-α standard (e.g., 3rd WHO International Standard for Human TNF-α (NIBSC code: 12/154)) or a full-length mature TNF-α. For example, a functional fragment of TNF-α is a fragment that exhibits at least 50%, at least 60%, at least 70%, at least 80%, at least 90%, at least 95%, or more of the activity of a TNF-α standard or a full-length mature TNF-α.

### Cells

Certain methods described herein include stimulating a cell or population of cells (e.g., with IL-40; with a stimulant; with IL-40+a co-stimulant). Cells may be obtained from a subject and/or a cellular source, or may be obtained as an established cell line. A cellular source may include a population of embryonic stem (ES) cells, induced pluripotent stem cells (iPSCs), and the like. Cells may be isolated from an embryo or a stem cell culture derived from an embryo. Cells may be isolated from an induced pluripotent stem cell (iPSC) culture. Cells may be obtained from a subject in a variety of manners (e.g., harvested from living tissue, such as a biopsy, plucked hair follicles, body fluids like urine or body-cavity fluids, or isolated from circulation). A subject may include any animal, including but not limited to any mammal, such as mouse, rat, canine, feline, bovine, equine, porcine, non-human primate and human. In certain embodiments, a subject is a human. In some embodiments, a subject is an animal or human that has gestated longer than an embryo in a uterine environment and often is a post-natal human or a post-natal animal (e.g., neonatal human, neonatal animal, adult human or adult animal). A subject sometimes is a juvenile animal, juvenile human, adult animal or adult human.

In some embodiments, cells are isolated from a sample from a subject. An isolated cell refers to a cell that has been separated from a component of its original environment (e.g., separated from a host and/or purified from a sample), and thus is altered by human intervention (e.g., “by the hand of man”) from its original environment. A sample can include any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), biopsy sample or tissue biopsy, buccal swab, celocentesis sample, washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated.

In some embodiments, cells comprise normal, healthy cells (e.g., cells that are not diseased). In some embodiments, cells comprise cells that are genetically altered. In some embodiments, cells comprise cells that are not genetically altered. In some embodiments, cells comprise diseased cells. Diseased cells may include cells from a subject carrying disease-causing mutation(s). Diseased cells may include cells from abnormal tissue, such as from a neoplasia, a hyperplasia, a malignant tumor or a benign tumor. In certain embodiments, diseased cells include cells that are not tumor cells. In certain embodiments, diseased cells may include cells isolated from circulation (e.g., circulating tumor cells (CTCs)) of a subject. In certain embodiments, diseased cells may include cells isolated from bodily samples such as, for example, urine, semen, stool (feces), and the like.

In some embodiments, cells comprise primary cells. Primary cells generally are taken directly from living tissue, such as a biopsy, plucked hair follicles, bodily samples such as a stool sample, body fluids like urine, semen or body-cavity fluids, or isolated from circulation. In certain instances, primary cells have not been passaged. In certain instances, primary cells have been passaged one time. Primary cells may be isolated from differentiated tissue. Typically, primary cells have been freshly isolated, for example, through tissue digestion and plated. Primary cells may or may not be frozen and then thawed at a later time. In addition, the tissue from which the primary cells are isolated may or may not have been frozen or preserved in some other manner immediately prior to processing. Typically, cells are no longer primary cells after the cells have been passaged more than once. Cells passaged once or more and immediately frozen after passaging are also not considered as primary cells when thawed. In certain embodiments, cells are initially primary cells and become non-primary cells after passaging. In some embodiments, cells are maintained or proliferated in cell culture after the cells are isolated from differentiated tissue and prior to use in methods described herein.

In some embodiments, cells comprise non-primary cells, such as cells from an established cell line, transformed cells, thawed cells from a previously frozen collection, and the like. Any suitable cell line may be used in conjunction with the methods described herein. Examples of established cell lines include, for example, THP-1 (acute myeloid leukemia), DU145 (prostate cancer), H295R (adrenocortical cancer), HeLa (cervical cancer), KBM-7 (chronic myelogenous leukemia), LNCaP (prostate cancer), MCF-7 (breast cancer), MDA-MB-468 (breast cancer), PC3 (prostate cancer), SaOS-2 (bone cancer), SH-SY5Y (neuroblastoma, cloned from a myeloma), T-47D (breast cancer), U87 (glioblastoma), Vero (African green monkey Chlorocebus kidney epithelial cell line), MC3T3 (embryonic calvarium), GH3 (pituitary tumor), PC12 (pheochromocytoma), CHO (Chinese hamster ovary), MDCK (kidney epithelial), A6 (kidney epithelial), and AB9. In some embodiments, cells comprise THP-1 cells.

In some embodiments, cells comprise immune cells. Immune cells may include, for example, lymphocytes, leukocytes, agranulocytes, monocytes, macrophages, B cells, dendritic cells, granulocytes, neutrophils, innate lymphoid cells (ILC), megakaryocytes, myeloid-derived suppressor cells (MDSC), natural killer cells (NK cells), platelets, red blood cells (RBC), T cells, mast cells, eosinophils, basophils, and thymocytes. In some embodiments, cells comprise, or are derived from, peripheral blood mononuclear cells (PBMCs) which may include, for example, T cells, B cells, natural killer cells, and monocytes.

In some embodiments, cells comprise monocytes. Monocytes are a type of leukocyte, or white blood cell, and can differentiate into macrophages and myeloid lineage dendritic cells. Monocytes are a part of the vertebrate innate immune system and can influence the process of adaptive immunity. There are at least three subclasses of monocytes in human blood, which may be characterized according to certain markers. For example, the classical monocyte is characterized by high level expression of the CD14 cell surface receptor (CD14++CD16− monocyte); the non-classical monocyte shows low level expression of CD14 and additional co-expression of the CD16 receptor (CD14+CD16++monocyte); and the intermediate monocyte is characterized by high level expression of CD14 and low level expression of CD16 (CD14++CD16+ monocytes).

In some embodiments, cells comprise macrophages. Macrophages are a type of white blood cell that engulf and digest foreign proteins and other substances, cellular debris, microbes, and cancer cells. Macrophages may be referred to as phagocytes, histiocytes, Kupffer cells, alveolar macrophages, or microglia, and may be found in nearly all body tissues. Macrophages are involved in nonspecific defense (innate immunity) and can help initiate specific defense mechanisms (adaptive immunity) by recruiting other immune cells such as lymphocytes. Macrophages also have an anti-inflammatory role and can decrease immune reactions through the release of cytokines. Macrophages that encourage inflammation are often referred to as M1 macrophages, and macrophages that decrease inflammation and encourage tissue repair are often referred to as M2 macrophages. Macrophages are produced by the differentiation of monocytes. Macrophages may be identified by their specific expression of proteins such as CD14, CD40, CD11b, CD64, F4/80, EMR1, lysozyme M, MAC-1/MAC-3 and CD68 using flow cytometry and/or immunohistochemical staining. Dysfunctional macrophages may cause diseases such as chronic granulomatous disease that results in frequent infections.

In some embodiments, cells comprise non-immune cells. Non-immune cells may include, for example, epithelial cells (e.g., cells lining body cavities), cells derived from the central nervous system (e.g., nerve cells, neurons, neuroglial cells), stromal cells (e.g., connective tissue cells, fibroblasts, pericytes), stem cells (e.g., embryonic stem cells, adult stem cells), muscle cells (e.g., skeletal, cardiac, smooth), cartilage cells (e.g., chondrocytes), bone cells (e.g., osteoblasts, osteoclasts, osteocytes, lining cells), skin cells (e.g., keratinocytes, melanocytes, merkel cells, langerhans cells), endothelial cells (e.g., cells lining blood vessels), fat cells (e.g., white adipocytes, brown adipocytes), and sex cells (spermatozoa, ova).

In some embodiments, cells comprise epithelial cells. An epithelial cell, or epithelium, typically refers to a cell or cells that line hollow organs, as well as those that make up glands and the outer surface of the body. Epithelial cells can comprise squamous epithelial cells, columnar epithelial cells, adenomatous epithelial cells or transitional epithelial cells. Epithelial cells can be arranged in single layers or can be arranged in multiple layers, depending on the organ and location, and may comprise keratinocyte (KE) epithelial cells or non-keratinocyte (NKE) epithelial cells.

Keratinocytes form the squamous epithelium that is found at anatomic sites such as the skin, ocular surface, oral mucosa, esophagus and cervix. Keratinocytes terminally differentiate into flat, highly keratinized, non-viable cells that help protect against the environment and infection by forming a protective barrier. Examples of keratinocyte epithelial cells include, but are not limited to, dermal keratinocyte, ocular epithelial cells, corneal epithelial cells, oral mucosal epithelial cells, esophagus epithelial cells, and cervix epithelial cells.

Non-keratinocyte (NKE) epithelial cells form the epithelium of the body such as found in the breast, prostate, liver, respiratory tract, retina and gastrointestinal tract. NKE cells typically differentiate into functional, viable cells which function, for example, in absorption and/or secretion. These cells typically do not form highly keratinized structures characteristic of squamous epithelial cells. Examples of NKE cells include, but are not limited to, prostate cells, mammary cells, hepatocytes, liver epithelial cells, biliary epithelial cells, gall bladder cells, pancreatic islet cells, pancreatic beta cells, pancreatic ductal epithelial cells, pulmonary epithelial cells, airway epithelial cells, nasal epithelial cells, kidney cells, bladder cells, urethral epithelial cells, stomach epithelial cells, large intestinal epithelial cells, small intestinal epithelial cells, testicular epithelial cells, ovarian epithelial cells, fallopian tube epithelial cells, thyroid cells, parathyroid cells, adrenal cells, thymus cells, pituitary cells, glandular cells, amniotic epithelial cells, retinal pigmented epithelial cells, sweat gland epithelial cells, sebaceous epithelial cells and hair follicle cells.

### Cytokine, Chemokine, and Growth Factor Production

Certain methods described herein include measuring production of one or more cytokines and/or chemokines. Certain methods described herein include measuring production of one or more cytokines, chemokines, and/or growth factors.

Cytokines generally refer to small proteins (generally about 5-20 kDa) that are involved in cell signaling, and a release of cytokines can have an effect on the behavior of cells nearby. Cytokines are often considered as immunomodulating agents and may be involved in autocrine signaling, paracrine signaling and endocrine signaling. Cytokines may include chemokines, interferons, interleukins, lymphokines, monokines, colony stimulating factors, and tumor necrosis factors. Cytokines may be produced by immune cells (e.g., monocytes, macrophages, B lymphocytes, T lymphocytes, and mast cells), endothelial cells, fibroblasts, and stromal cells.

In some embodiments, a method herein comprises measuring production by a cell of one or more cytokines. In some embodiments, a method herein comprises measuring production by a cell of one or more interleukins. In some embodiments, a method herein comprises measuring production by a cell of one or more IL-6 family cytokines. In some embodiments, a method herein comprises measuring production by a cell of one or more IL-1 family cytokines. A method herein may include measuring production of one or more cytokines provided in Table 1 below. Also provided in Table 1 are corresponding human genes and human receptors, however, cytokines, genes and receptors listed in Table 1 are not limited to human cytokines, genes and receptors. In some embodiments, a method herein comprises measuring production by a cell of a cytokine that binds to one or more receptors provided in Table 1.

TABLE 1
Examples of cytokines

| Cytokine | Human gene | Human receptor(s) |
|---|---|---|
| IL-6 family | | |
| IL-6 | Interleukin 6 | IL6R (CD126), GP130, sIL6R |
| IL-11 | Interleukin 11 | IL11R |
| Oncostatin M | Oncostatin M | LIPR/IL6ST/OSMR |
| Ciliary neurotrophic | Ciliary Neurotrophic | CNTFR |
| factor | Factor | |
| NNT-1/BSF-3/CLC | Novel Neurotrophin- | CNTFR |
| | 1/B-Cell Stimulating | |
| | Factor-3 | |
| | Cardiotrophin-Like | |
| | Cytokine Factor 1 | |
| Cardiotrophin-1 | Cardiotrophin 1 | GP130/LIFR |
| Leukemia inhibitory | Leukemia inhibitory | GP130/LIFR |
| factor | factor | |
| IL-27 | Interleukin 27 | IL27RA/GP130 |
| IL-31 | Interleukin 31 | IL31RA/OSMR |
| IL-1 family | | |
| IL-1RA | Interleukin 1 | IL1R |
| | Receptor Antagonist | |
| IL-1α | Interleukin 1 Alpha | IL1R |
| IL-1β | Interleukin 1 Beta | |
| IL-18 | Interleukin 18 | IL18RA/IL18RB |
| IL-33 | Interleukin 33 | ST2/IL-1 R4 / IL-1 RAcP |
| IL-36α | Interleukin 36 alpha | IL-1 Rrp2 / IL-1 RAcP |
| IL-36β | Interleukin 36 beta | IL-1 Rrp2 / IL-1 RAcP |
| IL-36γ | Interleukin 36 gamma | IL-1 Rrp2 / IL-1 RAcP |
| IL-36Ra | Interleukin 36 | IL-1 Rrp2 / IL-1 RAcP |
| | Receptor Antagonist | |
| IL-37 | Interleukin 37 | IL18RA |
| IL-38 | Interleukin 38 | IL-1 RI/ IL1 Rrp2 |

Chemokines generally refer to a sub-family of cytokines (signaling proteins secreted by cells). Chemokines can induce directed chemotaxis in nearby responsive cells, and may be referred to as chemotactic cytokines. Chemokines are small (generally about 8-10 kDa) and typically have four cysteine residues in conserved locations for forming their 3-dimensional shape. Certain chemokines are pro-inflammatory and can be induced during an immune response to recruit cells of the immune system to a site of infection, and certain chemokines are homeostatic and are involved in controlling the migration of cells during normal processes of tissue maintenance or development. Chemokines may be classified into subfamilies (i.e., C, CX3C, CC and CXC), and exert their biological effects by interacting with G protein-linked transmembrane receptors (chemokine receptors), that are found on the surface of target cells.

In some embodiments, a method herein comprises measuring production by a cell of one or more chemokines. In some embodiments, a method herein comprises measuring production by a cell of one or more C-family chemokines. In some embodiments, a method herein comprises measuring production by a cell of one or more CX3C-family chemokines. In some embodiments, a method herein comprises measuring production by a cell of one or more CC-family chemokines. In some embodiments, a method herein comprises measuring production by a cell of one or more CXC-family chemokines. A method herein may include measuring production of one or more chemokines provided in Table 2 below. Also provided in Table 2 are corresponding human genes and human receptors, however, chemokines, genes and receptors herein are not limited to human chemokines, genes and receptors. In some embodiments, a method herein comprises measuring production by a cell of a chemokine that binds to one or more receptors provided in Table 2.

TABLE 2
Examples of chemokines

| Chemokine | Human gene | Human receptor(s) |
|---|---|---|
| C Family | | |
| XCL1 | XCL1 | XCR1 |
| XCL2 | XCL2 | XCR1 |
| CX3C Family | | |
| CX3CL1 | CX3CL1 | CXCR1 |
| CC Family | | |
| CCL1 | CCL1 | CCR8, DARC |
| CCL2 | CCL2 | CCR2, CCR4, CCR11, D6, DARC |
| CCL3 | CCL3 | CCR1, CCR4, CCR5, D6 |
| CCL3L1 | CCL3L1 | CCR1, CCR3, CCR5, D6 |
| CCL3L3 | CCL3L3 | CCR1, CCR3, CCR5 |
| CCL4 | CCL4 | CCR1, CCR5, CCR8, D6 |
| CCL4L1 | CCL4L1 | CCR1, CCR5 |
| CCL4L2 | CCL4L2 | CCR1, CCR5 |
| CCL5 | CCL5 | CCR1, CCR3, CCR4, CCR5, D6, |
| | | DARC |
| CCL61 | CCL23 | |
| CCL7 | CCL7 | CCR1, CCR2, CCR3, D6, DARC |
| CCL8 | CCL8 | CCR1, CCR2, CCR3, CCR5, CCR11, |
| | | D6, DARC |
| CCL11 | CCL11 | CCR3, CCR5, D6, DARC |
| CCL12 | | |
| CCL13 | CCL13 | CCR1, CCR2, CCR3, CCR5, CCR11, |
| | | D6, DARC |
| CCL14 | CCL14 | CCR1, CCR3, CCR5, D6, DARC |
| CCL15 | CCL15 | CCR1, CCR3 |
| CCL16 | CCL16 | CCR1, CCR2, CCR5, CCR8, DARC, |
| | | H4 |
| CCL17 | CCL17 | CCR4, CCR8, D6, DARC |
| CCL18 | CCL18 | CCR8, PITPNM3, DARC |
| CCL19 | CCL19 | CCR7, CCR11, CCRL2/ CRAM A/B |
| CCL20 | CCL20 | CCR6 |
| CCL21 | CCL21 | CCR7, CCR11 |
| CCL22 | CCL22 | CCR4, D6 |
| CCL23 | CCL23 | CCR1, FPRL-1 |
| CCL24 | CCL24 | CCR3 |
| CCL25 | CCL25 | CCR9, CCR11 |
| CCL26 | CCL26 | CCR3, CX3CR1 |
| CCL27 | CCL27 | CCR10 |
| CCL28 | CCL28 | CCR3, CCR10 |
| CXC Family | | |
| CXCL1 | CXCL1 | CXCR2, DARC |
| CXCL2 | CXCL2 | CXCR2, DARC |
| CXCL3 | CXCL3 | CXCR2, DARC |
| CXCL4 | PF4 | CXCR3, CXCR3B, DARC |
| CXCL4L1 | PF4V1 | CXCR3, CXCR3B |
| CXCL5 | CXCL5 | CXCR2, DARC |
| CXCL6 | CXCL6 | CXCR1, CXCR2, DARC |
| CXCL7 | PPBP | CXCR1, CXCR2 |
| CXCL8 | IL-8 | CXCR1, CXCR2, DARC |
| CXCL9 | CXCL9 | CXCR3, CXCR3B, DARC |
| CXCL10 | CXCL10 | CXCR3, CXCR3B, DARC |
| CXCL11 | CXCL11 | CXCR3, CXCR3B, CXCR7, DARC |
| CXCL12 | CXCL12 | CXCR4, CXCR7 |
| CXCL13 | CXCL13 | CXCR3, CXCR5, DARC |
| CXCL14 | CXCL14 | Unknown |
| CXCL15 | | |
| CXCL16 | CXCL16 | CXCR6 |
| CXCL17 | CXCL17 | Unknown |
| LIX | | |

Growth factors generally refer to a substance capable of stimulating cellular growth, proliferation, healing, cellular differentiation, and the like. A growth factor may be a protein or a hormone (e.g., steroid hormone). In certain instances, cytokines may be categorized as growth factors. Growth factors may include, for example, erythropoietin, platelet-derived growth factor (PDGF), PDGF-AA, and vascular endothelial growth factor (VEGF).

In some embodiments, a method herein comprises measuring production by a cell of one or more cytokines and/or chemokines chosen from CCL2, CCL3, CCL4, CCL5, CCL11, CCL17, CCL20, CXCL1, CXCL5, CXCL8, CXCL9, CXCL10, CXCL11, IL-1RA, and IL-6. In some embodiments, a method herein comprises measuring production by a cell of one or more cytokines, chemokines and/or growth factors chosen from CCL2, CCL3, CCL4, CCL5, CCL11, CCL17, CCL20, CXCL1, CXCL5, CXCL8, CXCL9, CXCL10, CXCL11, IL-1RA, IL-6, erythropoietin, PDGF-AA, and VEGF. In some embodiments, a method herein comprises measuring production by a cell of one or more cytokines and/or chemokines chosen from CCL2, CCL3, CCL4, CCL5, CCL11, CXCL8, CXCL10, and IL-1RA. In some embodiments, a method herein comprises measuring production by a cell of one or more cytokines, chemokines, and/or growth factors chosen from CCL2, CCL3, CCL4, CCL5, CCL11, CXCL8, CXCL10, IL-1RA, erythropoietin, PDGF-AA, and VEGF. In some embodiments, a method herein comprises measuring production by a cell of CCL2. In some embodiments, a method herein comprises measuring production by a cell of CCL3. In some embodiments, a method herein comprises measuring production by a cell of CCL4. In some embodiments, a method herein comprises measuring production by a cell of CCL5. In some embodiments, a method herein comprises measuring production by a cell of CCL11. In some embodiments, a method herein comprises measuring production by a cell of CCL17. In some embodiments, a method herein comprises measuring production by a cell of CCL20. In some embodiments, a method herein comprises measuring production by a cell of CXCL1. In some embodiments, a method herein comprises measuring production by a cell of CXCL5. In some embodiments, a method herein comprises measuring production by a cell of CXCL8. In some embodiments, a method herein comprises measuring production by a cell of CXCL9. In some embodiments, a method herein comprises measuring production by a cell of CXCL10. In some embodiments, a method herein comprises measuring production by a cell of CXCL11. In some embodiments, a method herein comprises measuring production by a cell of IL-1RA. In some embodiments, a method herein comprises measuring production by a cell of IL-6. In some embodiments, a method herein comprises measuring production by a cell of erythropoietin. In some embodiments, a method herein comprises measuring production by a cell of PDGF-AA. In some embodiments, a method herein comprises measuring production by a cell of VEGF.

Cytokine, chemokine, and/or growth factor production may be measured using any suitable method, apparatus or machine for measuring protein secretion and/or DNA expression (e.g., mRNA). For example, cytokine, chemokine, and/or growth factor production may be measured by immunoassay (e.g., enzyme-linked immunosorbent assay (ELISA), protein immunoprecipitation, immunoelectrophoresis, Western blot, protein immunostaining), spectrometry (e.g., high-performance liquid chromatography (HPLC), liquid chromatography-mass spectrometry (LC/MS)), flow cytometry, quantitative polymerase chain reaction (qPCR), gel electrophoresis, a luminometer, a fluorometer, a spectrophotometer, a suitable gene-chip or microarray analysis, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof. Example immunoassays include LEGEND MAX™ ELISA kits with pre-coated plates (BioLegend), Macrophage/Microglia LEGENDplex™ panels (BioLegend), and Chemokine Inflammatory LEGENDplex™ panels (BioLegend).

### Methods for Assessing IL-40 Activity

Provided herein are methods for assessing IL-40 activity. Methods for assessing IL-40 activity generally are performed ex vivo (i.e., outside of an organism) or in vitro (i.e., performed or taking place in a test tube, culture dish, or other vessel outside of an organism). The terms ex vivo and in vitro may be used interchangeably, and typically are used in conjunction with methods performed in an artificial environment or artificial system. Methods herein generally include contacting a cell with IL-40 or IL-40 and a co-stimulant, and such contacting is typically performed ex vivo/in vitro (e.g., in cell culture).

In some embodiments, a method comprises contacting a cell with IL-40, measuring production of one or more cytokines, chemokines, and/or growth factors described herein, and detecting the activity of IL-40 according to the cytokine production. In some embodiments, detecting the activity of IL-40 comprises comparing the cytokine/chemokine/growth factor production under test conditions (IL-40 stimulation) to cytokine/chemokine/growth factor production under control conditions (no IL-40 stimulation). In some embodiments, detecting the activity of IL-40 comprises comparing cytokine/chemokine/growth factor production under test conditions to cytokine/chemokine/growth factor production in a standard curve (e.g., cytokine/chemokine/growth factor production measured for a plurality of different amounts of active IL-40). An active IL-40 may include an IL-40 standard (e.g., an unmodified full-length mature IL-40; a full-length mature IL-40 comprising activity-enhancing modifications). In some embodiments, detecting the activity of IL-40 comprises comparing cytokine/chemokine/growth factor production under test conditions to cytokine/chemokine/growth factor production measured for a control, thereby providing a comparison. A control may include, for example, cytokine/chemokine/growth factor production measured in the absence of IL-40. Typically, for a control, or for generating a standard curve, cytokine production is measured for the same cell (e.g., same cell-type, same cellular source, and/or same cell population) used for assessing IL-40 activity (test conditions). Typically, for a control, or for generating a standard curve, cytokine/chemokine/growth factor production can be a measured level of a cytokine/chemokine/growth factor, or measured levels of cytokines/chemokines/growth factors in a combination of cytokines, chemokines, growth factors; or cytokines and chemokines; or cytokines and growth factors; or chemokines and growth factors; or cytokines, chemokines and growth factors. Often, the level of the same cytokine/chemokine/growth factor, or the levels of the same cytokines, chemokines, or cytokines and chemokines, or cytokines and growth factors, or chemokines and growth factors, or cytokines, chemokines and growth factors in the same cytokine/chemokine/growth factor combination, is/are measured for test conditions and for control conditions or conditions for a standard curve.

In some embodiments, a method comprises contacting a cell with IL-40 and a co-stimulant (e.g., IFN-γ, GM-CSF, IFN-α, IL-1β, IL-4, IL-10, IL-13, M-CSF, TGF-β, TNF-α), measuring production of one or more cytokines, chemokines, and/or growth factors described herein, and detecting the activity of IL-40 according to the cytokine, chemokine, and/or growth factor production. In some embodiments, detecting the activity of IL-40 comprises comparing the cytokine/chemokine/growth factor production under test conditions (IL-40+co-stimulant) to cytokine/chemokine/growth factor production under control conditions (no IL-40, no co-stimulant, or no IL-40+co-stimulant). In some embodiments, detecting the activity of IL-40 comprises comparing cytokine/chemokine/growth factor production under test conditions to cytokine/chemokine/growth factor production in a standard curve (e.g., cytokine/chemokine/growth factor production measured for a plurality of different amounts of active IL-40 or active IL-40+co-stimulant). An active IL-40 may include an IL-40 standard (e.g., an unmodified full-length mature IL-40; a full-length mature IL-40 comprising activity-enhancing modifications). In some embodiments, detecting the activity of IL-40 comprises comparing cytokine/chemokine/growth factor production under test conditions to cytokine/chemokine/growth factor production measured for a control, thereby providing a comparison. A control may include, for example, cytokine/chemokine/growth factor production measured in the absence of IL-40; cytokine/chemokine/growth factor production measured in the absence of a co-stimulant; cytokine/chemokine/growth factor production measured in the absence of IL-40 and a co-stimulant. Typically, for a control, or for generating a standard curve, cytokine/chemokine/growth factor production is measured for the same cell (e.g., same cell-type, same cellular source, and/or same cell population) used for assessing IL-40 activity (test conditions). Typically, for a control, or for generating a standard curve, cytokine/chemokine/growth factor production can be a measured level of a cytokine/chemokine/growth factor, or measured levels of cytokines/chemokines/growth factors in a combination of cytokines, chemokines, growth factors, or cytokines and chemokines, or cytokines and growth factors, or chemokines and growth factors, or cytokines, chemokines, and growth factors. Often, the level of the same cytokine/chemokine/growth factor, or the levels of the same cytokines, chemokines, growth factors, or cytokines and chemokines, or cytokines and growth factors, or chemokines and growth factors, or cytokines, chemokines, and growth factors, in the same cytokine/chemokine/growth factor combination, is/are measured for test conditions and for control conditions or conditions for a standard curve.

In some embodiments, the production of one or more cytokines, chemokines, and/or growth factors under test conditions (i.e., under IL-40 stimulation or under IL-40+co-stimulant stimulation) is increased compared to the production under control conditions (i.e., absence of IL-40 or IL-40+co-stimulant). In some embodiments, production of one or more cytokines, chemokines, and/or growth factors under test conditions is increased by at least about 10% compared to the production under control conditions. For example, production of one or more cytokines, chemokines, and/or growth factors under test conditions may be increased by at least about 10%, 20%, 30%, 40%, 50%, 60%, 70%, 80%, 90%, 100%, 200%, 500%, 1000% or more compared to the production under control conditions. In some embodiments, production of one or more cytokines, chemokines, and/or growth factors under test conditions is increased by at least about 2-fold compared to the production under control conditions. For example, production of one or more cytokines, chemokines, and/or growth factors under test conditions may be increased by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more compared to the production under control conditions.

In some embodiments, a method comprises contacting a cell or population of cells with IL-40, detecting differentiation of the cell or differentiation of one or more cells in the population, and assessing the activity of IL-40 according to the differentiation. In some embodiments, a method comprises contacting a population of immune cells with IL-40, detecting differentiation of one or more immune cells in the population, and assessing the activity of IL-40 according to the differentiation. In some embodiments, a method comprises contacting a population of monocytes with IL-40, detecting monocyte to macrophage differentiation in the population, and assessing the activity of IL-40 according to the monocyte to macrophage differentiation. Macrophage differentiation may refer to any stage of macrophage differentiation including, but not limited to, M1, M2a, M2b, M2c, and M2d.

Monocyte to macrophage differentiation may be detected according to changes in cell morphology. For example, monocyte to macrophage differentiation may be characterized by attachment/adhesion to a culture plate, cells with a flattened and/or spreading appearance, cells with feelers/extensions/protrusions, and/or cells with irregular shape (compared to spherical or substantially spherical monocytes). In some embodiments, monocyte to macrophage differentiation under test conditions (i.e., under IL-40 stimulation or under IL-40+co-stimulant stimulation) is observed for a portion of cells in the population, compared to no or substantially no (e.g., less than 1%) monocyte to macrophage differentiation under control conditions (i.e., absence of IL-40 or IL-40+co-stimulant). For example, monocyte to macrophage differentiation under test conditions may be observed for at least about 5%, 10%, 20%, 30%, 40%, 50%, 60%, 70%, 80%, 90% or 100% of cells in the population. In some embodiments, monocyte to macrophage differentiation under IL-40 stimulation is observed for at least about 10% of cells in the population. In some embodiments, monocyte to macrophage differentiation under IL-40+co-stimulant stimulation is observed for at least about 30% of cells in the population.

Monocyte to macrophage differentiation may be detected according to changes in expression and/or production of one or more markers, cytokines, chemokines, and/or growth factors. In some embodiments, monocyte to macrophage differentiation is detected according to expression (presence or absence, increased, or decreased) of one or more markers (e.g., cell-surface markers). Expression of markers may be detected and/or quantified using any suitable method for detecting cell surface proteins (e.g., immunoassay (ELISA), flow cytometry) and/or mRNA expression (e.g., reverse transcriptase quantitative PCR). In some embodiments, monocyte to macrophage differentiation is detected according to production (presence or absence, increased, or decreased) of one or more cytokines, chemokines, and/or growth factors. Production of cytokines, chemokines, and/or growth factors may be detected and/or quantified using any suitable method for detecting secreted proteins (e.g., detection methods described herein). Presence, absence, increases, and decreases of markers, cytokines, chemokines, and/or growth factors may be determined by measuring levels of expression or production under test conditions (i.e., stimulation by IL-40 or stimulation by IL-40+co-stimulant) compared to levels of expression or production under control conditions (i.e., absence of IL-40 or absence of IL-40+co-stimulant).

Examples of markers useful for identifying monocyte to macrophage differentiation are provided in Table 3 below.

TABLE 3
Markers for monocytes/macrophages

| | Estimated | Estimated |
|---|---|---|
| | expression | expression |
| | level in | level in |
| Protein marker | monocytes | macrophages |
| CD14 | ++++/++ | ++ |
| Ly6C | ++++/++ | + |
| CD115 | ++++ | + |
| CD15s | ++ | + |
| CD33 | ++++ | ++ |
| CD44 | ++ | ++++ |
| CD81 | ++ | ++++ |
| CD49e | ++ | ++++ |
| CD18 | ++ | ++++ |
| CD11b | ++ | ++++ |
| CD54 (ICAM-1) | ++ | ++++ |
| CD11c | + | +++ |
| CD68 | ++ | ++++ |
| CD80 | ++ | ++++ |
| CD86 | ++ | ++++ |
| CD163 | ++ | ++++ |
| IL-1R | ++ | ++++ |
| CD200R | ++ | ++++ |

Examples of cytokines and chemokines useful for identifying monocyte to macrophage differentiation are provided in Table 4 below.

TABLE 4
Chemokines and cytokines having
increased secretion in macrophages

| Chemokines | Cytokines |
|---|---|
| CCL10 | TNF |
| CCL11 | IL-1 beta |
| CCL5 | IL-6 |
| CCL8 | IL-12 |
| CCL9 | IL-23 |
| CCL2 | IL-10 |
| CCL3 | TGF-beta |
| CCL4 | IL-1RA |
| CCL17 | IL-1 |
| CCL22 | TNF-alpha |
| CCL24 | IL-10 |
| CCL1 | |
| CCR2 | |
| CCL5 | |
| CXCL10 | |
| CXCL8 | |
| CXCL9 | |
| CXCL16 | |

In some embodiments, monocyte to macrophage differentiation is detected according to expression of one or more markers chosen from CD14, Ly6C, CD115, CD15s, CD33, CD44, CD81, CD49e, CD18, CD11b, CD54 (ICAM-1), CD11c, CD68, CD80, CD86, CD163, IL-1R, and CD200R. In some embodiments, monocyte to macrophage differentiation is detected according to increased expression of one or more markers chosen from CD44, CD81, CD49e, CD18, CD11b, CD54 (ICAM-1), CD11c, CD68, CD80, CD86, CD163, IL-1R, and CD200R. In some embodiments, monocyte to macrophage differentiation is detected according to decreased expression of one or more markers chosen from CD14, Ly6C, CD115, CD15s, and CD33. In some embodiments, monocyte to macrophage differentiation is detected according to increased production of one or more chemokines chosen from CCL10, CCL11, CCL5, CCL8, CCL9, CCL2, CCL3, CCL4, CCL17, CCL22, CCL24, CCL1, CCR2, CCL5, CXCL10, CXCL8, CXCL9, and CXCL16. In some embodiments, monocyte to macrophage differentiation is detected according to increased production of one or more cytokines chosen from TNF, IL-1 beta, IL-6, IL-12, IL-23, IL-10, TGF-beta, IL-1RA, IL-1, TNF-alpha, and IL-10.

### Methods for Inducing Cell Differentiation

Certain methods herein include inducing cell differentiation. Cell differentiation may be induced ex vivo or in vitro according to methods herein. In some embodiments, cell differentiation is induced by contacting a cell or population of cells with IL-40. Certain methods herein include inducing immune cell differentiation. In some embodiments, immune cell differentiation is induced by contacting an immune cell or a population of immune cells with IL-40. Certain methods herein include inducing monocyte to macrophage differentiation. In some embodiments, monocyte to macrophage differentiation is induced by contacting a monocyte or a population of monocytes with IL-40. Macrophage differentiation may refer to any stage of macrophage differentiation including, but not limited to, M1, M2a, M2b, M2c, and M2d.

Monocyte to macrophage differentiation may be detected according to changes in cell morphology. Methods for assessing changes in cell morphology are described herein. Monocyte to macrophage differentiation may be detected according to changes in expression and/or production of one or more markers, cytokines, chemokines, and/or growth factors. Markers, cytokines, chemokines, and/or growth factors useful for detecting monocyte to macrophage differentiation, and methods for detecting/quantifying their expression/production, are described herein.

### Kits

Provided in certain embodiments are kits. The kits may include any components and compositions described herein (e.g., IFN-γ, GM-CSF, IFN-α, IL-1β, IL-4, IL-10, IL-13, M-CSF, TGF-β, and/or TNF-α; one or more components for measuring cytokine, chemokine, and/or growth factor production, a cell or population of cells) useful for performing any of the methods described herein, in any suitable combination. In some embodiments, a kit further includes IL-40 (e.g., for use as a standard/control and/or for the user to generate a standard curve). IL-40 for testing may be provided by the user/purchaser of the kit. Kits may further include any reagents, buffers, or other components useful for carrying out any of the methods described herein. For example, a kit may include one or more binding molecules that immunospecifically bind to one or more cytokines, chemokines, and/or growth factors under binding conditions.

Components of a kit may be present in separate containers, or multiple components may be present in a single container. Suitable containers include a single tube (e.g., vial), a cell culture plate, one or more wells of a plate (e.g., a 6-well plate, a 12-well plate, a 24-well plate, a 96-well plate, a 384-well plate, and the like), and the like.

Kits may also comprise instructions for performing one or more methods described herein and/or a description of one or more components described herein. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### EXAMPLES

The examples set forth below illustrate certain embodiments and do not limit the technology.

### Example 1: Production of Cytokines and Chemokines by THP-1 Cells and Changes in Cell Morphology Under IL-40 Stimulation

In this Example, the production of certain cytokines and chemokines by THP-1 cells under IL-40 stimulation and IFN-γ+IL-40 co-stimulation was measured. Changes in cell morphology of THP-1 cells under IL-40 stimulation and IFN-γ+IL-40 co-stimulation also was observed.

### Materials and Methods

### Recombinants Proteins

The nucleic acid encoding the c17orf99 (chromosome 17 open reading frame 99) [Homo sapiens (human) NCBI Reference Sequence: NP_001156547], also known as IL-40, or UNQ464, was used to produce recombinant human protein IL-40 (rhIL-40). The protein contains an Fc fraction at the C terminus for recovery and purification purposes. The rhFc fraction from the IgG1 protein was used as a control. The recombinant proteins, rhFc, rhIL-40, IFN-γ (BioLegend cat #570202), IFN-α (BioLegend cat #592702), IL-4 (BioLegend cat #592702), IL-13 (BioLegend cat #571102), TGF-3 (BioLegend cat #580704), M-CSF (BioLegend cat #574802), GM-CSF (BioLegend cat #572902), IL-10 (BioLegend cat #715602), TNF-α (BioLegend cat #570102), and IL-1β (BioLegend cat #579402) were obtained from BioLegend.

### Cell Culture

The THP-1 cell line was cultured and maintained in RPMI-1640 medium supplemented with 10% fetal bovine serum (FBS), at 37° C. in a humidified atmosphere of 5% CO₂. To perform the stimulation experiments, THP-1 cells were cultured in RPMI-1640 medium supplemented with 1.1% FBS. 30,000 THP-1 cells were seeded in a 96-well plate, and stimulated with rhIL-40 (BioLegend, CA) or rhFc fraction (BioLegend, CA) at the same micromolar concentrations, in the presence or absence of IFN-γ (250 or 50 ng/mL), IFN-α, IL-4, Il-13, TGF-β, M-CSF, GM-CSF, IL-10, TNF-α, or IL-1β (50 ng/mL) for 24, 48, or 72 hours in a final volume of 200 μL.

Healthy human PBMCs were isolated from whole blood through density centrifugation. Cells were cultured in RPMI-1640 medium supplemented with 1.1% FBS. 30,000 PBMCs were seeded in a 96-well plate, and stimulated with rhIL-40 (BioLegend, CA) or rhFc fraction (control) at the same micromolar concentrations for 24 hours in a final volume of 200 μL.

### Cytokines and Chemokines Quantification

The concentration of CCL2, CCL3, CCL4, CCL5, CCL11, CCL17, CCL20, CXCL1, CXCL5, CXCL8, CXCL9, CXCL10, CXCL11, IL-12p70, IL-12p40, TNF-α, IL-6, IL-4, IL-10, IL-1β, Arginase, IL-1RA and IL-23, were measured in the cell culture supernatant using LEGEND MAX ELISA kits with pre-coated plates, or human Macrophage/Microglia and Chemokine Inflammatory LEGENDplex panels. The protocols were followed as suggested by the provider.

### Cell Phenotype

Changes in the morphology of THP-1 cells 24 hours after rhIL-40 stimulation were documented using a Keyence BZ-X700 microscope in a phase contrast mode and 10× lenses resolution.

### Data Analysis

The effect of different stimuli was normalized. For this purpose, the value in pg/mL of each molecule was divided by the value obtained under control conditions (unstimulated cells).

To quantify the effect of rhIL-40 or rhIL40+IFN-γ in the THP-1 monocytes morphology, the total cell number and cells attached to the plate surface were counted in several fields. The percentage of cells with morphological changes was determined using the following calculation:

(attached cell number×100%)/total cell number.

### Results

### CXCL8 Production

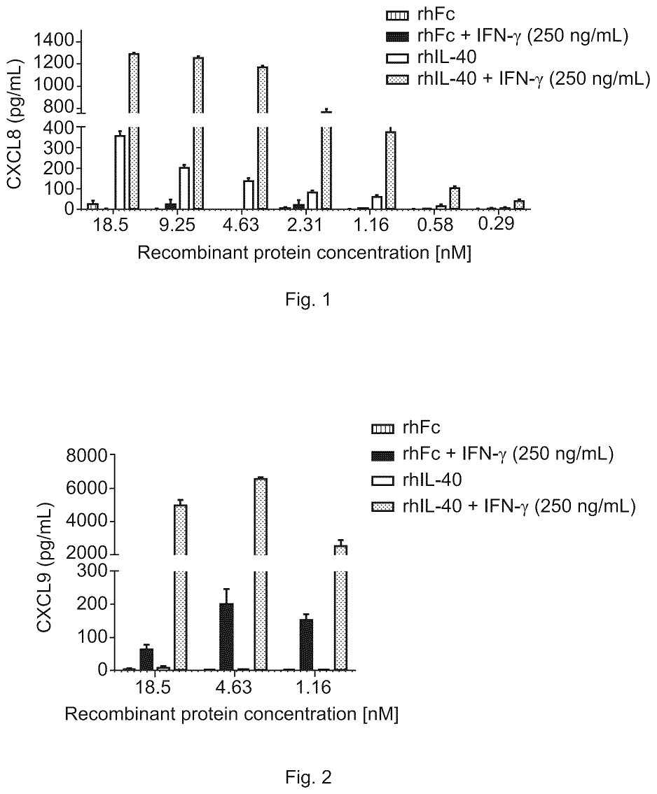

The production of CXCL8 was measured in cell supernatants from THP-1 cells stimulated with different concentrations of the recombinant proteins rhFc or rhIL-40 (0.29 μM, 0.58 μM, 1.16 μM, 2.31 μM, 4.63 μM, 9.25 μM, or 18.5 μM)±IFN-γ (250 ng/mL) for 24 hours, and the results are shown in FIG. 1. A dose dependent production of CXCL8 was observed when stimulating cells with rhIL-40 or rhIL-40+IFN-γ. A higher effect was observed after stimulation with rhIL-40+IFN-γ.

### CXCL9 Production

The production of CXCL9 was measured in cell supernatants from THP-1 cells stimulated with different concentrations of the recombinant proteins rhFc or rhIL-40 (1.16 μM, 4.63 μM, or 18.5 μM)±IFN-γ (250 ng/mL) for 24 hours, and the results are shown in FIG. 2. A significant increase in production of CXCL9 was observed when stimulating THP-1 cells with rhIL-40+IFN-γ. A lower effect in the CXCL9 secretion also was observed in cells stimulated with rhFc+IFN-γ.

### CCL2 Production

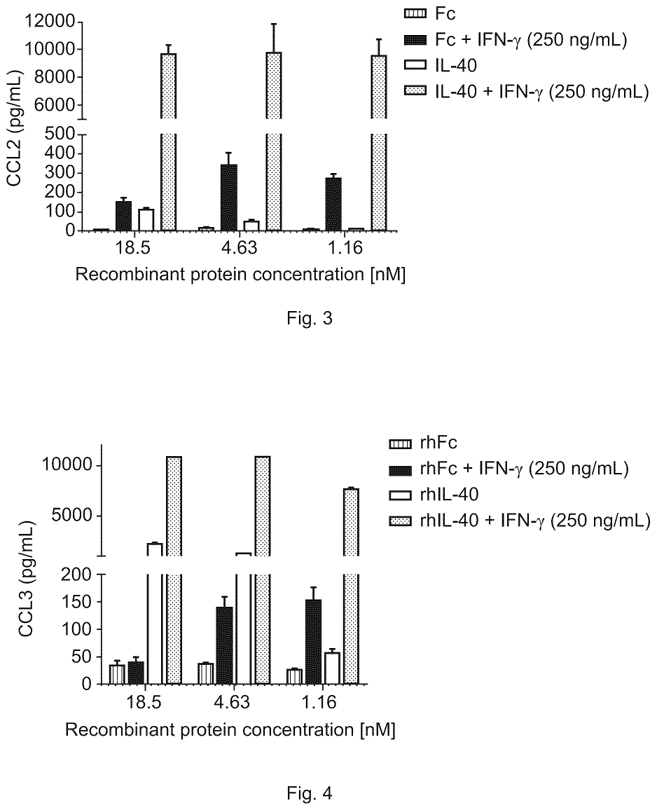

The production of CCL2 was measured in cell supernatants from THP-1 cells stimulated with different concentrations of the recombinant proteins rhFc or rhIL-40 (1.16 μM, 4.63 μM, or 18.5 μM)±IFN-γ (250 ng/mL) for 24 hours, and the results are shown in FIG. 3. A dose dependent production of CCL2 was observed when stimulating with rhIL-40. Maximum detection level of CCL2 was reached when stimulating cells with rhIL-40+IFN-γ, independent of the rhIL-40 concentration. A lower effect in CCL2 secretion also was observed in cells stimulated with rhFc+IFN-γ.

### CCL3 Production

The production of CCL3 was measured in cell supernatants from THP-1 cells stimulated with different concentrations of the recombinant proteins rhFc or rhIL-40 (1.16 μM, 4.63 μM, or 18.5 μM)±IFN-γ (250 ng/mL) for 24 hours, and the results are shown in FIG. 4. A dose dependent production of CCL3 was observed after stimulation with rhIL-40. A synergic effect was observed when stimulating with rhIL-40+IFN-γ. Similar effects were observed using the two top concentrations evaluated for rhIL-40. An effect in CCL3 secretion also was observed in cells stimulated with rhFc+IFN-γ.

### CCL4 Production

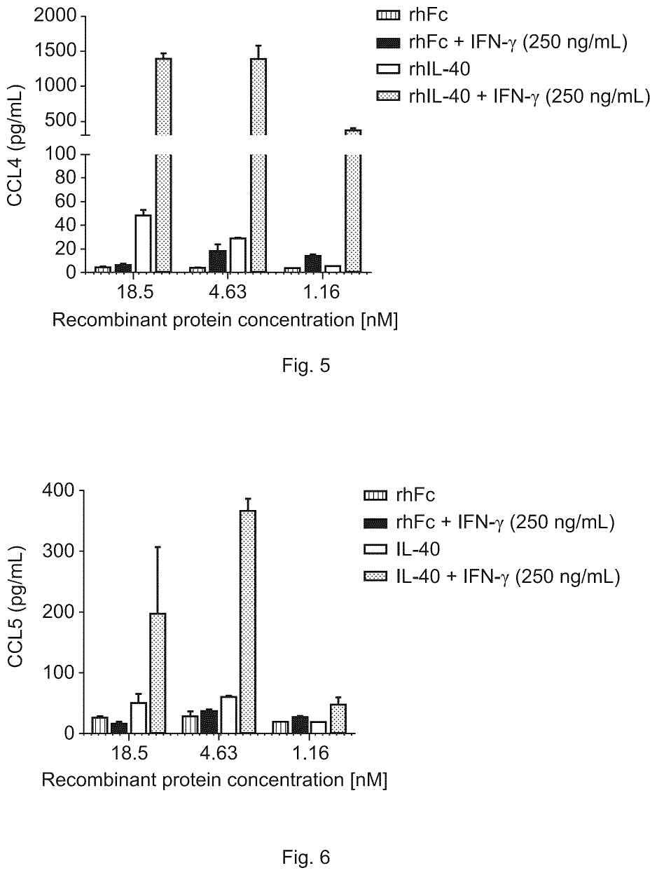

The production of CCL4 was measured in cell supernatants from THP-1 cells stimulated with different concentrations of the recombinant proteins rhFc or rhIL-40 (1.16 μM, 4.63 μM, or 18.5 μM)±IFN-γ (250 ng/mL) for 24 hours, and the results are shown in FIG. 5. A dose dependent production of CCL4 was observed when stimulating with rhIL-40. A synergic effect was observed when stimulating with rhIL-40+IFN-γ. Similar effects were observed using the two top concentrations evaluated for rhIL-40. An effect in the CCL4 secretion also was observed in cells stimulated with rhFc+IFN-γ.

### CCL5 Production

The production of CCL5 was measured in cell supernatants from THP-1 cells stimulated with different concentrations of the recombinant proteins rhFc or rhIL-40 (1.16 μM, 4.63 μM, or 18.5 μM)±IFN-γ (250 ng/mL) for 24 hours, and the results are shown in FIG. 6. An increase in the production of CCL5 was observed when stimulating with rhIL-40 or rhIL-40+IFN-γ. An effect in CCL5 secretion also was observed in cells stimulated with rhFc and rhFc+IFN-γ.

### CXCL10 Production

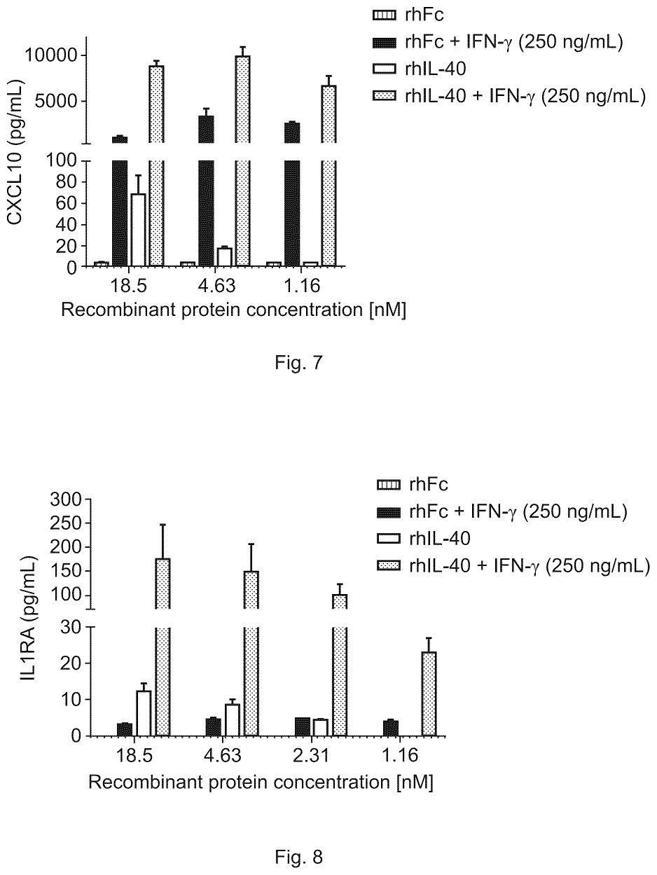

The production of CXCL10 was measured in cell supernatants from THP-1 cells stimulated with different concentrations of the recombinant proteins rhFc or rhIL-40 (1.16 μM, 4.63 μM, or 18.5 μM)±IFN-γ (250 ng/mL) for 24 hours, and the results are shown in FIG. 7. A significant increased production of CXCL10 was observed when stimulating THP-1 cells with rhIL40, rhFc+IFN-γ, or rhIL-40+IFN-γ. rhIL-40 alone was able to induce the production of CXCL10. The production of CXCL10 was exacerbated in presence of IFN-γ, showing higher production when combined with rhIL-40.

### IL1RA Production

The production of IL1RA was measured in cell supernatants from THP-1 cells stimulated with different concentrations of the recombinant proteins rhFc or rhIL-40 (0.58 μM, 2.31 μM, 4.63 μM, or 18.5 μM)±IFN-γ (250 ng/mL) for 24 hours, and the results are shown in FIG. 8. A dose dependent production of IL1 RA was observed when stimulating with rhIL-40 or rhIL-40+IFN-γ. A synergic effect was observed when stimulating with rhIL-40+IFN-γ. An effect in IL1RA secretion also was observed in cells stimulated with rhFc+IFN-γ.

### IL-6 Production

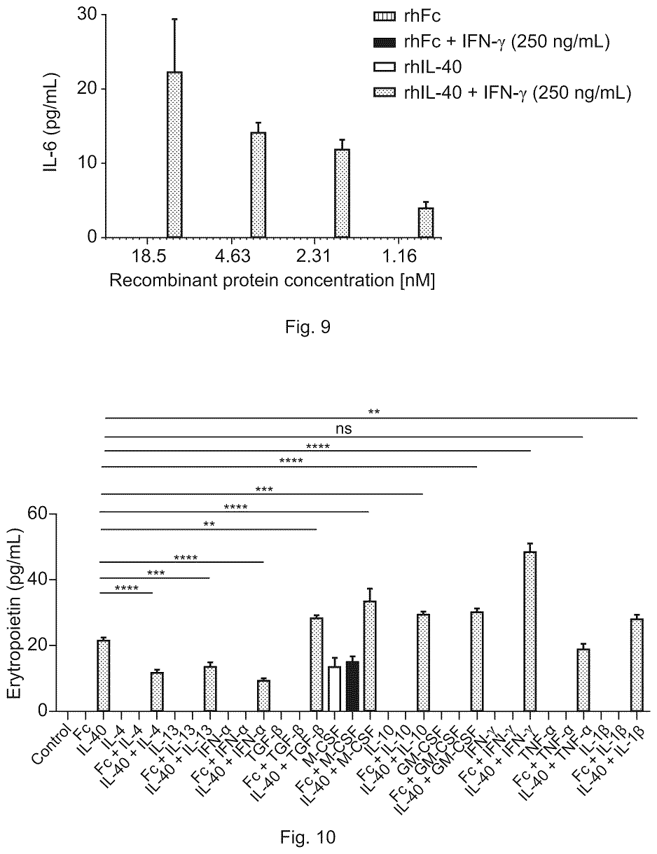

The production of IL-6 was measured in cell supernatants from THP-1 cells stimulated with different concentrations of the recombinant proteins rhFc or rhIL-40 (0.58 μM, 2.31 μM, 4.63 μM or 18.5 μM)±IFN-γ (250 ng/mL) for 24 hours, and the results are shown in FIG. 9. A significant increase in production of IL-6 was observed when stimulating THP-1 cells with rhIL-40+IFN-γ.

Similar to the effect in the production of chemokines and growth factors by THP-1 cells stimulated with rhIL40 and IFN-γ, an exacerbation in the production of several molecules was observed when cells were stimulated with rhIL40 and IL-10, GM-CSF, TNF-α, or IL-1β for 24 hours.

### Erythropoietin Production

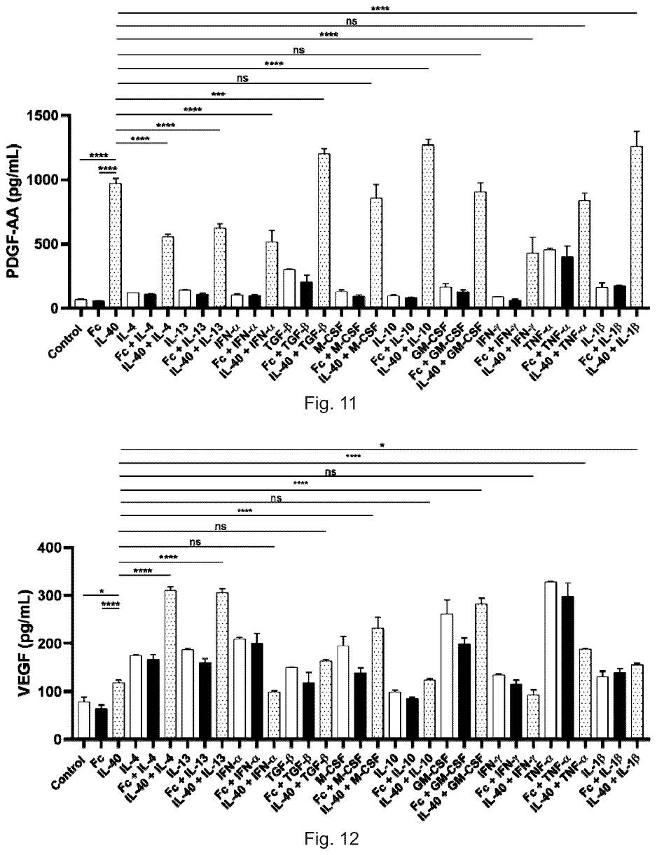

The production of erythropoietin was measured in cell supernatants from THP-1 cells stimulated with different concentrations of the recombinant proteins rhFc or rhIL-40 (4.63 μM μM)±50 ng/mL of IL-4, IL-13, IFN-α, TGF-β, M-CSF, IL-10, GM-CSF, IFN-γ, TNF-α, or IL-1β for 24 hours, and the results are shown in FIG. 10. A significant increase in production of erythropoietin was observed when stimulating THP-1 cells with rhIL-40, and its production was exacerbated when cells were co-stimulated with rhIL40 plus TGF-β, M-CSF, IL-10, GM-CSF, IFN-γ, or IL-1β.

### PDGF-AA Production

The production of PDGF-AA was measured in cell supernatants from THP-1 cells stimulated with different concentrations of the recombinant proteins rhFc or rhIL40 (4.63 μM)±50 ng/mL of IL-4, Il-13, IFN-α, TGF-β, M-CSF, IL-10, GM-CSF, IFN-γ, TNF-α, or IL-1β for 24 hours, and the results are shown in FIG. 11. A significant increase in production of PDGF-AA was observed when stimulating THP-1 cells with rhIL-40, and its production is exacerbated when cells are co-stimulated with rhIL40 plus IFN-γ, TGF-β, IL-10, or IL-1β.

### VEGF Production

The production of VEGF was measured in cell supernatants from THP-1 cells stimulated with different concentrations of the recombinant proteins rhFc or rhIL-40 (4.63 μM)±250 ng/mL of IL-4, IL-13, IFN-α, TGF-β, M-CSF, IL-10, GM-CSF, IFN-γ, TNF-α, or IL-1β for 24 hours, and the results are shown in FIG. 12. A significant increase in production of VEGF was observed when stimulating THP-1 cells with rhIL-40, and its production is exacerbated when cells are co-stimulated with rhIL40 plus IL-4, IL-13, M-CSF, GM-CSF, TNF-α, or IL-1β.

### Production of Various Cytokines and Chemokines, and Growth Factors

### THP-1 Cells

THP-1 cells were stimulated with the recombinant proteins rhFc or rhIL-40 (4.63 μM)±IFN-γ (250 ng/mL), IFN-γ alone or unstimulated (control) for 24 hours. Changes in the production of 23 cytokines, chemokines, or growth factors were evaluated in the supernatant through ELISA or flow cytometry using the multiplex technology (LEGENDplex). Differences were calculated by normalizing the mean value (pg/mL) of each stimulus with the value under control conditions. An increase in the secretion of CCL3, CXCL8, CCL4, CCL2, CXCL10, CCL11, CCL5, IL-1RA, erythropoietin, PDGF-AA, and VEGF was observed when the cells were stimulated with rhIL-40. Significant differences, compared with the rest of stimuli, were found in the secretion of CCL3, CXCL8, CCL4, CCL2, CXCL10, CCL11, CCL5, CXCL9, CXCL1, CXCL11, CXCL5, CCL20, CCL17, IL-1RA, IL-6, erythropoietin, and PDGF-AA when the cells were stimulated with rhIL-40+IFN-γ. IFN-γ and rhFc+IFN-γ also had an impact on several of the evaluated molecules. The results are presented in Table 5 below.

TABLE 5
Effect of rhIL-40, in the presence or absence of IFN-γ, on the
production of chemokines and cytokines in THP-1 cells

| | | | | | rhFc + | rhIL-40 + |
|---|---|---|---|---|---|---|
| Molecule | Control | rhFc | rhIL-40 | IFN-γ | IFNγ | IFN-γ |
| CCL3 | 1 | 1 | 41 | 7 | 11 | 362 |
| CXCL8 | 1 | 1 | 9 | 2 | 1 | 78 |
| CCL4 | 1 | 1 | 10 | 6 | 6 | 485 |
| CCL2 | 1 | 2 | 6 | 39 | 41 | 1380 |
| CXCL10 | 1 | 1 | 6 | 974 | 1237 | 3746 |
| CCL11 | 1 | 2 | 5 | 4 | 3 | 31 |
| CCL5 | 1 | 1 | 4 | 1 | 2 | 25 |
| CXCL9 | 1 | 1 | 1 | 78 | 98 | 3249 |
| CXCL1 | 1 | 1 | 1 | 3 | 3 | 390 |
| CXCL11 | 1 | 2 | 1 | 2 | 3 | 26 |
| CXCL5 | 1 | 1 | 1 | 2 | 1 | 8 |
| CCL20 | 1 | 2 | 1 | 2 | 2 | 5 |
| CCL17 | 1 | 1 | 1 | 1 | 1 | 3 |
| IL-1RA | 1 | 1 | 4 | 2 | 2 | 64 |
| IL-6 | 1 | 1 | 1 | 1 | 1 | 14 |
| IL-12p70 | 1 | 1 | 1 | 1 | 1 | 1 |
| IL-12p40 | 1 | 1 | 1 | 1 | 1 | 1 |
| TNF-α | 1 | 1 | 1 | 1 | 1 | 1 |
| IL-4 | 1 | 1 | 1 | 1 | 1 | 1 |
| IL-10 | 1 | 1 | 1 | 1 | 1 | 1 |
| IL-1β | 1 | 1 | 1 | 1 | 1 | 1 |
| Arginase | 1 | 1 | 1 | 1 | 1 | 1 |
| IL-23 | 1 | 1 | 1 | 1 | 1 | 1 |
| Erythropoietin | 1 | 1 | 20 | 1 | 1 | 48 |
| PDGF-AA | 1 | 1 | 8 | 1 | 1 | 15 |
| VEGF | 1 | 1 | 2 | 2 | 2 | 2 |

### PBMCs

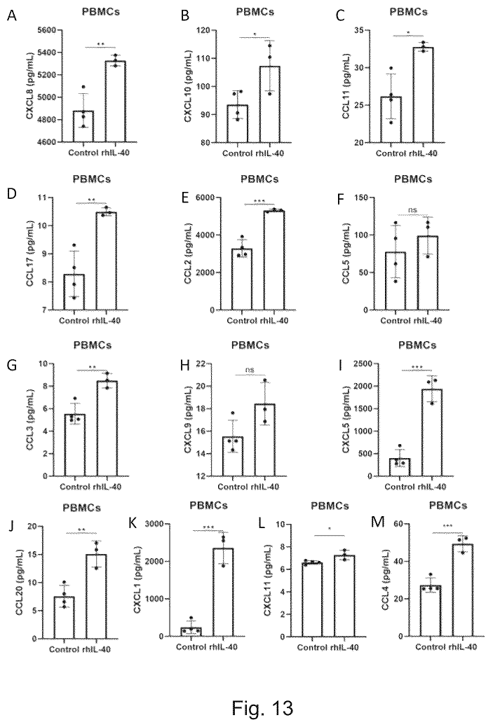

PBMCs from healthy donor's samples were isolated and stimulated with rhFc or equimolar IL-40 polypeptide concentration for 24 hours, and the results are shown in FIG. 13. Changes in the production of 13 chemokines were evaluated in the supernatant through ELISA or flow cytometry using the multiplex technology (LEGENDplex). Differences were calculated by normalizing the mean value (pg/mL) of each stimulus vs the value under control conditions. A significant increase in the secretion of CXCL1, CXCL5, CXCL8, CCL2, CCL3, CCL4, CCL17 and CCL20 was observed when the cells were stimulated with rhIL-40.

### Cell Morphology

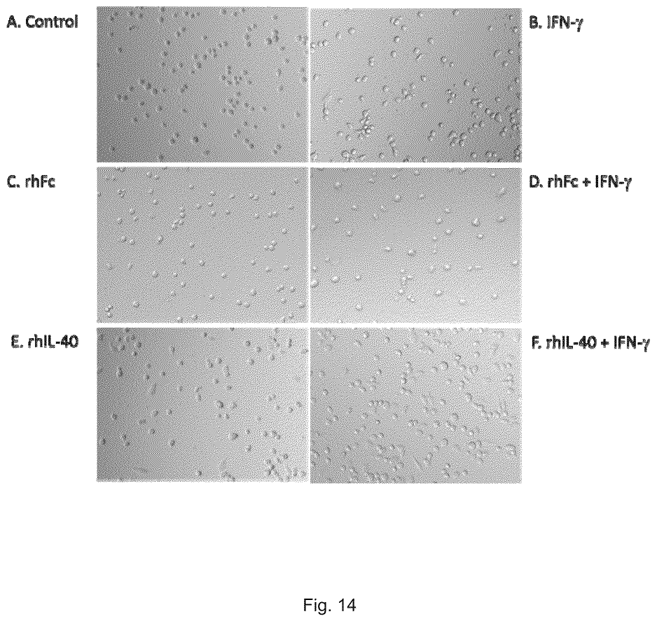

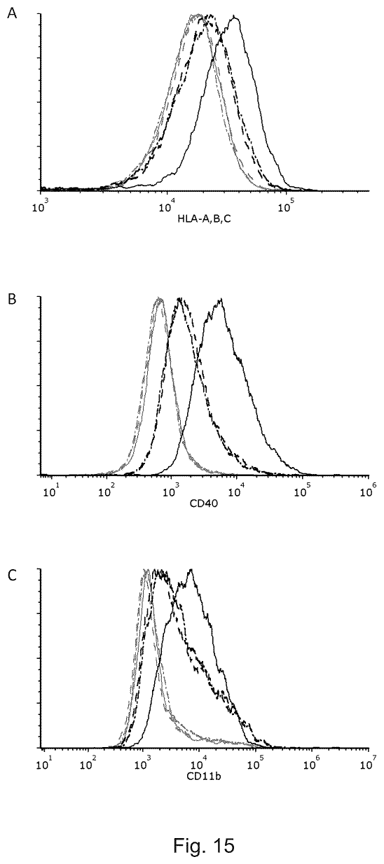

Morphological changes in THP-1 cells induced by rhIL-40, in presence or absence of IFN-γ, were observed. THP-1 cells were stimulated with IFN-γ (250 ng/mL) (FIG. 14, panel B), rhFc (2.31 uM) (FIG. 14, panel C), rhFc+IFN-γ (FIG. 14, panel D), rhIL-40 (2.31 uM) (FIG. 14, panel E), rhIL-40+IFN-γ (FIG. 14, panel F) or unstimulated (FIG. 14, panel A) for 24 hours. Morphological changes were documented through light microscopy (Keyence BZ-X700) in a phase contrast mode (magnification 10×). The percentage of cells with a morphological change was calculated counting the cells and using the following formula: (# of cells attached to the plate×100%)/total cell number. Differences in the cell shape and attachment to the plate were observed in FIG. 14, panel B (13%), FIG. 14, panel E (10%), and FIG. 14, panel F (35%). IFN-γ and IL-40 showed a synergistic effect. No changes in cell morphology were observed in FIG. 14, panel A, or FIG. 14, panel C.

Similarly, morphological changes in THP-1 cells induced by rhIL-40, in presence of IFN-γ were observed under co-stimulation with IL-10, TNF-α, and IL-1β. THP-1 cells were stimulated with each cytokine (50 ng/mL), rhFc (2.31 uM), rhFc+each cytokine, rhIL-40 (2.31 uM), rhIL-40+each cytokine or unstimulated cells for 24 hours. Morphological changes were documented through light microscopy (Keyence BZ-X700) in a phase contrast mode (magnification 10×). The number of macrophages/field is the average of 4 different fields under same conditions.

### Cell Surface Molecules

THP-1 cells were stimulated with rhFc, rhIL-40, IFN-γ, IFN-γ+rhFc or IFN-γ+rh-IL-40, and the results are shown in FIG. 15. Stimulation of rhIL40 plus a co-stimulatory cytokine as IFN-γ induces the overexpression of co-stimulatory proteins as HLA-A, B, C (Panel A), CD40 (Panel B), and CD11b (Panel C).

### Identification of IL-40 Receptor

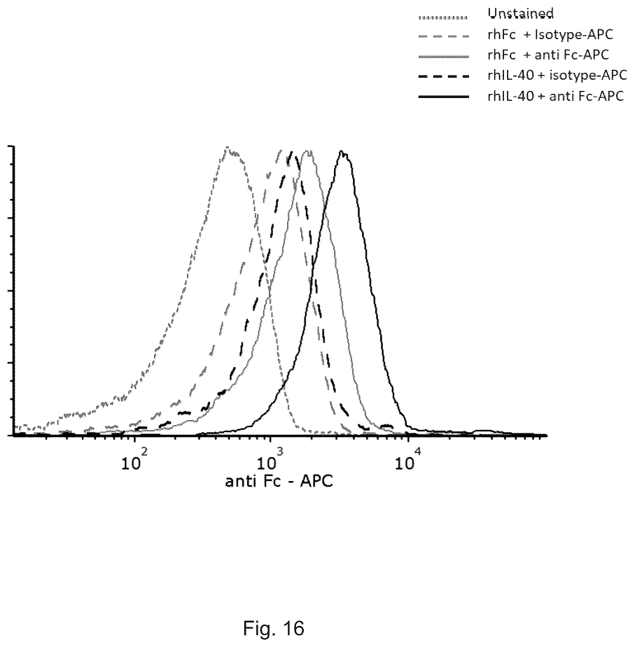

IL-40 receptor expression was observed in THP-1 cells and CD11b+ cells from a healthy donor, and the results are shown in FIG. 16. Positive cells were identified incubating cells with the rhIL-40 polypeptide coupled to Fc tag for 30 minutes at 4° C., followed by incubation with a secondary antibody anti-tag couple to APC fluorophore for 15 min at 4° C., followed by flow cytometry analysis.

### Example 2: Examples of Amino Acid and Nucleic Acid Sequences

TABLE 6
amino acid sequences and nucleic acid sequences

| | SEQ | |
|---|---|---|
| Type | ID NO | Sequence |
| Human IL-40 | 1 | MGLPGLFCLAVLAASSFSKAREEEITPVVSIAYKVLEVFPKGRWVLITCCAPQPP |
| amino acid | | PPITYSLCGTKNIKVAKKVVKTHEPASFNLNVTLKSSPDLLTYFCWASSTSGAHV |
| sequence | | DSARLQMHVVELWSKPVSELRANFTLQDRGAGPRVEMICQASSGSPPITNSLIG |
| | | KDGQVHLQQRPCHRQPANFSFLPSQTSDWFWCQAANNANVQHSALTVVPPG |
| | | GDQKMEDWQGPLESPILALPLYRSTRRLSEEEFGGFRIGNGEVRGRKAAAM |
| Human IL-40 | 2 | GCCAGGAACTAGGAGGTTCTCACTGCCCGAGCAGAGGCCCTACACCCACC |
| nucleic acid | | GAGGCATGGGGCTCCCTGGGCTGTTCTGCTTGGCCGTGCTGGCTGCCAGC |
| sequence | | AGCTTCTCCAAGGCACGGGAGGAAGAAATTACCCCTGTGGTCTCCATTGCC |
| (coding region | | TACAAAGTCCTGGAAGTTTTCCCCAAAGGCCGCTGGGTGCTCATAACCTGC |
| underlined) | | TGTGCACCCCAGCCACCACCGCCCATCACCTATTCCCTCTGTGGAACCAAG |
| | | AACATCAAGGTGGCCAAGAAGGTGGTGAAGACCCACGAGCCGGCCTCCTT |
| | | CAACCTCAACGTCACACTCAAGTCCAGTCCAGACCTGCTCACCTACTTCTGC |
| | | TGGGCGTCCTCCACCTCAGGTGCCCATGTGGACAGTGCCAGGCTACAGAT |
| | | GCACTGGGAGCTGTGGTCCAAGCCAGTGTCTGAGCTGCGGGCCAACTTCA |
| | | CTCTGCAGGACAGAGGGGCAGGCCCCAGGGTGGAGATGATCTGCCAGGC |
| | | GTCCTCGGGCAGCCCACCTATCACCAACAGCCTGATCGGGAAGGATGGGC |
| | | AGGTCCACCTGCAGCAGAGACCATGCCACAGGCAGCCTGCCAACTTCTCCT |
| | | TCCTGCCGAGCCAGACATCGGACTGGTTCTGGTGCCAGGCTGCAAACAAC |
| | | GCCAATGTCCAGCACAGCGCCCTCACAGTGGTGCCCCCAGGTGGTGACCA |
| | | GAAGATGGAGGACTGGCAGGGTCCCCTGGAGAGCCCCATCCTTGCCTTGC |
| | | CGCTCTACAGGAGCACCCGCCGTCTGAGTGAAGAGGAGTTTGGGGGGTTC |
| | | AGGATAGGGAATGGGGAGGTCAGAGGACGCAAAGCAGCAGCCATGTAGAA |
| | | TGAACCGTCCAGAGAGCCAAGCACGGCAGAGGACTGCAGGCCATCAGCGT |
| | | GCACTGTTCGTATTTGGAGTTCATGCAAAATGAGTGTGTTTTAGCTGCTCTT |
| | | GCCACAAAAAAAAAAAAAAAAAAAAAAGGGTAACTATGAGAGATGGTGGATA |
| | | TGTTAACTTGCTTCGCTATAGGAACCTTTGTGCTATCTATATTATCTATATGA |
| | | ATCCCATCATATCAGGTTGTCTACCTTA |
| Mouse IL-40 | 3 | MALLQLLLFAMLAACGFSEEQTEGITIAYKVLEVYPQSRRVLITCDAPEASQPITY |
| amino acid | | SLLASRGILVAKKVVHDSVPASFNINITIKSSPDLLTYSCQATSNSGTYGPSSRLQ |
| sequence | | MYQELWAKPVSQLQADFVLRHGDSGPTVELSCLASSGSPPITYRLVGNGGRVL |
| | | AQQRPLHGKPANFSLPLSQTTGWFQCEAENDVGVDSSARIPLPRAEARAKLVT |
| | | TLAGELPLTPTCILAGSLVSIAVIASRMLSSTGL |
| Mouse IL-40 | 4 | TAGCTGCAGGCAGCCACCCGAGGAGCATCCAGGGGCCTTGGGGTGGAGA |
| nucleic acid | | GGCCAGACAGGAAGCTTTGGACAGCCTGAGCCATACACAGCCCCAACCTC |
| sequence | | ACAGACGCATGGCGCTCCTTCAGCTGCTCCTCTTTGCCATGCTGGCTGCCT |
| (coding region | | GTGGCTTCTCAGAGGAGCAGACAGAAGGCATCACCATTGCCTACAAAGTAC |
| underlined) | | TGGAAGTTTATCCCCAAAGCCGGAGGGTGCTTATAACCTGCGATGCCCCTG |
| | | AGGCGTCCCAGCCCATCACATACTCTCTCCTGGCTAGCCGAGGTATCCTGG |
| | | TGGCAAAAAAGGTTGTGCATGACTCCGTGCCGGCCTCCTTCAACATCAATAT |
| | | CACCATCAAGTCCAGCCCAGACCTGCTCACCTACTCCTGCCAGGCAACCTC |
| | | GAACTCTGGCACCTATGGACCCAGCAGCAGGCTCCAGATGTACCAGGAACT |
| | | GTGGGCTAAGCCAGTGTCTCAGCTGCAGGCTGACTTCGTCCTACGCCATGG |
| | | GGACTCGGGCCCCACTGTGGAGCTGTCCTGCCTGGCATCCTCAGGCAGCC |
| | | CCCCCATCACCTACCGCTTGGTGGGGAATGGTGGGCGTGTTCTTGCACAG |
| | | CAAAGGCCACTTCATGGAAAACCAGCCAACTTCTCCCTCCCGCTGTCCCAG |
| | | ACCACTGGTTGGTTCCAGTGCGAAGCTGAAAACGATGTCGGTGTGGACAGC |
| | | AGTGCCCGCATCCCGCTGCCCCGAGCAGAGGCCCGAGCCAAGCTGGTGAC |
| | | CACCCTCGCAGGGGAGCTGCCCCTGACACCCACCTGTATTCTGGCTGGCA |
| | | GCCTCGTCTCCATAGCCGTTATTGCTTCCAGGATGCTGAGCTCGACCGGGT |
| | | TGTGACCGGAAGACAGAGCCATGGGCTTGCCTCCCTGCCCTGTACAAGAAC |
| | | CCACCAATGGAGCAAAAGAGAGCCTGAGGTGTGGGGGTAGAAAAGGGGGG |
| | | TTCAGGGCTGGAGAGATGGCTCAGTTACAGCACTGACTGTTCTTCCAGAGG |
| | | TCCCGAGTTCAATTCCCATAATGTACTTCTACACATAAAATAAATAATTCTTTT |
| | | TTTGTTTTGTT |
| Human IFN-γ | 5 | MKYTSYILAFQLCIVLGSLGCYCQDPYVKEAENLKKYFNAGHSDVADNGTLFLGI |
| amino acid | | LKNWKEESDRKIMQSQIVSFYFKLFKNFKDDQSIQKSVETIKEDMNVKFFNSNK |
| sequence | | KKRDDFEKLTNYSVTDLNVQRKAIHELIQVMAELSPAAKTGKRKRSQMLFRGR |
| | | RASQ |
| Human IFN-γ | 6 | ACATTGTTCTGATCATCTGAAGATCAGCTATTAGAAGAGAAAGATCAGTTAA |
| nucleic acid | | GTCCTTTGGACCTGATCAGCTTGATACAAGAACTACTGATTTCAACTTCTTTG |
| sequence | | GCTTAATTCTCTCGGAAACGATGAAATATACAAGTTATATCTTGGCTTTTCAG |
| (coding region | | CTCTGCATCGTTTTGGGTTCTCTTGGCTGTTACTGCCAGGACCCATATGTAA |
| underlined) | | AAGAAGCAGAAAACCTTAAGAAATATTTTAATGCAGGTCATTCAGATGTAGC |
| | | GGATAATGGAACTCTTTTCTTAGGCATTTTGAAGAATTGGAAAGAGGAGAGT |
| | | GACAGAAAAATAATGCAGAGCCAAATTGTCTCCTTTTACTTCAAACTTTTTAA |
| | | AAACTTTAAAGATGACCAGAGCATCCAAAAGAGTGTGGAGACCATCAAGGA |
| | | AGACATGAATGTCAAGTTTTTCAATAGCAACAAAAAGAAACGAGATGACTTC |
| | | GAAAAGCTGACTAATTATTCGGTAACTGACTTGAATGTCCAACGCAAAGCAA |
| | | TACATGAACTCATCCAAGTGATGGCTGAACTGTCGCCAGCAGCTAAAACAG |
| | | GGAAGCGAAAAAGGAGTCAGATGCTGTTTCGAGGTCGAAGAGCATCCCAGT |
| | | AATGGTTGTCCTGCCTGCAATATTTGAATTTTAAATCTAAATCTATTTATTAAT |
| | | ATTTAACATTATTTATATGGGGAATATATTTTTAGACTCATCAATCAAATAAGT |
| | | ATTTATAATAGCAACTTTTGTGTAATGAAAATGAATATCTATTAATATATGTAT |
| | | TATTTATAATTCCTATATCCTGTGACTGTCTCACTTAATCCTTTGTTTTCTGAC |
| | | TAATTAGGCAAGGCTATGTGATTACAAGGCTTTATCTCAGGGGCCAACTAGG |
| | | CAGCCAACCTAAGCAAGATCCCATGGGTTGTGTGTTTATTTCACTTGATGAT |
| | | ACAATGAACACTTATAAGTGAAGTGATACTATCCAGTTACTGCCGGTTTGAA |
| | | AATATGCCTGCAATCTGAGCCAGTGCTTTAATGGCATGTCAGACAGAACTTG |
| | | AATGTGTCAGGTGACCCTGATGAAAACATAGCATCTCAGGAGATTTCATGCC |
| | | TGGTGCTTCCAAATATTGTTGACAACTGTGACTGTACCCAAATGGAAAGTAA |
| | | CTCATTTGTTAAAATTATCAATATCTAATATATATGAATAAAGTGTAAGTTCAC |
| | | AACTA |
| Mouse IFN-γ | 7 | MNATHCILALQLFLMAVSGCYCHGTVIESLESLNNYFNSSGIDVEEKSLFLDIWR |
| amino acid | | NWQKDGDMKILQSQIISFYLRLFEVLKDNQAISNNISVIESHLITTFFSNSKAKKD |
| sequence | | AFMSIAKFEVNNPQVQRQAFNELIRVVHQLLPESSLRKRKRSRC |
| Mouse IFN-γ | 8 | TATAGCTGCCATCGGCTGACCTAGAGAAGACACATCAGCTGATCCTTTGGA |
| nucleic acid | | CCCTCTGACTTGAGACAGAAGTTCTGGGCTTCTCCTCCTGCGGCCTAGCTC |
| sequence | | TGAGACAATGAACGCTACACACTGCATCTTGGCTTTGCAGCTCTTCCTCATG |
| (coding region | | GCTGTTTCTGGCTGTTACTGCCACGGCACAGTCATTGAAAGCCTAGAAAGT |
| underlined) | | CTGAATAACTATTTTAACTCAAGTGGCATAGATGTGGAAGAAAAGAGTCTCT |
| | | TCTTGGATATCTGGAGGAACTGGCAAAAGGATGGTGACATGAAAATCCTGC |
| | | AGAGCCAGATTATCTCTTTCTACCTCAGACTCTTTGAAGTCTTGAAAGACAAT |
| | | CAGGCCATCAGCAACAACATAAGCGTCATTGAATCACACCTGATTACTACCT |
| | | TCTTCAGCAACAGCAAGGCGAAAAAGGATGCATTCATGAGTATTGCCAAGTT |
| | | TGAGGTCAACAACCCACAGGTCCAGCGCCAAGCATTCAATGAGCTCATCCG |
| | | AGTGGTCCACCAGCTGTTGCCGGAATCCAGCCTCAGGAAGCGGAAAAGGA |
| | | GTCGCTGCTGATTCGGGGTGGGGAAGAGATTGTCCCAATAAGAATAATTCT |
| | | GCCAGCACTATTTGAATTTTTAAATCTAAACCTATTTATTAATATTTAAAACTA |
| | | TTTATATGGAGAATCTATTTTAGATGCATCAACCAAAGAAGTATTTATAGTAA |
| | | CAACTTATATGTGATAAGAGTGAATTCCTATTAATATATGTGTTATTTATAATT |
| | | TCTGTCTCCTCAACTATTTCTCTTTGACCAATTAATTATTCTTTCTGACTAATT |
| | | AGCCAAGACTGTGATTGCGGGGTTGTATCTGGGGGTGGGGGACAGCCAAG |
| | | CGGCTGACTGAACTCAGATTGTAGCTTGTACCTTTACTTCACTGACCAATAA |
| | | GAAACATTCAGAGCTGCAGTGACCCCGGGAGGTGCTGCTGATGGGAGGAG |
| | | ATGTCTACACTCCGGGCCAGCGCTTTAACAGCAGGCCAGACAGCACTCGAA |
| | | TGTGTCAGGTAGTAACAGGCTGTCCCTGAAAGAAAGCAGTGTCTCAAGAGA |
| | | CTTGACACCTGGTGCTTCCCTATACAGCTGAAAACTGTGACTACACCCGAAT |
| | | GACAAATAACTCGCTCATTTATAGTTTATCACTGTCTAATTGCATATGAATA |
| | | AAGTATACCTTTGCAACCAA |
| rh IL-40-Fc | 9 | LAREEEITPVVSIAYKVLEVFPKGRWVLITCCAPQPPPPITYSLCGTKNIKVAKKV |
| amino acid | | VKTHEPASFNLNVTLKSSPDLLTYFCRASSTSGAHVDSARLQMHWELWSKPVS |
| sequence | | ELRANFTLQDRGAGPRVEMICQASSGSPPITNSLIGKDGQVHLQQRPCHRQPA |
| | | NFSFLPSQTSDWFWCQAANNANVQHSALTVVPPGGDQKMEDWQGPLESPILA |
| | | LPLYRSTRRLSEEEFGGFRIGNGEVRGRKAAAMSRIEGRMDPKSCDKTHTCPP |
| | | CPAPELLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKS |
| | | LSLSPGK |
| rh IL-40-Fc | 10 | ATGCCCCTGCTGCTGCTGCTGCCCCTGCTGTGGGCCGGCGCCCTGGCCCT |
| nucleic acid | | GGCTCGGGAGGAAGAAATTACCCCTGTGGTCTCCATTGCCTACAAAGTCCT |
| sequence | | GGAAGTTTTCCCCAAAGGCCGCTGGGTGCTCATAACCTGCTGTGCACCCCA |
| | | GCCACCACCGCCCATCACCTATTCCCTCTGTGGAACCAAGAACATCAAGGT |
| | | GGCCAAGAAGGTGGTGAAGACCCACGAGCCGGCCTCCTTCAACCTCAACG |
| | | TCACACTCAAGTCCAGTCCAGACCTGCTCACCTACTTCTGCCGGGCGTCCT |
| | | CCACCTCAGGTGCCCATGTGGACAGTGCCAGGCTACAGATGCACTGGGAG |
| | | CTGTGGTCCAAGCCAGTGTCTGAGCTGCGGGCCAACTTCACTCTGCAGGAC |
| | | AGAGGGGCAGGCCCCAGGGTGGAGATGATCTGCCAGGCGTCCTCGGGCA |
| | | GCCCACCTATCACCAACAGCCTGATCGGGAAGGATGGGCAGGTCCACCTG |
| | | CAGCAGAGACCATGCCACAGGCAGCCTGCCAACTTCTCCTTCCTGCCGAGC |
| | | CAGACATCGGACTGGTTCTGGTGCCAGGCTGCAAACAACGCCAATGTCCAG |
| | | CACAGCGCCCTCACAGTGGTGCCCCCAGGTGGTGACCAGAAGATGGAGGA |
| | | CTGGCAGGGTCCCCTGGAGAGCCCCATCCTTGCCTTGCCGCTCTACAGGA |
| | | GCACCCGCCGTCTGAGTGAAGAGGAGTTTGGGGGGTTCAGGATAGGGAAT |
| | | GGGGAGGTCAGAGGACGCAAAGCAGCAGCCATGTCTAGAATCGAGGGCCG |
| | | GATGGACCCCAAGTCCTGCGACAAGACTCACACATGCCCACCGTGCCCAG |
| | | CACCTGAACTCCTGGGGGGACCGTCAGTCTTCCTCTTCCCCCCAAAACCCA |
| | | AGGACACCCTCATGATCTCCCGGACCCCTGAGGTCACATGCGTGGTGGTG |
| | | GACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAACA |
| | | GCACGTACCGTGTGGTCAGCGTCCTCACCGTCCTGCACCAGGACTGGCTG |
| | | AATGGCAAGGAGTACAAGTGCAAGGTCTCCAACAAAGCCCTCCCAGCCCCC |
| | | ATCGAGAAAACCATCTCCAAAGCCAAAGGGCAGCCCCGAGAACCACAGGT |
| | | GTACACCCTGCCCCCATCCCGGGATGAGCTGACCAAGAACCAGGTCAGCC |
| | | TGACCTGCCTGGTCAAAGGCTTCTATCCCAGCGACATCGCCGTGGAGTGG |
| | | GAGAGCAATGGGCAGCCGGAGAACAACTACAAGACCACGCCTCCCGTGTT |
| | | GGACTCCGACGGCTCCTTCTTCCTCTACAGCAAGCTCACCGTGGACAAGAG |
| | | CAGGTGGCAGCAGGGGAACGTCTTCTCATGCTCCGTGATGCATGAGGCTCT |
| | | GCACAACCACTACACGCAGAAGAGCCTCTCCCTGTCTCCCGGGAAATAATA |
| | | A |
| rh IgG1 constant | 11 | ASTKGPSVFP LAPSSKSTSG GTAALGCLVK DYFPEPVTVS WNSGALTSGV |
| region | | HTFPAVLQSS GLYSLSSVVT VPSSSLGTQT YICNVNHKPS NTKVDKKVEP |
| | | KSCDKTHTCP PCPAPELLGG PSVFLFPPKP KDTLMISRTP EVTCVVVDVS |
| | | HEDPEVKFNW YVDGVEVHNA KTKPREEQYN STYRVVSVLT |
| | | VLHQDWLNGK EYKCKVSNKA LPAPIEKTIS KAKGQPREPQ VYTLPPSRDE |
| | | LTKNQVSLTC LVKGFYPSDI AVEWESNGQP ENNYKTTPPV LDSDGSFFLY |
| | | SKLTVDKSRW QQGNVFSCSV MHEALHNHYT QKSLSLSPGK |
| Human IL-4 | 12 | MGLTSQLLPPLFFLLACAGNFVHGHKCDITLQEIIKTLNSLTEQKTLCTELTVTDIF |
| amino acid | | AASKNTTEKETFCRAATVLRQFYSHHEKDTRCLGATAQQFHRHKQLIRFLKRLD |
| sequence | | RNLWGLAGLNSCPVKEANQSTLENFLERLKTIMREKYSKCSS |
| Human IL-4 | 13 | ATGGGTCTCACCTCCCAACTGCTTCCCCCTCTGTTCTTCCTGCTAGCATGTG |
| nucleic acid | | CCGGCAACTTTGTCCACGGACACAAGTGCGATATCACCTTACAGGAGATCA |
| sequence | | TCAAAACTTTGAACAGCCTCACAGAGCAGAAGACTCTGTGCACCGAGTTGA |
| | | CCGTAACAGACATCTTTGCTGCCTCCAAGAACACAACTGAGAAGGAAACCTT |
| | | CTGCAGGGCTGCGACTGTGCTCCGGCAGTTCTACAGCCACCATGAGAAGG |
| | | ACACTCGCTGCCTGGGTGCGACTGCACAGCAGTTCCACAGGCACAAGCAG |
| | | CTGATCCGATTCCTGAAACGGCTCGACAGGAACCTCTGGGGCCTGGCGGG |
| | | CTTGAATTCCTGTCCTGTGAAGGAAGCCAACCAGAGTACGTTGGAAAACTTC |
| | | TTGGAAAGGCTAAAGACGATCATGAGAGAGAAATATTCAAAGTGTTCGAGCT |
| | | GA |
| Mouse IL-4 | 14 | MGLNPQLVVILLFFLECTRSHIHGCDKNHLREIIGILNEVTGEGTPCTEMDVPNVL |
| amino acid | | TATKNTTESELVCRASKVLRIFYLKHGKTPCLKKNSSVLMELQRLFRAFRCLDS |
| sequence | | SISCTMNESKSTSLKDFLESLKSIMQMDYS |
| Mouse IL-4 | 15 | ATGGGTCTCAACCCCCAGCTAGTTGTCATCCTGCTCTTCTTTCTCGAATGTA |
| nucleic acid | | CCAGGAGCCATATCCACGGATGCGACAAAAATCACTTGAGAGAGATCATCG |
| sequence | | GCATTTTGAACGAGGTCACAGGAGAAGGGACGCCATGCACGGAGATGGAT |
| | | GTGCCAAACGTCCTCACAGCAACGAAGAACACCACAGAGAGTGAGCTCGTC |
| | | TGTAGGGCTTCCAAGGTGCTTCGCATATTTTATTTAAAACATGGGAAAACTC |
| | | CATGCTTGAAGAAGAACTCTAGTGTTCTCATGGAGCTGCAGAGACTCTTTCG |
| | | GGCTTTTCGATGCCTGGATTCATCGATAAGCTGCACCATGAATGAGTCCAA |
| | | GTCCACATCACTGAAAGACTTCCTGGAAAGCCTAAAGAGCATCATGCAAATG |
| | | GATTACTCGTAG |
| Human IL-10 | 16 | MHSSALLCCLVLLTGVRASPGQGTQSENSCTHFPGNLPNMLRDLRDAFSRVKT |
| amino acid | | FFQMKDQLDNLLLKESLLEDFKGYLGCQALSEMIQFYLEEVMPQAENQDPDIKA |
| sequence | | HVNSLGENLKTLRLRLRRCHRFLPCENKSKAVEQVKNAFNKLQEKGIYKAMSE |
| | | FDIFINYIEAYMTMKIRN |
| Human IL-10 | 17 | ATGCACAGCTCAGCACTGCTCTGTTGCCTGGTCCTCCTGACTGGGGTGAGG |
| nucleic acid | | GCCAGCCCAGGCCAGGGCACCCAGTCTGAGAACAGCTGCACCCACTTCCC |
| sequence | | AGGCAACCTGCCTAACATGCTTCGAGATCTCCGAGATGCCTTCAGCAGAGT |
| | | GAAGACTTTCTTTCAAATGAAGGATCAGCTGGACAACTTGTTGTTAAAGGAG |
| | | TCCTTGCTGGAGGACTTTAAGGGTTACCTGGGTTGCCAAGCCTTGTCTGAG |
| | | ATGATCCAGTTTTACCTGGAGGAGGTGATGCCCCAAGCTGAGAACCAAGAC |
| | | CCAGACATCAAGGCGCATGTGAACTCCCTGGGGGAGAACCTGAAGACCCT |
| | | CAGGCTGAGGCTACGGCGCTGTCATCGATTTCTTCCCTGTGAAAACAAGAG |
| | | CAAGGCCGTGGAGCAGGTGAAGAATGCCTTTAATAAGCTCCAAGAGAAAGG |
| | | CATCTACAAAGCCATGAGTGAGTTTGACATCTTCATCAACTACATAGAAGCC |
| | | TACATGACAATGAAGATACGAAACTGA |
| Mouse IL-10 | 18 | MPGSALLCCLLLLTGMRISRGQYSREDNNCTHFPVGQSHMLLELRTAFSQVKT |
| amino acid | | FFQTKDQLDNILLTDSLMQDFKGYLGCQALSEMIQFYLVEVMPQAEKHGPEIKE |
| sequence | | HLNSLGEKLKTLRMRLRRCHRFLPCENKSKAVEQVKSDFNKLQDQGVYKAMN |
| | | EFDIFINCIEAYMMIKMKS |
| Mouse IL-10 | 19 | ATGCCTGGCTCAGCACTGCTATGCTGCCTGCTCTTACTGACTGGCATGAGG |
| nucleic acid | | ATCAGCAGGGGCCAGTACAGCCGGGAAGACAATAACTGCACCCACTTCCCA |
| sequence | | GTCGGCCAGAGCCACATGCTCCTAGAGCTGCGGACTGCCTTCAGCCAGGT |
| | | GAAGACTTTCTTTCAAACAAAGGACCAGCTGGACAACATACTGCTAACCGAC |
| | | TCCTTAATGCAGGACTTTAAGGGTTACTTGGGTTGCCAAGCCTTATCGGAAA |
| | | TGATCCAGTTTTACCTGGTAGAAGTGATGCCCCAGGCAGAGAAGCATGGCC |
| | | CAGAAATCAAGGAGCATTTGAATTCCCTGGGTGAGAAGCTGAAGACCCTCA |
| | | GGATGCGGCTGAGGCGCTGTCATCGATTTCTCCCCTGTGAAAATAAGAGCA |
| | | AGGCAGTGGAGCAGGTGAAGAGTGATTTTAATAAGCTCCAAGACCAAGGTG |
| | | TCTACAAGGCCATGAATGAATTTGACATCTTCATCAACTGCATAGAAGCATA |
| | | CATGATGATCAAAATGAAAAGCTAA |
| Human TGF-β | 20 | MPPSGLRLLPLLLPLLWLLVLTPGRPAAGLSTCKTIDMELVKRKRIEAIRGQILSK |
| amino acid | | LRLASPPSQGEVPPGPLPEAVLALYNSTRDRVAGESAEPEPEPEADYYAKEVT |
| sequence | | RVLMVETHNEIYDKFKQSTHSIYMFFNTSELREAVPEPVLLSRAELRLLRLKLKV |
| | | EQHVELYQKYSNNSWRYLSNRLLAPSDSPEWLSFDVTGVVRQWLSRGGEIEG |
| | | FRLSAHCSCDSRDNTLQVDINGFTTGRRGDLATIHGMNRPFLLLMATPLERAQ |
| | | HLQSSRHRRALDTNYCFSSTEKNCCVRQLYIDFRKDLGVVKWIHEPKGYHANFC |
| | | LGPCPYIWSLDTQYSKVLALYNQHNPGASAAPCCVPQALEPLPIVYYVGRKPKV |
| | | EQLSNMIVRSCKCS |
| Human TGF-β | 21 | ATGCCGCCCTCCGGGCTGCGGCTGCTGCCGCTGCTGCTACCGCTGCTGTG |
| nucleic acid | | GCTACTGGTGCTGACGCCTGGCCGGCCGGCCGCGGGACTATCCACCTGCA |
| sequence | | AGACTATCGACATGGAGCTGGTGAAGCGGAAGCGCATCGAGGCCATCCGC |
| | | GGCCAGATCCTGTCCAAGCTGCGGCTCGCCAGCCCCCCGAGCCAGGGGG |
| | | AGGTGCCGCCCGGCCCGCTGCCCGAGGCCGTGCTCGCCCTGTACAACAG |
| | | CACCCGCGACCGGGTGGCCGGGGAGAGTGCAGAACCGGAGCCCGAGCCT |
| | | GAGGCCGACTACTACGCCAAGGAGGTCACCCGCGTGCTAATGGTGGAAAC |
| | | CCACAACGAAATCTATGACAAGTTCAAGCAGAGTACACACAGCATATATATG |
| | | TTCTTCAACACATCAGAGCTCCGAGAAGCGGTACCTGAACCCGTGTTGCTC |
| | | TCCCGGGCAGAGCTGCGTCTGCTGAGGCTCAAGTTAAAAGTGGAGCAGCA |
| | | CGTGGAGCTGTACCAGAAATACAGCAACAATTCCTGGCGATACCTCAGCAA |
| | | CCGGCTGCTGGCACCCAGCGACTCGCCAGAGTGGTTATCTTTTGATGTCAC |
| | | CGGAGTTGTGCGGCAGTGGTTGAGCCGTGGAGGGGAAATTGAGGGCTTTC |
| | | GCCTTAGCGCCCACTGCTCCTGTGACAGCAGGGATAACACACTGCAAGTGG |
| | | ACATCAACGGGTTCACTACCGGCCGCCGAGGTGACCTGGCCACCATTCATG |
| | | GCATGAACCGGCCTTTCCTGCTTCTCATGGCCACCCCGCTGGAGAGGGCC |
| | | CAGCATCTGCAAAGCTCCCGGCACCGCCGAGCCCTGGACACCAACTATTG |
| | | CTTCAGCTCCACGGAGAAGAACTGCTGCGTGCGGCAGCTGTACATTGACTT |
| | | CCGCAAGGACCTCGGCTGGAAGTGGATCCACGAGCCCAAGGGCTACCATG |
| | | CCAACTTCTGCCTCGGGCCCTGCCCCTACATTTGGAGCCTGGACACGCAGT |
| | | ACAGCAAGGTCCTGGCCCTGTACAACCAGCATAACCCGGGCGCCTCGGCG |
| | | GCGCCGTGCTGCGTGCCGCAGGCGCTGGAGCCGCTGCCCATCGTGTACTA |
| | | CGTGGGCCGCAAGCCCAAGGTGGAGCAGCTGTCCAACATGATCGTGCGCT |
| | | CCTGCAAGTGCAGCTGA |
| Mouse TGF-β | 22 | MPPSGLRLLPLLLPLPWLLVLTPGRPAAGLSTCKTIDMELVKRKRIEAIRGQILSK |
| amino acid | | LRLASPPSQGEVPPGPLPEAVLALYNSTRDRVAGESADPEPEPEADYYAKEVT |
| sequence | | RVLMVDRNNAIYEKTKDISHSIYMFFNTSDIREAVPEPPLLSRAELRLQRLKSSV |
| | | EQHVELYQKYSNNSWRYLGNRLLTPTDTPEWLSFDVTGVVRQWLNQGDGIQG |
| | | FRFSAHCSCDSKDNKLHVEINGISPKRRGDLGTIHDMNRPFLLLMATPLERAQH |
| | | LHSSRHRRALDTNYCFSSTEKNCCVRQLYIDFRKDLGWKWIHEPKGYHANFCL |
| | | GPCPYIWSLDTQYSKVLALYNQHNPGASASPCCVPQALEPLPIVYYVGRKPKV |
| | | EQLSNMIVRSCKCS |
| Mouse TGF-β | 23 | ATGCCGCCCTCGGGGCTGCGGCTACTGCCGCTTCTGCTCCCACTCCCGTG |
| nucleic acid | | GCTTCTAGTGCTGACGCCCGGGAGGCCAGCCGCGGGACTCTCCACCTGCA |
| sequence | | AGACCATCGACATGGAGCTGGTGAAACGGAAGCGCATCGAAGCCATCCGT |
| | | GGCCAGATCCTGTCCAAACTAAGGCTCGCCAGTCCCCCAAGCCAGGGGGA |
| | | GGTACCGCCCGGCCCGCTGCCCGAGGCGGTGCTCGCTTTGTACAACAGCA |
| | | CCCGCGACCGGGTGGCAGGCGAGAGCGCCGACCCAGAGCCGGAGCCCGA |
| | | AGCGGACTACTATGCTAAAGAGGTCACCCGCGTGCTAATGGTGGACCGCAA |
| | | CAACGCCATCTATGAGAAAACCAAAGACATCTCACACAGTATATATATGTTC |
| | | TTCAATACGTCAGACATTCGGGAAGCAGTGCCCGAACCCCCATTGCTGTCC |
| | | CGTGCAGAGCTGCGCTTGCAGAGATTAAAATCAAGTGTGGAGCAACATGTG |
| | | GAACTCTACCAGAAATATAGCAACAATTCCTGGCGTTACCTTGGTAACCGGC |
| | | TGCTGACCCCCACTGATACGCCTGAGTGGCTGTCTTTTGACGTCACTGGAG |
| | | TTGTACGGCAGTGGCTGAACCAAGGAGACGGAATACAGGGCTTTCGATTCA |
| | | GCGCTCACTGCTCTTGTGACAGCAAAGATAACAAACTCCACGTGGAAATCA |
| | | ACGGGATCAGCCCCAAACGTCGGGGCGACCTGGGCACCATCCATGACATG |
| | | AACCGGCCCTTCCTGCTCCTCATGGCCACCCCCCTGGAAAGGGCCCAGCA |
| | | CCTGCACAGCTCACGGCACCGGAGAGCCCTGGATACCAACTATTGCTTCAG |
| | | CTCCACAGAGAAGAACTGCTGTGTGCGGCAGCTGTACATTGACTTTAGGAA |
| | | GGACCTGGGTTGGAAGTGGATCCACGAGCCCAAGGGCTACCATGCCAACT |
| | | TCTGTCTGGGACCCTGCCCCTATATTTGGAGCCTGGACACACAGTACAGCA |
| | | AGGTCCTTGCCCTCTACAACCAACACAACCCGGGCGCTTCGGCGTCACCGT |
| | | GCTGCGTGCCGCAGGCTTTGGAGCCACTGCCCATCGTCTACTACGTGGGT |
| | | CGCAAGCCCAAGGTGGAGCAGTTGTCCAACATGATTGTGCGCTCCTGCAAG |
| | | TGCAGCTGA |
| Human GM-CSF | 24 | MWLQSLLLLGTVACSISAPARSPSPSTQPWEHVNAIQEARRLLNLSRDTAAEM |
| amino acid | | NETVEVISEMFDLQEPTCLQTRLELYKQGLRGSLTKLKGPLTMMASHYKQHCP |
| sequence | | PTPETSCATQIITFESFKENLKDFLLVIPFDCWEPVQE |
| Human GM-CSF | 25 | AGTACACAGAGAGAAAGGCTAAAGTTCTCTGGAGGATGTGGCTGCAGAGCC |
| nucleic acid | | TGCTGCTCTTGGGCACTGTGGCCTGCAGCATCTCTGCACCCGCCCGCTCG |
| sequence | | CCCAGCCCCAGCACGCAGCCCTGGGAGCATGTGAATGCCATCCAGGAGGC |
| | | CCGGCGTCTCCTGAACCTGAGTAGAGACACTGCTGCTGAGATGAATGAAAC |
| | | AGTAGAAGTCATCTCAGAAATGTTTGACCTCCAGGAGCCGACCTGCCTACA |
| | | GACCCGCCTGGAGCTGTACAAGCAGGGCCTGCGGGGCAGCCTCACCAAGC |
| | | TCAAGGGCCCCTTGACCATGATGGCCAGCCACTACAAGCAGCACTGCCCTC |
| | | CAACCCCGGAAACTTCCTGTGCAACCCAGATTATCACCTTTGAAAGTTTCAA |
| | | AGAGAACCTGAAGGACTTTCTGCTTGTCATCCCCTTTGACTGCTGGGAGCC |
| | | AGTCCAGGAGTGAGACCGGCCAGATGAGGCTGGCCAAGCCGGGGAGCTG |
| | | CTCTCTCATGAAACAAGAGCTAGAAACTCAGGATGGTCATCTTGGAGGGAC |
| | | CAAGGGGTGGGCCACAGCCATGGTGGGAGTGGCCTGGACCTGCCCTGGG |
| | | CCACACTGACCCTGATACAGGCATGGCAGAAGAATGGGAATATTTTATACTG |
| | | ACAGAAATCAGTAATATTTATATATTTATATTTTTAAAATATTTATTTATTTATT |
| | | TATTTAAGTTCATATTCCATATTTATTCAAGATGTTTTACCGTAATAATTATTAT |
| | | TAAAAATATGCTTCTACTTG |
| Mouse GM-CSF | 26 | MWLQNLLFLGIVVYSLSAPTRSPITVTRPWKHVEAIKEALNLLDDMPVTLNEEVE |
| amino acid | | VVSNEFSFKKLTCVQTRLKIFEQGLRGNFTKLKGALNMTASYYQTYCPPTPETD |
| sequence | | CETQVTTYADFIDSLKTFLTDIPFECKKPGQK |
| Mouse GM-CSF | 27 | ATGTGGCTGCAGAATTTACTTTTCCTGGGCATTGTGGTCTACAGCCTCTCAG |
| nucleic acid | | CACCCACCCGCTCACCCATCACTGTCACCCGGCCTTGGAAGCATGTAGAGG |
| sequence | | CCATCAAAGAAGCCCTGAACCTCCTGGATGACATGCCTGTCACGTTGAATG |
| | | AAGAGGTAGAAGTCGTCTCTAACGAGTTCTCCTTCAAGAAGCTAACATGTGT |
| | | GCAGACCCGCCTGAAGATATTCGAGCAGGGTCTACGGGGCAATTTCACCAA |
| | | ACTCAAGGGCGCCTTGAACATGACAGCCAGCTACTACCAGACATACTGCCC |
| | | CCCAACTCCGGAAACGGACTGTGAAACACAAGTTACCACCTATGCGGATTT |
| | | CATAGACAGCCTTAAAACCTTTCTGACTGATATCCCCTTTGAATGCAAAAAA |
| | | CCAGGCCAAAAATGA |
| Human M-CSF | 28 | MTAPGAAGRCPPTTWLGSLLLLVCLLASRSITEEVSEYCSHMIGSGHLQSLQRL |
| amino acid | | IDSQMETSCQITFEFVDQEQLKDPVCYLKKAFLLVQDIMEDTMRFRDNTPNAIAI |
| sequence | | VQLQELSLRLKSCFTKDYEEHDKACVRTFYETPLQLLEKVKNVFNETKNLLDKD |
| | | WNIFSKNCNNSFAECSSQDVVTKPDCNCLYPKAIPSSDPASVSPHQPLAPSMA |
| | | PVAGLTWEDSEGTEGSSLLPGEQPLHTVDPGSAKQRPPRSTCQSFEPPETPV |
| | | VKDSTIGGSPQPRPSVGAFNPGMEDILDSAMGTNWVPEEASGEASEIPVPQGT |
| | | ELSPSRPGGGSMQTEPARPSNFLSASSPLPASAKGQQPADVTGTALPRVGPV |
| | | RPTGQDWNHTPQKTDHPSALLRDPPEPGSPRISSLRPQGLSNPSTLSAQPQLS |
| | | RSHSSGSVLPLGELEGRRSTRDRRSPAEPEGGPASEGAARPLPRFNSVPLTDT |
| | | GHERQSEGSFSPQLQESVFHLLVPSVILVLLAVGGLLFYRWRRRSHQEPQRAD |
| | | SPLEQPEGSPLTQDDRQVELPV |
| Human M-CSF | 29 | ATGACCGCGCCGGGCGCCGCCGGGCGCTGCCCTCCCACGACATGGCTGG |
| nucleic acid | | GCTCCCTGCTGTTGTTGGTCTGTCTCCTGGCGAGCAGGAGTATCACCGAGG |
| sequence | | AGGTGTCGGAGTACTGTAGCCACATGATTGGGAGTGGACACCTGCAGTCTC |
| | | TGCAGCGGCTGATTGACAGTCAGATGGAGACCTCGTGCCAAATTACATTTG |
| | | AGTTTGTAGACCAGGAACAGTTGAAAGATCCAGTGTGCTACCTTAAGAAGG |
| | | CATTTCTCCTGGTACAAGACATAATGGAGGACACCATGCGCTTCAGAGATAA |
| | | CACCCCCAATGCCATCGCCATTGTGCAGCTGCAGGAACTCTCTTTGAGGCT |
| | | GAAGAGCTGCTTCACCAAGGATTATGAAGAGCATGACAAGGCCTGCGTCCG |
| | | AACTTTCTATGAGACACCTCTCCAGTTGCTGGAGAAGGTCAAGAATGTCTTT |
| | | AATGAAACAAAGAATCTCCTTGACAAGGACTGGAATATTTTCAGCAAGAACT |
| | | GCAACAACAGCTTTGCTGAATGCTCCAGCCAAGATGTGGTGACCAAGCCTG |
| | | ATTGCAACTGCCTGTACCCCAAAGCCATCCCTAGCAGTGACCCGGCCTCTG |
| | | TCTCCCCTCATCAGCCCCTCGCCCCCTCCATGGCCCCTGTGGCTGGCTTGA |
| | | CCTGGGAGGACTCTGAGGGAACTGAGGGCAGCTCCCTCTTGCCTGGTGAG |
| | | CAGCCCCTGCACACAGTGGATCCAGGCAGTGCCAAGCAGCGGCCACCCAG |
| | | GAGCACCTGCCAGAGCTTTGAGCCGCCAGAGACCCCAGTTGTCAAGGACA |
| | | GCACCATCGGTGGCTCACCACAGCCTCGCCCCTCTGTCGGGGCCTTCAAC |
| | | CCCGGGATGGAGGATATTCTTGACTCTGCAATGGGCACTAATTGGGTCCCA |
| | | GAAGAAGCCTCTGGAGAGGCCAGTGAGATTCCCGTACCCCAAGGGACAGA |
| | | GCTTTCCCCCTCCAGGCCAGGAGGGGGCAGCATGCAGACAGAGCCCGCCA |
| | | GACCCAGCAACTTCCTCTCAGCATCTTCTCCACTCCCTGCATCAGCAAAGG |
| | | GCCAACAGCCGGCAGATGTAACTGGTACCGCCTTGCCCAGGGTGGGCCCC |
| | | GTGAGGCCCACTGGCCAGGACTGGAATCACACCCCCCAGAAGACAGACCA |
| | | TCCATCTGCCCTGCTCAGAGACCCCCCGGAGCCAGGCTCTCCCAGGATCT |
| | | CATCACTGCGCCCCCAGGGCCTCAGCAACCCCTCCACCCTCTCTGCTCAGC |
| | | CACAGCTTTCCAGAAGCCACTCCTCGGGCAGCGTGCTGCCCCTTGGGGAG |
| | | CTGGAGGGCAGGAGGAGCACCAGGGATCGGAGGAGCCCCGCAGAGCCAG |
| | | AAGGAGGACCAGCAAGTGAAGGGGCAGCCAGGCCCCTGCCCCGTTTTAAC |
| | | TCCGTTCCTTTGACTGACACAGGCCATGAGAGGCAGTCCGAGGGATCCTTC |
| | | AGCCCGCAGCTCCAGGAGTCTGTCTTCCACCTGCTGGTGCCCAGTGTCATC |
| | | CTGGTCTTGCTGGCCGTCGGAGGCCTCTTGTTCTACAGGTGGAGGCGGCG |
| | | GAGCCATCAAGAGCCTCAGAGAGCGGATTCTCCCTTGGAGCAACCAGAGG |
| | | GCAGCCCCCTGACTCAGGATGACAGACAGGTGGAACTGCCAGTGTAG |
| Mouse M-CSF | 30 | MTARGAAGRCPSSTWLGSRLLLVCLLMSRSIAKEVSEHCSHMIGNGHLKVLQQ |
| amino acid | | LIDSQMETSCQIAFEFVDQEQLDDPVCYLKKAFFLVQDIIDETMRFKDNTPNANA |
| sequence | | TERLQELSNNLNSCFTKDYEEQNKACVRTFHETPLQLLEKIKNFFNETKNLLEK |
| | | DWNIFTKNCNNSFAKCSSRDVVTKPDCNCLYPKATPSSDPASASPHQPPAPSM |
| | | APLAGLAWDDSQRTEGSSLLPSELPLRIEDPGSAKQRPPRSTCQTLESTEQPN |
| | | HGDRLTEDSQPHPSAGGPVPGVEDILESSLGTNWVLEEASGEASEGFLTQEAK |
| | | FSPSTPVGGSIQAETDRPRALSASPFPKSTEDQKPVDITDRPLTEVNPMRPIGQ |
| | | TQNNTPEKTDGTSTLREDHQEPGSPHIATPNPQRVSNSATPVAQLLLPKSHSW |
| | | GIVLPLGELEGKRSTRDRRSPAELEGGSASEGAARPVARFNSIPLTDTGHVEQH |
| | | EGSSDPQIPESVFHLLVPGIILVLLTVGGLLFYKWKWRSHRDPQTLDSSVGRPE |
| | | DSSLTQDEDRQVELPV |
| Mouse M-CSF | 31 | ATGACCGCGCGGGGCGCCGCGGGGCGCTGCCCTTCTTCGACATGGCTGG |
| nucleic acid | | GCTCCCGGCTGCTGCTGGTCTGTCTCCTCATGAGCAGGAGTATTGCCAAGG |
| sequence | | AGGTGTCAGAACACTGTAGCCACATGATTGGGAATGGACACCTGAAGGTCC |
| | | TGCAGCAGTTGATCGACAGTCAAATGGAGACTTCATGCCAGATTGCCTTTGA |
| | | ATTTGTAGACCAGGAACAGCTGGATGATCCTGTTTGCTACCTAAAGAAGGC |
| | | CTTTTTTCTGGTACAAGACATAATAGATGAGACCATGCGCTTTAAAGACAAC |
| | | ACCCCCAATGCTAACGCCACCGAGAGGCTCCAGGAACTCTCCAATAACCTG |
| | | AACAGCTGCTTCACCAAGGACTATGAGGAGCAGAACAAGGCCTGTGTCCGA |
| | | ACTTTCCATGAGACTCCTCTCCAGCTGCTGGAGAAGATCAAGAACTTCTTTA |
| | | ATGAAACAAAGAATCTCCTTGAAAAGGACTGGAACATTTTTACCAAGAACTG |
| | | CAACAACAGCTTTGCTAAGTGCTCTAGCCGAGATGTGGTGACCAAGCCTGA |
| | | TTGCAACTGCCTGTACCCTAAAGCCACCCCTAGCAGTGACCCGGCCTCTGC |
| | | CTCCCCTCACCAGCCCCCCGCCCCCTCCATGGCCCCTCTGGCTGGCTTGG |
| | | CTTGGGATGATTCTCAGAGGACAGAGGGCAGCTCCCTCTTGCCCAGTGAGC |
| | | TTCCCCTTCGCATAGAGGACCCAGGCAGTGCCAAGCAGCGACCACCCAGG |
| | | AGTACCTGCCAGACCCTCGAGTCAACAGAGCAACCAAACCATGGGGACAGA |
| | | CTCACTGAGGACTCACAACCTCATCCTTCTGCGGGGGGGCCCGTCCCTGG |
| | | GGTGGAAGACATTCTTGAATCTTCACTGGGCACTAACTGGGTCCTAGAAGA |
| | | AGCTTCTGGAGAGGCTAGTGAGGGATTTTTGACCCAGGAAGCAAAGTTTTC |
| | | CCCCTCCACGCCTGTAGGGGGCAGCATCCAGGCAGAGACTGACAGACCCA |
| | | GGGCCCTCTCAGCATCTCCATTCCCTAAATCAACAGAGGACCAAAAGCCAG |
| | | TGGATATAACAGACAGGCCGTTGACAGAGGTGAACCCTATGAGACCCATTG |
| | | GCCAGACACAGAATAATACTCCTGAGAAGACTGATGGTACATCCACGCTGC |
| | | GTGAAGACCACCAGGAGCCAGGCTCTCCCCATATTGCGACACCGAATCCCC |
| | | AACGAGTCAGCAACTCAGCCACCCCCGTTGCTCAGTTACTGCTTCCCAAAA |
| | | GCCACTCTTGGGGCATTGTGCTGCCCCTTGGGGAGCTTGAGGGCAAGAGA |
| | | AGTACCAGGGATCGAAGGAGCCCCGCAGAGCTGGAAGGAGGATCAGCAAG |
| | | TGAGGGGGCAGCCAGGCCTGTGGCCCGTTTTAATTCCATTCCTTTGACTGA |
| | | CACAGGCCATGTGGAGCAGCATGAGGGATCCTCTGACCCCCAGATCCCTG |
| | | AGTCTGTCTTCCACCTGCTGGTGCCGGGCATCATCCTAGTCTTGCTGACTG |
| | | TTGGGGGCCTCCTGTTCTACAAGTGGAAGTGGAGGAGCCATCGAGACCCT |
| | | CAGACATTGGATTCTTCTGTGGGGCGACCAGAGGACAGCTCCCTGACCCAG |
| | | GATGAGGACAGACAGGTGGAACTGCCAGTATAG |
| Human IFN-α | 32 | MALTFALLVALLVLSCKSSCSVGCDLPQTHSLGSRRTLMLLAQMRRISLFSCLK |
| amino acid | | DRHDFGFPQEEFGNQFQKAETIPVLHEMIQQIFNLFSTKDSSAAWDETLLDKFY |
| sequence | | TELYQQLNDLEACVIQGVGVTETPLMKEDSILAVRKYFQRITLYLKEKKYSPCAW |
| | | EVVRAEIMRSFSLSTNLQESLRSKE |
| Human IFN-α | 33 | ATGGCCTTGACCTTTGCTTTACTGGTGGCCCTCCTGGTGCTCAGCTGCAAG |
| nucleic acid | | TCAAGCTGCTCTGTGGGCTGTGATCTGCCTCAAACCCACAGCCTGGGTAGC |
| sequence | | AGGAGGACCTTGATGCTCCTGGCACAGATGAGGAGAATCTCTCTTTTCTCC |
| | | TGCTTGAAGGACAGACATGACTTTGGATTTCCCCAGGAGGAGTTTGGCAAC |
| | | CAGTTCCAAAAGGCTGAAACCATCCCTGTCCTCCATGAGATGATCCAGCAG |
| | | ATCTTCAATCTCTTCAGCACAAAGGACTCATCTGCTGCTTGGGATGAGACCC |
| | | TCCTAGACAAATTCTACACTGAACTCTACCAGCAGCTGAATGACCTGGAAGC |
| | | CTGTGTGATACAGGGGGTGGGGGTGACAGAGACTCCCCTGATGAAGGAGG |
| | | ACTCCATTCTGGCTGTGAGGAAATACTTCCAAAGAATCACTCTCTATCTGAA |
| | | AGAGAAGAAATACAGCCCTTGTGCCTGGGAGGTTGTCAGAGCAGAAATCAT |
| | | GAGATCTTTTTCTTTGTCAACAAACTTGCAAGAAAGTTTAAGAAGTAAGGAAT |
| | | GA |
| Mouse IFN-α | 34 | MARLCAFLMTLLVMSYWSTCSLGCDLPQTHNLRNKRALTLLVQMRRLSPLSCL |
| amino acid | | KDRKDFRFPQEKVDAQQIQNAQAIPVLQELTQQVLNIFTSKDSSAAWDASLLDS |
| sequence | | FCNDLHQQLNDLKACVMQEVGVQEPPLTQEDYLLAVRTYFHRITVYLREKKHS |
| | | PCAWEVVRAEVWRAMSSSAKLLARLSEEKE |
| Mouse IFN-α | 35 | ATGGCTAGGCTCTGTGCTTTCCTGATGACCCTGCTAGTGATGAGCTACTGG |
| nucleic acid | | TCAACCTGCTCTCTAGGATGTGACCTGCCTCAGACTCATAACCTCAGGAACA |
| sequence | | AGAGAGCCTTGACCCTCCTGGTACAAATGAGGAGACTCTCCCCTCTCTCCT |
| | | GCCTGAAGGACAGAAAGGACTTTAGATTCCCCCAGGAGAAGGTGGATGCC |
| | | CAGCAGATCCAGAATGCTCAAGCCATCCCTGTCCTACAAGAGCTGACCCAG |
| | | CAGGTCCTGAACATCTTCACATCAAAGGACTCATCTGCTGCTTGGGATGCAT |
| | | CCCTCCTAGACTCATTCTGCAATGACCTCCATCAGCAGCTCAATGACCTCAA |
| | | AGCCTGTGTGATGCAGGAGGTGGGGGTGCAGGAACCTCCCCTGACCCAGG |
| | | AAGACTACCTGCTGGCTGTGAGGACATACTTCCACAGGATCACTGTGTACC |
| | | TGAGAGAGAAGAAACACAGCCCCTGTGCCTGGGAGGTGGTCAGAGCAGAA |
| | | GTCTGGAGAGCCATGTCTTCCTCAGCCAAGTTGCTGGCAAGACTGAGTGAG |
| | | GAGAAGGAGTGA |
| Human IL-1β | 36 | MAEVPELASEMMAYYSGNEDDLFFEADGPKQMKCSFQDLDLCPLDGGIQLRIS |
| amino acid | | DHHYSKGFRQAASVVVAMDKLRKMLVPCPQTFQENDLSTFFPFIFEEEPIFFDT |
| sequence | | WDNEAYVHDAPVRSLNCTLRDSQQKSLVMSGPYELKALHLQGQDMEQQVVF |
| | | SMSFVQGEESNDKIPVALGLKEKNLYLSCVLKDDKPTLQLESVDPKNYPKKKME |
| | | KRFVFNKIEINNKLEFESAQFPNWYISTSQAENMPVFLGGTKGGQDITDFTMQF |
| | | VSS |
| Human IL-1β | 37 | ATGGCAGAAGTACCTGAGCTCGCCAGTGAAATGATGGCTTATTACAGTGGC |
| nucleic acid | | AATGAGGATGACTTGTTCTTTGAAGCTGATGGCCCTAAACAGATGAAGTGCT |
| sequence | | CCTTCCAGGACCTGGACCTCTGCCCTCTGGATGGCGGCATCCAGCTACGAA |
| | | TCTCCGACCACCACTACAGCAAGGGCTTCAGGCAGGCCGCGTCAGTTGTTG |
| | | TGGCCATGGACAAGCTGAGGAAGATGCTGGTTCCCTGCCCACAGACCTTCC |
| | | AGGAGAATGACCTGAGCACCTTCTTTCCCTTCATCTTTGAAGAAGAACCTAT |
| | | CTTCTTCGACACATGGGATAACGAGGCTTATGTGCACGATGCACCTGTACG |
| | | ATCACTGAACTGCACGCTCCGGGACTCACAGCAAAAAAGCTTGGTGATGTC |
| | | TGGTCCATATGAACTGAAAGCTCTCCACCTCCAGGGACAGGATATGGAGCA |
| | | ACAAGTGGTGTTCTCCATGTCCTTTGTACAAGGAGAAGAAAGTAATGACAAA |
| | | ATACCTGTGGCCTTGGGCCTCAAGGAAAAGAATCTGTACCTGTCCTGCGTG |
| | | TTGAAAGATGATAAGCCCACTCTACAGCTGGAGAGTGTAGATCCCAAAAATT |
| | | ACCCAAAGAAGAAGATGGAAAAGCGATTTGTCTTCAACAAGATAGAAATCAA |
| | | TAACAAGCTGGAATTTGAGTCTGCCCAGTTCCCCAACTGGTACATCAGCAC |
| | | CTCTCAAGCAGAAAACATGCCCGTCTTCCTGGGAGGGACCAAAGGCGGCC |
| | | AGGATATAACTGACTTCACCATGCAATTTGTGTCTTCCTAA |
| Mouse IL-1β | 38 | MATVPELNCEMPPFDSDENDLFFEVDGPQKMKGCFQTFDLGCPDESIQLQISQ |
| amino acid | | QHINKSFRQAVSLIVAVEKLWQLPVSFPWTFQDEDMSTFFSFIFEEEPILCDSW |
| sequence | | DDDDNLLVCDVPIRQLHYRLRDEQQKSLVLSDPYELKALHLNGQNINQQVIFSM |
| | | SFVQGEPSNDKIPVALGLKGKNLYLSCVMKDGTPTLQLESVDPKQYPKKKMEK |
| | | RFVFNKIEVKSKVEFESAEFPNWYISTSQAEHKPVFLGNNSGQDIIDFTMESVSS |
| Mouse IL-1β | 39 | ATGGCAACTGTTCCTGAACTCAACTGTGAAATGCCACCTTTTGACAGTGATG |
| nucleic acid | | AGAATGACCTGTTCTTTGAAGTTGACGGACCCCAAAAGATGAAGGGCTGCT |
| sequence | | TCCAAACCTTTGACCTGGGCTGTCCTGATGAGAGCATCCAGCTTCAAATCTC |
| | | GCAGCAGCACATCAACAAGAGCTTCAGGCAGGCAGTATCACTCATTGTGGC |
| | | TGTGGAGAAGCTGTGGCAGCTACCTGTGTCTTTCCCGTGGACCTTCCAGGA |
| | | TGAGGACATGAGCACCTTCTTTTCCTTCATCTTTGAAGAAGAGCCCATCCTC |
| | | TGTGACTCATGGGATGATGATGATAACCTGCTGGTGTGTGACGTTCCCATTA |
| | | GACAACTGCACTACAGGCTCCGAGATGAACAACAAAAAAGCCTCGTGCTGT |
| | | CGGACCCATATGAGCTGAAAGCTCTCCACCTCAATGGACAGAATATCAACC |
| | | AACAAGTGATATTCTCCATGAGCTTTGTACAAGGAGAACCAAGCAACGACAA |
| | | AATACCTGTGGCCTTGGGCCTCAAAGGAAAGAATCTATACCTGTCCTGTGTA |
| | | ATGAAAGACGGCACACCCACCCTGCAGCTGGAGAGTGTGGATCCCAAGCA |
| | | ATACCCAAAGAAGAAGATGGAAAAACGGTTTGTCTTCAACAAGATAGAAGTC |
| | | AAGAGCAAAGTGGAGTTTGAGTCTGCAGAGTTCCCCAACTGGTACATCAGC |
| | | ACCTCACAAGCAGAGCACAAGCCTGTCTTCCTGGGAAACAACAGTGGTCAG |
| | | GACATAATTGACTTCACCATGGAATCCGTGTCTTCCTAA |
| Human IL-13 | 40 | MHPLLNPLLLALGLMALLLTTVIALTCLGGFASPGPVPPSTALRELIEELVNITQN |
| amino acid | | QKAPLCNGSMVWSINLTAGMYCAALESLINVSGCSAIEKTQRMLSGFCPHKVS |
| sequence | | AGQFSSLHVRDTKIEVAQFVKDLLLHLKKLFREGRFN |
| Human IL-13 | 41 | ATGCATCCGCTCCTCAATCCTCTCCTGTTGGCACTGGGCCTCATGGCGCTT |
| nucleic acid | | TTGTTGACCACGGTCATTGCTCTCACTTGCCTTGGCGGCTTTGCCTCCCCA |
| sequence | | GGCCCTGTGCCTCCCTCTACAGCCCTCAGGGAGCTCATTGAGGAGCTGGT |
| | | CAACATCACCCAGAACCAGAAGGCTCCGCTCTGCAATGGCAGCATGGTATG |
| | | GAGCATCAACCTGACAGCTGGCATGTACTGTGCAGCCCTGGAATCCCTGAT |
| | | CAACGTGTCAGGCTGCAGTGCCATCGAGAAGACCCAGAGGATGCTGAGCG |
| | | GATTCTGCCCGCACAAGGTCTCAGCTGGGCAGTTTTCCAGCTTGCATGTCC |
| | | GAGACACCAAAATCGAGGTGGCCCAGTTTGTAAAGGACCTGCTCTTACATTT |
| | | AAAGAAACTTTTTCGCGAGGGACGGTTCAACTGA |
| Mouse IL-13 | 42 | MALWVTAVLALACLGGLAAPGPVPRSVSLPLTLKELIEELSNITQDQTPLCNGS |
| amino acid | | MVWSVDLAAGGFCVALDSLTNISNCNAIYRTQRILHGLCNRKAPTTVSSLPDTKI |
| sequence | | EVAHFITKLLSYTKQLFRHGPF |
| Mouse IL-13 | 43 | ATGGCGCTCTGGGTGACTGCAGTCCTGGCTCTTGCTTGCCTTGGTGGTCTC |
| nucleic acid | | GCCGCCCCAGGGCCGGTGCCAAGATCTGTGTCTCTCCCTCTGACCCTTAAG |
| sequence | | GAGCTTATTGAGGAGCTGAGCAACATCACACAAGACCAGACTCCCCTGTGC |
| | | AACGGCAGCATGGTATGGAGTGTGGACCTGGCCGCTGGCGGGTTCTGTGT |
| | | AGCCCTGGATTCCCTGACCAACATCTCCAATTGCAATGCCATCTACAGGAC |
| | | CCAGAGGATATTGCATGGCCTCTGTAACCGCAAGGCCCCCACTACGGTCTC |
| | | CAGCCTCCCCGATACCAAAATCGAAGTAGCCCACTTTATAACAAAACTGCTC |
| | | AGCTACACAAAGCAACTGTTTCGCCACGGCCCCTTCTAA |
| Human TNF-α | 44 | MSTESMIRDVELAEEALPKKTGGPQGSRRCLFLSLFSFLIVAGATTLFCLLHFGV |
| amino acid | | IGPQREEFPRDLSLISPLAQAVRSSSRTPSDKPVAHVVANPQAEGQLQWLNRR |
| sequence | | ANALLANGVELRDNQLVVPSEGLYLIYSQVLFKGQGCPSTHVLLTHTISRIAVSY |
| | | QTKVNLLSAIKSPCQRETPEGAEAKPWYEPIYLGGVFQLEKGDRLSAEINRPDY |
| | | LDFAESGQVYFGIIAL |
| Human TNF-α | 45 | ATGAGCACTGAAAGCATGATCCGGGACGTGGAGCTGGCCGAGGAGGCGCT |
| nucleic acid | | CCCCAAGAAGACAGGGGGGCCCCAGGGCTCCAGGCGGTGCTTGTTCCTCA |
| sequence | | GCCTCTTCTCCTTCCTGATCGTGGCAGGCGCCACCACGCTCTTCTGCCTGC |
| | | TGCACTTTGGAGTGATCGGCCCCCAGAGGGAAGAGTTCCCCAGGGACCTC |
| | | TCTCTAATCAGCCCTCTGGCCCAGGCAGTCAGATCATCTTCTCGAACCCCG |
| | | AGTGACAAGCCTGTAGCCCATGTTGTAGCAAACCCTCAAGCTGAGGGGCAG |
| | | CTCCAGTGGCTGAACCGCCGGGCCAATGCCCTCCTGGCCAATGGCGTGGA |
| | | GCTGAGAGATAACCAGCTGGTGGTGCCATCAGAGGGCCTGTACCTCATCTA |
| | | CTCCCAGGTCCTCTTCAAGGGCCAAGGCTGCCCCTCCACCCATGTGCTCCT |
| | | CACCCACACCATCAGCCGCATCGCCGTCTCCTACCAGACCAAGGTCAACCT |
| | | CCTCTCTGCCATCAAGAGCCCCTGCCAGAGGGAGACCCCAGAGGGGGCTG |
| | | AGGCCAAGCCCTGGTATGAGCCCATCTATCTGGGAGGGGTCTTCCAGCTG |
| | | GAGAAGGGTGACCGACTCAGCGCTGAGATCAATCGGCCCGACTATCTCGA |
| | | CTTTGCCGAGTCTGGGCAGGTCTACTTTGGGATCATTGCCCTGTGA |
| Mouse TNF-α | 46 | MSTESMIRDVELAEEALPQKMGGFQNSRRCLCLSLFSFLLVAGATTLFCLLNFG |
| amino acid | | VIGPQRDEKFPNGLPLISSMAQTLTLRSSSQNSSDKPVAHVVANHQVEEQLEW |
| sequence | | LSQRANALLANGMDLKDNQLVVPADGLYLVYSQVLFKGQGCPDYVLLTHTVSR |
| | | FAISYQEKVNLLSAVKSPCPKDTPEGAELKPWYEPIYLGGVFQLEKGDQLSAEV |
| | | NLPKYLDFAESGQVYFGVIAL |
| Mouse TNF-α | 47 | ATGAGCACAGAAAGCATGATCCGCGACGTGGAACTGGCAGAAGAGGCACT |
| nucleic acid | | CCCCCAAAAGATGGGGGGCTTCCAGAACTCCAGGCGGTGCCTATGTCTCA |
| sequence | | GCCTCTTCTCATTCCTGCTTGTGGCAGGGGCCACCACGCTCTTCTGTCTAC |
| | | TGAACTTCGGGGTGATCGGTCCCCAAAGGGATGAGAAGTTCCCAAATGGCC |
| | | TCCCTCTCATCAGTTCTATGGCCCAGACCCTCACACTCAGATCATCTTCTCA |
| | | AAATTCGAGTGACAAGCCTGTAGCCCACGTCGTAGCAAACCACCAAGTGGA |
| | | GGAGCAGCTGGAGTGGCTGAGCCAGCGCGCCAACGCCCTCCTGGCCAAC |
| | | GGCATGGATCTCAAAGACAACCAACTAGTGGTGCCAGCCGATGGGTTGTAC |
| | | CTTGTCTACTCCCAGGTTCTCTTCAAGGGACAAGGCTGCCCCGACTACGTG |
| | | CTCCTCACCCACACCGTCAGCCGATTTGCTATCTCATACCAGGAGAAAGTC |
| | | AACCTCCTCTCTGCCGTCAAGAGCCCCTGCCCCAAGGACACCCCTGAGGG |
| | | GGCTGAGCTCAAACCCTGGTATGAGCCCATATACCTGGGAGGAGTCTTCCA |
| | | GCTGGAGAAGGGGGACCAACTCAGCGCTGAGGTCAATCTGCCCAAGTACT |
| | | TAGACTTTGCGGAGTCCGGGCAGGTCTACTTTGGAGTCATTGCTCTGTGA |

### Example 3: Examples of Embodiments

The examples set forth below illustrate certain embodiments and do not limit the technology.

A1. A method for assessing activity of an IL-40 polypeptide comprising:
a) contacting a cell with a first composition comprising an IL-40 polypeptide;
b) measuring production by the cell of one or more cytokines and/or chemokines chosen from CCL2, CCL3, CCL4, CCL5, CCL11, CXCL8, CXCL10, and IL-1RA, thereby measuring cytokine production; and
c) detecting the activity of the IL-40 polypeptide in the first composition according to the cytokine production measured in (b).

A2. The method of embodiment A1, wherein the production of the one or more cytokines and/or chemokines is increased compared to the production by a cell not contacted with the first composition.

A3. The method of embodiment A1 or A2, wherein (a) comprises contacting the cell with a second composition comprising a co-stimulant.

A4. The method of embodiment A3, wherein the co-stimulant comprises IFN-γ.

A5. The method of embodiment A3 or A4, wherein (a) comprises simultaneously contacting the cell with the first composition and the second composition.

A6. The method of embodiment A3 or A4, wherein (a) comprises contacting the cell with the second composition prior to contacting the cell with the first composition.

A7. The method of any one of embodiment A3 to A6, wherein the production of the one or more cytokines and/or chemokines is increased compared to the production by a cell not contacted with the first composition and the second composition.

A8. A method for assessing activity of an IL-40 polypeptide comprising:
a) contacting a cell with a first composition comprising an IL-40 polypeptide and a second composition comprising a co-stimulant;
b) measuring production by the cell of one or more cytokines and/or chemokines chosen from CCL2, CCL3, CCL4, CCL5, CCL11, CCL17, CCL20, CXCL1, CXCL5, CXCL8, CXCL9, CXCL10, CXCL11, IL-1RA, and IL-6, thereby measuring cytokine production; and
c) detecting the activity of the IL-40 polypeptide in the first composition according to the cytokine production measured in (b).

A9. The method of embodiment A8, wherein the production of the one or more cytokines and/or chemokines is increased compared to the production by a cell not contacted with the first composition and the second composition.

A10. The method of embodiment A8 or A9, wherein the co-stimulant comprises IFN-γ.

A11. The method of any one of embodiments A8 to A10, wherein (a) comprises simultaneously contacting the cell with the first composition and the second composition.

A12. The method of any one of embodiments A8 to A10, wherein (a) comprises contacting the cell with the second composition prior to contacting the cell with the first composition.

A13. The method of any one of embodiments A1 to A12, wherein the cell is from a subject.

A14. The method of any one of embodiments A1 to A12, wherein the cell is from a cell line.

A15. The method of any one of embodiments A1 to A14, wherein the cell is an isolated cell.

A16. The method of any one of embodiments A1 to A15, wherein the cell is an immune cell.

A17. The method of embodiment A16, wherein the cell is a monocyte.

A18. The method of embodiment A16, wherein the cell is a macrophage.

A19. The method of any one of embodiments A1 to A15, wherein the cell is a non-immune cell.

A20. The method of embodiment A19, wherein the cell is a stromal cell or a cell derived from the central nervous system.

A21. The method of any one of embodiments A1 to A20, wherein the method is performed ex vivo or in vitro.

A22. The method of any one of embodiments A1 to A21, wherein the IL-40 polypeptide is a recombinant IL-40 polypeptide.

A23. The method of any one of embodiments A1 to A21, wherein the IL-40 polypeptide is a human IL-40 polypeptide.

A24. The method of embodiment A23, wherein the IL-40 polypeptide is a recombinant human IL-40 polypeptide.

A25. The method of embodiment A23 or A24, wherein the IL-40 polypeptide comprises the amino acid sequence of SEQ ID NO: 1.

A26. The method of embodiment A23 or A24, wherein the IL-40 polypeptide comprises amino acids 21-265 of SEQ ID NO: 1.

A27. The method of embodiment A23 or A24, wherein the IL-40 polypeptide comprises a fragment of the amino acid sequence of SEQ ID NO: 1.

A28. The method of embodiment A23 or A24, wherein the IL-40 polypeptide comprises one or more amino acid substitutions in the amino acid sequence of SEQ ID NO: 1.

A29. The method of embodiment A23 or A24, wherein the IL-40 polypeptide comprises a fragment comprising one or more amino acid substitutions in the amino acid sequence of SEQ ID NO: 1.

A30. The method of any one of embodiments A1 to A21, wherein the IL-40 polypeptide is a mouse IL-40 polypeptide.

A31. The method of embodiment A30, wherein the IL-40 polypeptide is a recombinant mouse IL-40 polypeptide.

A32. The method of embodiment A30 or A31, wherein the IL-40 polypeptide comprises the amino acid sequence of SEQ ID NO: 3.

A33. The method of embodiment A30 or A31, wherein the IL-40 polypeptide comprises amino acids 19-252 of SEQ ID NO: 3.

A34. The method of embodiment A30 or A31, wherein the IL-40 polypeptide comprises a fragment of the amino acid sequence of SEQ ID NO: 3.

A35. The method of embodiment A30 or A31, wherein the IL-40 polypeptide comprises one or more amino acid substitutions in the amino acid sequence of SEQ ID NO: 3.

A36. The method of embodiment A30 or A31, wherein the IL-40 polypeptide comprises a fragment comprising one or more amino acid substitutions in the amino acid sequence of SEQ ID NO: 3.

A37. The method of any one of embodiments A1 to A36, wherein the IL-40 polypeptide comprises one or more chemical modifications.

A38. The method of any one of embodiments A1 to A37, wherein the IL-40 polypeptide comprises a tag.

A39. The method of any one of embodiments A1 to A38, wherein the IL-40 polypeptide comprises a detectable label.

A40. The method of any one of embodiments A1 to A39, wherein the IL-40 polypeptide comprises a fused polypeptide.

B1. A method for assessing activity of an IL-40 polypeptide comprising:
a) contacting a population of monocytes with a first composition comprising an IL-40 polypeptide;
b) detecting monocyte to macrophage differentiation in the population; and
c) assessing the activity of the IL-40 polypeptide in the first composition according to the monocyte to macrophage differentiation detected in (b).

B2. The method of embodiment 1, wherein the monocyte to macrophage differentiation is detected according to changes in cell morphology.

B3. The method of embodiment B2, wherein at least about 10% of the cells in the population show changes in cell morphology.

B4. The method of embodiment 1, wherein the monocyte to macrophage differentiation is detected according to expression of one or more markers chosen from CD14, Ly6C, CD115, CD15s, CD33, CD44, CD81, CD49e, CD18, CD11b, CD54 (ICAM-1), CD11c, CD68, CD80, CD86, CD163, IL-1R, and CD200R.

B4.1 The method of embodiment 1, wherein the monocyte to macrophage differentiation is detected according to increased expression of one or more markers chosen from CD44, CD81, CD49e, CD18, CD11b, CD54 (ICAM-1), CD11c, CD68, CD80, CD86, CD163, IL-1R, and CD200R.

B4.2 The method of embodiment 1, wherein the monocyte to macrophage differentiation is detected according to decreased expression of one or more markers chosen from CD14, Ly6C, CD115, CD15s, and CD33.

B5. The method of embodiment 1, wherein the monocyte to macrophage differentiation is detected according to increased production of one or more chemokines chosen from CCL10, CCL11, CCL5, CCL8, CCL9, CCL2, CCL3, CCL4, CCL17, CCL22, CCL24, CCL1, CCR2, CCL5, CXCL10, CXCL8, CXCL9, and CXCL16.

B5.1 The method of embodiment 1, wherein the monocyte to macrophage differentiation is detected according to increased production of one or more cytokines chosen from TNF, IL-1 beta, IL-6, IL-12, IL-23, IL-10, TGF-beta, IL-1RA, IL-1, TNF-alpha, and IL-10.

B6. The method of embodiment 1, wherein (a) comprises contacting the population of monocytes with a second composition comprising a co-stimulant.

B7. The method of embodiment B6, wherein the co-stimulant comprises IFN-γ.

B8. The method of embodiment B6 or B7, wherein (a) comprises simultaneously contacting the population of monocytes with the first composition and the second composition.

B9. The method of embodiment B6 or B7, wherein (a) comprises contacting the population of monocytes with the second composition prior to contacting the cell with the first composition.

B10. The method of any one of embodiments B6 to B9, wherein the monocyte to macrophage differentiation is detected according to changes in cell morphology.

B11. The method of embodiment B10, wherein at least about 30% of the cells in the population show changes in cell morphology.

B12. The method of any one of embodiments B6 to B9, wherein the monocyte to macrophage differentiation is detected according to expression of one or more markers chosen from CD14, Ly6C, CD115, CD15s, CD33, CD44, CD81, CD49e, CD18, CD11b, CD54 (ICAM-1), CD11c, CD68, CD80, CD86, CD163, IL-1R, and CD200R.

B12.1 The method of any one of embodiments B6 to B9, wherein the monocyte to macrophage differentiation is detected according to increased expression of one or more markers chosen from CD44, CD81, CD49e, CD18, CD11b, CD54 (ICAM-1), CD11c, CD68, CD80, CD86, CD163, IL-1R, and CD200R.

B12.2 The method of any one of embodiments B6 to B9, wherein the monocyte to macrophage differentiation is detected according to decreased expression of one or more markers chosen from CD14, Ly6C, CD115, CD15s, and CD33.

B13. The method of any one of embodiments B6 to B9, wherein the monocyte to macrophage differentiation is detected according to increased production of one or more chemokines chosen from CCL10, CCL11, CCL5, CCL8, CCL9, CCL2, CCL3, CCL4, CCL17, CCL22, CCL24, CCL1, CCR2, CCL5, CXCL10, CXCL8, CXCL9, and CXCL16.

B13.1 The method of any one of embodiments B6 to B9, wherein the monocyte to macrophage differentiation is detected according to increased production of one or more cytokines chosen from TNF, IL-1 beta, IL-6, IL-12, IL-23, IL-10, TGF-beta, IL-1RA, IL-1, TNF-alpha, and IL-10.

B14. The method of any one of embodiments 1 to B13.1, wherein the population of monocytes is from a subject.

B15. The method of any one of embodiments 1 to B13.1, wherein the population of monocytes is from a cell line.

B16. The method of any one of embodiments 1 to B15, wherein the population of monocytes comprises isolated monocytes.

B17. The method of any one of embodiments 1 to B16, wherein the method is performed ex vivo or in vitro.

B18. The method of any one of embodiments 1 to B17, wherein the IL-40 polypeptide is a recombinant IL-40 polypeptide.

B19. The method of any one of embodiments 1 to B17, wherein the IL-40 polypeptide is a human IL-40 polypeptide.

B20. The method of embodiment B19, wherein the IL-40 polypeptide is a recombinant human IL-40 polypeptide.

B21. The method of embodiment B19 or B20, wherein the IL-40 polypeptide comprises the amino acid sequence of SEQ ID NO: 1.

B22. The method of embodiment B19 or B20, wherein the IL-40 polypeptide comprises amino acids 21-265 of SEQ ID NO: 1.

B23. The method of embodiment B19 or B20, wherein the IL-40 polypeptide comprises a fragment of the amino acid sequence of SEQ ID NO: 1.

B24. The method of embodiment B19 or B20, wherein the IL-40 polypeptide comprises one or more amino acid substitutions in the amino acid sequence of SEQ ID NO: 1.

B25. The method of embodiment B19 or B20, wherein the IL-40 polypeptide comprises a fragment comprising one or more amino acid substitutions in the amino acid sequence of SEQ ID NO: 1.

B26. The method of any one of embodiments 1 to B17, wherein the IL-40 polypeptide is a mouse IL-40 polypeptide.

B27. The method of embodiment B26, wherein the IL-40 polypeptide is a recombinant mouse IL-40 polypeptide.

B28. The method of embodiment B26 or B27, wherein the IL-40 polypeptide comprises the amino acid sequence of SEQ ID NO: 3.

B29. The method of embodiment B26 or B27, wherein the IL-40 polypeptide comprises amino acids 19-252 of SEQ ID NO: 3.

B30. The method of embodiment B26 or B27, wherein the IL-40 polypeptide comprises a fragment of the amino acid sequence of SEQ ID NO: 3.

B31. The method of embodiment B26 or B27, wherein the IL-40 polypeptide comprises one or more amino acid substitutions in the amino acid sequence of SEQ ID NO: 3.

B32. The method of embodiment B26 or B27, wherein the IL-40 polypeptide comprises a fragment comprising one or more amino acid substitutions in the amino acid sequence of SEQ ID NO: 3.

B33. The method of any one of embodiments 1 to B32, wherein the IL-40 polypeptide comprises one or more chemical modifications.

B34. The method of any one of embodiments 1 to B33, wherein the IL-40 polypeptide comprises a tag.

B35. The method of any one of embodiments 1 to B34, wherein the IL-40 polypeptide comprises a detectable label.

B36. The method of any one of embodiments 1 to B35, wherein the IL-40 polypeptide comprises a fused polypeptide.

C1. A method for inducing differentiation of a monocyte to a macrophage, comprising contacting a monocyte with a first composition comprising an IL-40 polypeptide.

C2. The method of embodiment C1, wherein the differentiation of a monocyte to a macrophage is characterized by changes in cell morphology.

C3. The method of embodiment C2, wherein at least about 10% of cells in a population of monocytes show changes in cell morphology.

C4. The method of embodiment C1, wherein the differentiation of a monocyte to a macrophage is characterized by expression of one or more markers chosen from CD14, Ly6C, CD115, CD15s, CD33, CD44, CD81, CD49e, CD18, CD11b, CD54 (ICAM-1), CD11c, CD68, CD80, CD86, CD163, IL-1R, and CD200R.

C5. The method of embodiment C1, wherein the differentiation of a monocyte to a macrophage is characterized by increased expression of one or more markers chosen from CD44, CD81, CD49e, CD18, CD11b, CD54 (ICAM-1), CD11c, CD68, CD80, CD86, CD163, IL-1R, and CD200R.

C6. The method of embodiment C1, wherein the differentiation of a monocyte to a macrophage is characterized by decreased expression of one or more markers chosen from CD14, Ly6C, CD115, CD15s, and CD33.

C7. The method of embodiment C1, wherein the differentiation of a monocyte to a macrophage is characterized by increased production of one or more chemokines chosen from CCL10, CCL11, CCL5, CCL8, CCL9, CCL2, CCL3, CCL4, CCL17, CCL22, CCL24, CCL1, CCR2, CCL5, CXCL10, CXCL8, CXCL9, and CXCL16.

C8. The method of embodiment C1, wherein the differentiation of a monocyte to a macrophage is characterized by increased production of one or more cytokines chosen from TNF, IL-1 beta, IL-6, IL-12, IL-23, IL-10, TGF-beta, IL-1RA, IL-1, TNF-alpha, and IL-10.

C9. The method of embodiment C1, comprising contacting the monocyte with a second composition comprising a co-stimulant.

C10. The method of embodiment C9, wherein the co-stimulant comprises IFN-γ.

C11. The method of embodiment C9 or C10, comprising simultaneously contacting the monocyte with the first composition and the second composition.

C12. The method of embodiment C9 or C10, comprising contacting the monocyte with the second composition prior to contacting the monocyte with the first composition.

C13. The method of any one of embodiments C9 to C12, wherein the differentiation of a monocyte to a macrophage is characterized by changes in cell morphology.

C14. The method of embodiment C13, wherein at least about 30% of cells in a population of monocytes show changes in cell morphology.

C15. The method of any one of embodiments C9 to C12, wherein the differentiation of a monocyte to a macrophage is characterized by expression of one or more markers chosen from CD14, Ly6C, CD115, CD15s, CD33, CD44, CD81, CD49e, CD18, CD11b, CD54 (ICAM-1), CD11c, CD68, CD80, CD86, CD163, IL-1R, and CD200R.

C16. The method of any one of embodiments C9 to C12, wherein the differentiation of a monocyte to a macrophage is characterized by increased expression of one or more markers chosen from CD44, CD81, CD49e, CD18, CD11b, CD54 (ICAM-1), CD11c, CD68, CD80, CD86, CD163, IL-1R, and CD200R.

C17. The method of any one of embodiments C9 to C12, wherein the differentiation of a monocyte to a macrophage is characterized by decreased expression of one or more markers chosen from CD14, Ly6C, CD115, CD15s, and CD33.

C18. The method of any one of embodiments C9 to C12, wherein the differentiation of a monocyte to a macrophage is characterized by increased production of one or more chemokines chosen from CCL10, CCL11, CCL5, CCL8, CCL9, CCL2, CCL3, CCL4, CCL17, CCL22, CCL24, CCL1, CCR2, CCL5, CXCL10, CXCL8, CXCL9, and CXCL16.

C19. The method of any one of embodiments C9 to C12, wherein the differentiation of a monocyte to a macrophage is characterized by increased production of one or more cytokines chosen from TNF, IL-1 beta, IL-6, IL-12, IL-23, IL-10, TGF-beta, IL-1RA, IL-1, TNF-alpha, and IL-10.

C20. The method of any one of embodiments C1 to C19, wherein the monocyte is from a subject.

C21. The method of any one of embodiments C1 to C19, wherein the monocyte is from a cell line.

C22. The method of any one of embodiments C1 to C21, wherein the monocyte is an isolated monocyte.

C23. The method of any one of embodiments C1 to C22, wherein the method is performed ex vivo or in vitro.

C24. The method of any one of embodiments C1 to C23, wherein the IL-40 polypeptide is a recombinant IL-40 polypeptide.

C25. The method of any one of embodiments C1 to C23, wherein the IL-40 polypeptide is a human IL-40 polypeptide.

C26. The method of embodiment C25, wherein the IL-40 polypeptide is a recombinant human IL-40 polypeptide.

C27. The method of embodiment C25 or C26, wherein the IL-40 polypeptide comprises the amino acid sequence of SEQ ID NO: 1 or amino acids 21-265 of SEQ ID NO: 1; or a functional fragment thereof; or a modified polypeptide thereof; or a modified functional fragment thereof.

C28. The method of any one of embodiments C1 to C23, wherein the IL-40 polypeptide is a mouse IL-40 polypeptide.

C29. The method of embodiment C28, wherein the IL-40 polypeptide is a recombinant mouse IL-40 polypeptide.

C30. The method of embodiment C28 or C29, wherein the IL-40 polypeptide comprises the amino acid sequence of SEQ ID NO: 3 or amino acids 19-252 of SEQ ID NO: 3; or a functional fragment thereof; or a modified polypeptide thereof; or a modified functional fragment thereof.

D1. A kit, comprising:
a) a first composition comprising an IFN-γ polypeptide;
b) one or more components for measuring cytokine and/or chemokine production, wherein the cytokines and/or chemokines are chosen from one or more of CCL2, CCL3, CCL4, CCL5, CCL11, CCL17, CCL20, CXCL1, CXCL5, CXCL8, CXCL9, CXCL10, CXCL11, IL-1RA, and IL-6; and
c) instructions for use.

D2. The kit of embodiment D1, further comprising a second composition comprising an IL-40 polypeptide.

D3. The kit of embodiment D2, wherein the IL-40 polypeptide is a recombinant IL-40 polypeptide.

D4. The kit of embodiment D2, wherein the IL-40 polypeptide is a human IL-40 polypeptide.

D5. The kit of embodiment D4, wherein the IL-40 polypeptide is a recombinant human IL-40 polypeptide.

D6. The kit of embodiment D4 or D5, wherein the IL-40 polypeptide comprises the amino acid sequence of SEQ ID NO: 1.

D7. The kit of embodiment D4 or D5, wherein the IL-40 polypeptide comprises amino acids 21-265 of SEQ ID NO: 1.

D8. The kit of embodiment D4 or D5, wherein the IL-40 polypeptide comprises a fragment of the amino acid sequence of SEQ ID NO: 1.

D9. The kit of embodiment D4 or D5, wherein the IL-40 polypeptide comprises one or more amino acid substitutions in the amino acid sequence of SEQ ID NO: 1.

D10. The kit of embodiment D4 or D5, wherein the IL-40 polypeptide comprises a fragment comprising one or more amino acid substitutions in the amino acid sequence of SEQ ID NO: 1.

D11. The kit of embodiment D2, wherein the IL-40 polypeptide is a mouse IL-40 polypeptide.

D12. The kit of embodiment D10, wherein the IL-40 polypeptide is a recombinant mouse IL-40 polypeptide.

D13. The kit of embodiment D11 or D12, wherein the IL-40 polypeptide comprises the amino acid sequence of SEQ ID NO: 3.

D14. The kit of embodiment D11 or D12, wherein the IL-40 polypeptide comprises amino acids 19-252 of SEQ ID NO: 3.

D15. The kit of embodiment D11 or D12, wherein the IL-40 polypeptide comprises a fragment of the amino acid sequence of SEQ ID NO: 3.

D16. The kit of embodiment D11 or D12, wherein the IL-40 polypeptide comprises one or more amino acid substitutions in the amino acid sequence of SEQ ID NO: 3.

D17. The kit of embodiment D11 or D12, wherein the IL-40 polypeptide comprises a fragment comprising one or more amino acid substitutions in the amino acid sequence of SEQ ID NO: 3.

D18. The kit of any one of embodiments D2 to D17, wherein the IL-40 polypeptide comprises one or more chemical modifications.

D19. The kit of any one of embodiments D2 to D18, wherein the IL-40 polypeptide comprises a tag.

D20. The kit of any one of embodiments D2 to D19, wherein the IL-40 polypeptide comprises a detectable label.

D21. The kit of any one of embodiments D2 to D20, wherein the IL-40 polypeptide comprises a fused polypeptide.

D22. The kit of any one of embodiments D1 to D21, wherein each of the one or more components for measuring cytokine and/or chemokine production comprises a binding molecule that immunospecifically binds to one of the cytokines and/or chemokines under binding conditions.

D23. The kit of any one of embodiments D1 to D22, further comprising a cell or a population of cells.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1.-144. (canceled)

145. A kit, comprising:
a) a first composition comprising one more polypeptides chosen from Interferon-gamma (IFN-γ), granulocyte-macrophage colony-stimulating factor (GM-CSF), interferon-alpha (IFN-α), interleukin 1 beta (IL-1), interleukin 4 (IL-4), interleukin 10 (IL-10), interleukin 13 (IL-13), macrophage colony-stimulating factor (M-CSF), transforming growth factor beta (TGF-0), and tumor necrosis factor alpha (TNF-α);
b) a second composition comprising an Interleukin 40 (IL-40) polypeptide;
b) one or more components for measuring cytokine, chemokine, and/or growth factor production, wherein the cytokines, chemokines, and/or growth factors are chosen from one or more of chemokine (C-C motif) ligand 2 (CCL2), chemokine (C-C motif) ligand 3 (CCL3), chemokine (C-C motif) ligand 4 (CCL4), chemokine (C-C motif) ligand 5 (CCL5), chemokine (C-C motif) ligand 11 (CCL11), chemokine (C-C motif) ligand 17 (CCL17), chemokine (C-C motif) ligand 20 (CCL20), chemokine (C-X-C motif) ligand 1 (CXCL1), chemokine (C-X-C motif) ligand 5 (CXCL5), chemokine (C-X-C motif) ligand 8 (CXCL8), chemokine (C-X-C motif) ligand 9 (CXCL9), chemokine (C-X-C motif) ligand 10 (CXCL10), chemokine (C-X-C motif) ligand 11 (CXCL11), interleukin-1 receptor antagonist (IL-1RA), IL-6, erythropoietin, platelet derived growth factor AA (PDGF-AA), and vascular endothelial growth factor (VEGF); and
c) instructions for use.

146. A method for characterizing IL-40 expression on a cell, comprising:
a) contacting the cell with a first composition comprising a stimulant;
b) contacting the cell with a second composition comprising an IL-40 polypeptide and a fusion tag or detectable label under conditions to bind said second composition to an IL-40 receptor on the cell, wherein the binding generates the production of a receptor/IL-40 polypeptide complex;
c) detecting the receptor/IL-40 polypeptide complex; wherein the complex comprises cytokines, chemokines, and/or growth factors and wherein the one or more cytokines, chemokines, and/or growth factors is selected from the group consisting of chemokine (C-C motif) ligand 2 (CCL2), chemokine (C-C motif) ligand 3 (CCL3), chemokine (C-C motif) ligand 4 (CCL4), chemokine (C-C motif) ligand 5 (CCL5), chemokine (C-C motif) ligand 11 (CCL11), chemokine (C-X-C motif) ligand 8 (CXCL8), chemokine (C-X-C motif) ligand 10 (CXCL10), interleukin-1 receptor antagonist (IL-1RA), erythropoietin, platelet derived growth factor AA (PDGF-AA) and vascular endothelial growth factor (VEGF); and
d) characterizing the cell based on the detection of the receptor/IL-40 polypeptide complex the cell.

147. The method of claim 146, wherein the detecting comprises the presence of the receptor/IL-40 polypeptide complex cell.

148. The method of claim 147, wherein the cell is characterized as expressing or capable of expressing a IL-40 receptor.

149. The method of claim 146, wherein the detecting comprises the absence of the receptor/meteorin-β polypeptide complex cell.

150. The method of claim 147, wherein the cell is characterized as not expressing or not capable of expressing a IL-40 receptor.

151. The method of claim 146, wherein the stimulant comprises a cytokine or a chemokine.

152. The method of claim 151, wherein the cytokine or chemokine comprises one or more polypeptides selected from IFN-γ, GM-CSF, IFN-α, IL-1β, IL-4, IL-10, IL-13, TNF-α, TGF-β, and M-CSF.

153. The method of claim 146, wherein the IL-40 polypeptide comprises a detectable label.

154. The method of claim 146, wherein the detecting comprises contacting the IL-40 polypeptide with an agent capable of detecting the detectable label.

155. The method of claim 154, wherein the agent is an antibody.

156. The method of claim 146, wherein the IL-40 polypeptide is a recombinant human IL-40 polypeptide or a recombinant mouse IL-40 polypeptide.

157. The method of claim 146, wherein the subject has or is suspected of having, a disease, disorder, syndrome, condition, infection or illness.

158. The method of claim 157, wherein the disease, disorder, syndrome, condition, infection or illness is characterized by increased expression of IL-40 and/or a IL-40 receptor.

159. The method of claim 157, wherein the disease, disorder, syndrome, condition, infection or illness is characterized by decreased expression of IL-40 and/or a IL-40 receptor.

160. A kit for assessing activity of an IL-40 polypeptide in a sample, comprising:
a) a population of monocytes;
b) a first comprising an IL-40 polypeptide;
c) an analysis device comprising a surface;
d) means for measuring monocyte to macrophage differentiation in the population; and
e) instructions for use.

161. The kit of claim 160, further comprising a second composition comprising a co-stimulant.

162. The kit of claim 161, wherein the co-stimulant comprises one or more polypeptides is selected from the group consisting of GM-CSF, IFN-α, IFN-γ, IL-10, IL-4, IL-10, IL-13, TNF-α, TGF-β, and M-CSF.

163. The kit of claim 160, wherein the means of d) comprises (i) a light source; and (ii) means for identifying the number of cells adhered to the surface of the analysis device.

164. The kit of claim 160, wherein the IL-40 polypeptide is a human IL-40 polypeptide or a mouse IL-40 polypeptide.


SEQUENCE LISTING (56 sequences)

SEQ ID NO: 1 - protein, 265 aa, Homo sapiens
MGLPGLFCLAVLAASSFSKAREEEITPVVSIAYKVLEVFPKGRWVLITCCAPQPPPPITYSLCGTKNIKVAKKVVKTHEPASFNLNVTLKSSPDLLTYFCWASSTSGAHVDSARLQMHWELWSKPVSELRANFTLQDRGAGPRVEMICQASSGSPPITNSLIGKDGQVHLQQRPCHRQPANFSFLPSQTSDWFWCQAANNANVQHSALTVVPPGGDQKMEDWQGPLESPILALPLYRSTRRLSEEEFGGFRIGNGEVRGRKAAAM

SEQ ID NO: 2 - DNA, 1090 bp, Homo sapiens
  source 1..1090: mol_type = genomic DNA
gccaggaactaggaggttctcactgcccgagcagaggccctacacccaccgaggcatggggctccctgggctgttctgcttggccgtgctggctgccagcagcttctccaaggcacgggaggaagaaattacccctgtggtctccattgcctacaaagtcctggaagttttccccaaaggccgctgggtgctcataacctgctgtgcaccccagccaccaccgcccatcacctattccctctgtggaaccaagaacatcaaggtggccaagaaggtggtgaagacccacgagccggcctccttcaacctcaacgtcacactcaagtccagtccagacctgctcacctacttctgctgggcgtcctccacctcaggtgcccatgtggacagtgccaggctacagatgcactgggagctgtggtccaagccagtgtctgagctgcgggccaacttcactctgcaggacagaggggcaggccccagggtggagatgatctgccaggcgtcctcgggcagcccacctatcaccaacagcctgatcgggaaggatgggcaggtccacctgcagcagagaccatgccacaggcagcctgccaacttctccttcctgccgagccagacatcggactggttctggtgccaggctgcaaacaacgccaatgtccagcacagcgccctcacagtggtgcccccaggtggtgaccagaagatggaggactggcagggtcccctggagagccccatccttgccttgccgctctacaggagcacccgccgtctgagtgaagaggagtttggggggttcaggatagggaatggggaggtcagaggacgcaaagcagcagccatgtagaatgaaccgtccagagagccaagcacggcagaggactgcaggccatcagcgtgcactgttcgtatttggagttcatgcaaaatgagtgtgttttagctgctcttgccacaaaaaaaaaaaaaaaaaaaaaagggtaactatgagagatggtggatatgttaacttgcttcgctataggaacctttgtgctatctatattatctatatgaatcccatcatatcaggttgtctacctta

SEQ ID NO: 3 - protein, 252 aa, Mus musculus
MALLQLLLFAMLAACGFSEEQTEGITIAYKVLEVYPQSRRVLITCDAPEASQPITYSLLASRGILVAKKVVHDSVPASFNINITIKSSPDLLTYSCQATSNSGTYGPSSRLQMYQELWAKPVSQLQADFVLRHGDSGPTVELSCLASSGSPPITYRLVGNGGRVLAQQRPLHGKPANFSLPLSQTTGWFQCEAENDVGVDSSARIPLPRAEARAKLVTTLAGELPLTPTCILAGSLVSIAVIASRMLSSTGL

SEQ ID NO: 4 - DNA, 1078 bp, Mus musculus
  source 1..1078: mol_type = genomic DNA
tagctgcaggcagccacccgaggagcatccaggggccttggggtggagaggccagacaggaagctttggacagcctgagccatacacagccccaacctcacagacgcatggcgctccttcagctgctcctctttgccatgctggctgcctgtggcttctcagaggagcagacagaaggcatcaccattgcctacaaagtactggaagtttatccccaaagccggagggtgcttataacctgcgatgcccctgaggcgtcccagcccatcacatactctctcctggctagccgaggtatcctggtggcaaaaaaggttgtgcatgactccgtgccggcctccttcaacatcaatatcaccatcaagtccagcccagacctgctcacctactcctgccaggcaacctcgaactctggcacctatggacccagcagcaggctccagatgtaccaggaactgtgggctaagccagtgtctcagctgcaggctgacttcgtcctacgccatggggactcgggccccactgtggagctgtcctgcctggcatcctcaggcagcccccccatcacctaccgcttggtggggaatggtgggcgtgttcttgcacagcaaaggccacttcatggaaaaccagccaacttctccctcccgctgtcccagaccactggttggttccagtgcgaagctgaaaacgatgtcggtgtggacagcagtgcccgcatcccgctgccccgagcagaggcccgagccaagctggtgaccaccctcgcaggggagctgcccctgacacccacctgtattctggctggcagcctcgtctccatagccgttattgcttccaggatgctgagctcgaccgggttgtgaccggaagacagagccatgggcttgcctccctgccctgtacaagaacccaccaatggagcaaaagagagcctgaggtgtgggggtagaaaaggggggttcagggctggagagatggctcagttacagcactgactgttcttccagaggtcccgagttcaattcccataatgtacttctacacataaaataaataattctttttttgttttgtt

SEQ ID NO: 5 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 6 - DNA, 1211 bp, Homo sapiens
  source 1..1211: mol_type = genomic DNA
acattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacta

SEQ ID NO: 7 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 8 - DNA, 1210 bp, Mus musculus
  source 1..1210: mol_type = genomic DNA
tatagctgccatcggctgacctagagaagacacatcagctgatcctttggaccctctgacttgagacagaagttctgggcttctcctcctgcggcctagctctgagacaatgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctgattcggggtggggaagagattgtcccaataagaataattctgccagcactatttgaatttttaaatctaaacctatttattaatatttaaaactatttatatggagaatctattttagatgcatcaaccaaagaagtatttatagtaacaacttatatgtgataagagtgaattcctattaatatatgtgttatttataatttctgtctcctcaactatttctctttgaccaattaattattctttctgactaattagccaagactgtgattgcggggttgtatctgggggtgggggacagccaagcggctgactgaactcagattgtagcttgtacctttacttcactgaccaataagaaacattcagagctgcagtgaccccgggaggtgctgctgatgggaggagatgtctacactccgggccagcgctttaacagcaggccagacagcactcgaatgtgtcaggtagtaacaggctgtccctgaaagaaagcagtgtctcaagagacttgacacctggtgcttccctatacagctgaaaactgtgactacacccgaatgacaaataactcgctcatttatagtttatcactgtctaattgcatatgaataaagtatacctttgcaaccaa

SEQ ID NO: 9 - protein, 486 aa, synthetic construct
  REGION 1..486: note = source = /note="Description of Artificial Sequence: Synthetic polypeptide"
LAREEEITPVVSIAYKVLEVFPKGRWVLITCCAPQPPPPITYSLCGTKNIKVAKKVVKTHEPASFNLNVTLKSSPDLLTYFCRASSTSGAHVDSARLQMHWELWSKPVSELRANFTLQDRGAGPRVEMICQASSGSPPITNSLIGKDGQVHLQQRPCHRQPANFSFLPSQTSDWFWCQAANNANVQHSALTVVPPGGDQKMEDWQGPLESPILALPLYRSTRRLSEEEFGGFRIGNGEVRGRKAAAMSRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1512 bp, synthetic construct
  misc_feature 1..1512: note = source = /note="Description of Artificial Sequence: Synthetic polynucleotide"
  source 1..1512: mol_type = other DNA
atgcccctgctgctgctgctgcccctgctgtgggccggcgccctggccctggctcgggaggaagaaattacccctgtggtctccattgcctacaaagtcctggaagttttccccaaaggccgctgggtgctcataacctgctgtgcaccccagccaccaccgcccatcacctattccctctgtggaaccaagaacatcaaggtggccaagaaggtggtgaagacccacgagccggcctccttcaacctcaacgtcacactcaagtccagtccagacctgctcacctacttctgccgggcgtcctccacctcaggtgcccatgtggacagtgccaggctacagatgcactgggagctgtggtccaagccagtgtctgagctgcgggccaacttcactctgcaggacagaggggcaggccccagggtggagatgatctgccaggcgtcctcgggcagcccacctatcaccaacagcctgatcgggaaggatgggcaggtccacctgcagcagagaccatgccacaggcagcctgccaacttctccttcctgccgagccagacatcggactggttctggtgccaggctgcaaacaacgccaatgtccagcacagcgccctcacagtggtgcccccaggtggtgaccagaagatggaggactggcagggtcccctggagagccccatccttgccttgccgctctacaggagcacccgccgtctgagtgaagaggagtttggggggttcaggatagggaatggggaggtcagaggacgcaaagcagcagccatgtctagaatcgagggccggatggaccccaagtcctgcgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaataataa

SEQ ID NO: 11 - protein, 330 aa, synthetic construct [sequence omitted]

SEQ ID NO: 12 - protein, 153 aa, Homo sapiens [sequence omitted]

SEQ ID NO: 13 - DNA, 462 bp, Homo sapiens
  source 1..462: mol_type = genomic DNA
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 14 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 15 - DNA, 423 bp, Mus musculus
  source 1..423: mol_type = genomic DNA
atgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtag

SEQ ID NO: 16 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - DNA, 537 bp, Homo sapiens
  source 1..537: mol_type = genomic DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 18 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 19 - DNA, 537 bp, Mus musculus
  source 1..537: mol_type = genomic DNA
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 20 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 21 - DNA, 1173 bp, Homo sapiens
  source 1..1173: mol_type = genomic DNA
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 22 - protein, 390 aa, Mus musculus
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 23 - DNA, 1173 bp, Mus musculus
  source 1..1173: mol_type = genomic DNA
atgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 24 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 25 - DNA, 788 bp, Homo sapiens
  source 1..788: mol_type = genomic DNA
agtacacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttg

SEQ ID NO: 26 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 27 - DNA, 426 bp, Mus musculus
  source 1..426: mol_type = genomic DNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatga

SEQ ID NO: 28 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 29 - DNA, 1665 bp, Homo sapiens
  source 1..1665: mol_type = genomic DNA
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatccttcagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtgtag

SEQ ID NO: 30 - protein, 552 aa, Mus musculus
MTARGAAGRCPSSTWLGSRLLLVCLLMSRSIAKEVSEHCSHMIGNGHLKVLQQLIDSQMETSCQIAFEFVDQEQLDDPVCYLKKAFFLVQDIIDETMRFKDNTPNANATERLQELSNNLNSCFTKDYEEQNKACVRTFHETPLQLLEKIKNFFNETKNLLEKDWNIFTKNCNNSFAKCSSRDVVTKPDCNCLYPKATPSSDPASASPHQPPAPSMAPLAGLAWDDSQRTEGSSLLPSELPLRIEDPGSAKQRPPRSTCQTLESTEQPNHGDRLTEDSQPHPSAGGPVPGVEDILESSLGTNWVLEEASGEASEGFLTQEAKFSPSTPVGGSIQAETDRPRALSASPFPKSTEDQKPVDITDRPLTEVNPMRPIGQTQNNTPEKTDGTSTLREDHQEPGSPHIATPNPQRVSNSATPVAQLLLPKSHSWGIVLPLGELEGKRSTRDRRSPAELEGGSASEGAARPVARFNSIPLTDTGHVEQHEGSSDPQIPESVFHLLVPGIILVLLTVGGLLFYKWKWRSHRDPQTLDSSVGRPEDSSLTQDEDRQVELPV

SEQ ID NO: 31 - DNA, 1659 bp, Mus musculus
  source 1..1659: mol_type = genomic DNA
atgaccgcgcggggcgccgcggggcgctgcccttcttcgacatggctgggctcccggctgctgctggtctgtctcctcatgagcaggagtattgccaaggaggtgtcagaacactgtagccacatgattgggaatggacacctgaaggtcctgcagcagttgatcgacagtcaaatggagacttcatgccagattgcctttgaatttgtagaccaggaacagctggatgatcctgtttgctacctaaagaaggccttttttctggtacaagacataatagatgagaccatgcgctttaaagacaacacccccaatgctaacgccaccgagaggctccaggaactctccaataacctgaacagctgcttcaccaaggactatgaggagcagaacaaggcctgtgtccgaactttccatgagactcctctccagctgctggagaagatcaagaacttctttaatgaaacaaagaatctccttgaaaaggactggaacatttttaccaagaactgcaacaacagctttgctaagtgctctagccgagatgtggtgaccaagcctgattgcaactgcctgtaccctaaagccacccctagcagtgacccggcctctgcctcccctcaccagccccccgccccctccatggcccctctggctggcttggcttgggatgattctcagaggacagagggcagctccctcttgcccagtgagcttccccttcgcatagaggacccaggcagtgccaagcagcgaccacccaggagtacctgccagaccctcgagtcaacagagcaaccaaaccatggggacagactcactgaggactcacaacctcatccttctgcgggggggcccgtccctggggtggaagacattcttgaatcttcactgggcactaactgggtcctagaagaagcttctggagaggctagtgagggatttttgacccaggaagcaaagttttccccctccacgcctgtagggggcagcatccaggcagagactgacagacccagggccctctcagcatctccattccctaaatcaacagaggaccaaaagccagtggatataacagacaggccgttgacagaggtgaaccctatgagacccattggccagacacagaataatactcctgagaagactgatggtacatccacgctgcgtgaagaccaccaggagccaggctctccccatattgcgacaccgaatccccaacgagtcagcaactcagccacccccgttgctcagttactgcttcccaaaagccactcttggggcattgtgctgccccttggggagcttgagggcaagagaagtaccagggatcgaaggagccccgcagagctggaaggaggatcagcaagtgagggggcagccaggcctgtggcccgttttaattccattcctttgactgacacaggccatgtggagcagcatgagggatcctctgacccccagatccctgagtctgtcttccacctgctggtgccgggcatcatcctagtcttgctgactgttgggggcctcctgttctacaagtggaagtggaggagccatcgagaccctcagacattggattcttctgtggggcgaccagaggacagctccctgacccaggatgaggacagacaggtggaactgccagtatag

SEQ ID NO: 32 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 33 - DNA, 567 bp, Homo sapiens
  source 1..567: mol_type = genomic DNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 34 - protein, 190 aa, Mus musculus
MARLCAFLMTLLVMSYWSTCSLGCDLPQTHNLRNKRALTLLVQMRRLSPLSCLKDRKDFRFPQEKVDAQQIQNAQAIPVLQELTQQVLNIFTSKDSSAAWDASLLDSFCNDLHQQLNDLKACVMQEVGVQEPPLTQEDYLLAVRTYFHRITVYLREKKHSPCAWEVVRAEVWRAMSSSAKLLARLSEEKE

SEQ ID NO: 35 - DNA, 573 bp, Mus musculus
  source 1..573: mol_type = genomic DNA
atggctaggctctgtgctttcctgatgaccctgctagtgatgagctactggtcaacctgctctctaggatgtgacctgcctcagactcataacctcaggaacaagagagccttgaccctcctggtacaaatgaggagactctcccctctctcctgcctgaaggacagaaaggactttagattcccccaggagaaggtggatgcccagcagatccagaatgctcaagccatccctgtcctacaagagctgacccagcaggtcctgaacatcttcacatcaaaggactcatctgctgcttgggatgcatccctcctagactcattctgcaatgacctccatcagcagctcaatgacctcaaagcctgtgtgatgcaggaggtgggggtgcaggaacctcccctgacccaggaagactacctgctggctgtgaggacatacttccacaggatcactgtgtacctgagagagaagaaacacagcccctgtgcctgggaggtggtcagagcagaagtctggagagccatgtcttcctcagccaagttgctggcaagactgagtgaggagaaggagtga

SEQ ID NO: 36 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 37 - DNA, 810 bp, Homo sapiens
  source 1..810: mol_type = genomic DNA
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 38 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 39 - DNA, 810 bp, Mus musculus
  source 1..810: mol_type = genomic DNA
atggcaactgttcctgaactcaactgtgaaatgccaccttttgacagtgatgagaatgacctgttctttgaagttgacggaccccaaaagatgaagggctgcttccaaacctttgacctgggctgtcctgatgagagcatccagcttcaaatctcgcagcagcacatcaacaagagcttcaggcaggcagtatcactcattgtggctgtggagaagctgtggcagctacctgtgtctttcccgtggaccttccaggatgaggacatgagcaccttcttttccttcatctttgaagaagagcccatcctctgtgactcatgggatgatgatgataacctgctggtgtgtgacgttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaa

SEQ ID NO: 40 - protein, 146 aa, Homo sapiens [sequence omitted]

SEQ ID NO: 41 - DNA, 441 bp, Homo sapiens
  source 1..441: mol_type = genomic DNA
atgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaactga

SEQ ID NO: 42 - protein, 131 aa, Mus musculus
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 43 - DNA, 396 bp, Mus musculus
  source 1..396: mol_type = genomic DNA
atggcgctctgggtgactgcagtcctggctcttgcttgccttggtggtctcgccgccccagggccggtgccaagatctgtgtctctccctctgacccttaaggagcttattgaggagctgagcaacatcacacaagaccagactcccctgtgcaacggcagcatggtatggagtgtggacctggccgctggcgggttctgtgtagccctggattccctgaccaacatctccaattgcaatgccatctacaggacccagaggatattgcatggcctctgtaaccgcaaggcccccactacggtctccagcctccccgataccaaaatcgaagtagcccactttataacaaaactgctcagctacacaaagcaactgtttcgccacggccccttctaa

SEQ ID NO: 44 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 45 - DNA, 702 bp, Homo sapiens
  source 1..702: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 46 - protein, 235 aa, Mus musculus
MSTESMIRDVELAEEALPQKMGGFQNSRRCLCLSLFSFLLVAGATTLFCLLNFGVIGPQRDEKFPNGLPLISSMAQTLTLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 47 - DNA, 708 bp, Mus musculus
  source 1..708: mol_type = genomic DNA
atgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtga

SEQ ID NO: 48 - protein, 9 aa, synthetic construct
  REGION 1..9: note = source = /note="Description of Artificial Sequence: Synthetic peptide"
DYKDDDDKG

SEQ ID NO: 49 - protein, 14 aa, synthetic construct
  REGION 1..14: note = source = /note="Description of Artificial Sequence: Synthetic peptide"
GKPIPNPLLGLDST

SEQ ID NO: 50 - protein, 10 aa, synthetic construct
  REGION 1..10: note = source = /note="Description of Artificial Sequence: Synthetic peptide"
EQKLISEEDL

SEQ ID NO: 51 - protein, 11 aa, synthetic construct
  REGION 1..11: note = source = /note="Description of Artificial Sequence: Synthetic peptide"
QPELAPEDPED

SEQ ID NO: 52 - protein, 9 aa, synthetic construct
  REGION 1..9: note = source = /note="Description of Artificial Sequence: Synthetic peptide"
YPYDVPDYA

SEQ ID NO: 53 - protein, 11 aa, synthetic construct
  REGION 1..11: note = source = /note="Description of Artificial Sequence: Synthetic peptide"
YTDIEMNRLGK

SEQ ID NO: 54 - protein, 6 aa, synthetic construct
  REGION 1..6: note = source = /note="Description of Artificial Sequence: Synthetic 6xHis tag"
HHHHHH

SEQ ID NO: 55 - protein, 5 aa, synthetic construct
  REGION 1..5: note = source = /note="Description of Artificial Sequence: Synthetic peptide"
  VARIANT 3: note = X residue can be repeated up to two times
  VARIANT 3: note = X can be any amino acid
CCXCC

SEQ ID NO: 56 - protein, 6 aa, synthetic construct
  REGION 1..6: note = source = /note="Description of Artificial Sequence: Synthetic peptide"
CCPGCC